(19) United States
(12) Patent Application Publication
(10) Pub. No.: US 2012/0095188 A1
(43) Pub. Date: Apr. 19, 2012

(54) ESTABLISHMENT OF INDUCED PLURIPOTENT STEM CELL USING CELL-PERMEABLE REPROGRAMMING TRANSCRIPTION FACTOR FOR CUSTOMIZED STEM CELL THERAPY

(75) Inventors: Daewoong Jo, Brentwood, TN (US); Jung-Hee Lim, Seoul (KR); Jungeun Kim, Seoul (KR); Sooyoung Jeong, Gyeonggi-do (KR); Inhee Jung, Gyeonggi-do (KR)

(73) Assignee: PROCELL THERAPEUTICS INC., Seoul (KR)

(21) Appl. No.: 13/263,955

(22) PCT Filed: Mar. 12, 2010
(86) PCT No.: PCT/KR2010/001559

§ 371 Date: Oct. 17, 2011

Related U.S. Application Data
(60) Provisional application No. 61/159,461, filed on Mar. 12, 2009.

(51) Int. Cl. C07K 19/00; C12P 21/02; C12N 1/21; C12N 15/62; C12N 15/70

(52) U.S. Cl. 530/350; 536/23.4; 435/320.1; 435/252.33; 435/69.7

(57) ABSTRACT

The present invention relates to a reprogramming transcription factor recombinant protein in which a macromolecule transduction domain (MTD) is fused to a reprogramming transcription factor to obtain cell permeability. The present invention also relates to a polynucleotide for coding said reprogramming transcription factor recombinant protein and to an expression vector of said cell-permeable reprogramming transcription factor recombinant protein. Treating a somatic cell with the cell-permeable reprogramming transcription factor recombinant protein induces the reprogramming of the stem cell-specific gene of the somatic cell, and thus can be effectively used in the establishment of an induced pluripotent stem cell (iPS cell) having characteristics similar to those of an embryonic stem cell in terms of morphology and genetics.

### FIELD OF THE INVENTION

The present invention relates to a reprogramming transcription factor recombinant protein in which a macromolecule transduction domain (MTD) is fused to a reprogramming transcription factor to obtain cell permeability. The present invention also relates to a polynucleotide which encodes said cell-permeable reprogramming transcription factor recombinant protein and an expression vector for said cell-permeable reprogramming transcription factor recombinant protein. Said cell-permeable reprogramming transcription factor recombinant protein can effectively increase the ability of reprogramming in a somatic cell, and thus can be useful in the establishment of an induced pluripotent stem cell (iPS cell).

### BACKGROUND

Embryonic stem (ES) cells are “pluripotent stem cells” derived from the inner cell mass (ICM) of blastocyst stage embryos. They can differentiate into three primary germ layers, i.e., endoderm, mesoderm, and ectoderm, which develop into the human body. The endoderm gives rise to cells of the thyroid gland, cells of the lung, and pancreatic cells; the mesoderm develops into cardiac muscle cells, muscle cells, kidney cells, and blood cells; and the ectoderm gives rise to not only skin cells, nervous cells, and pigment cells but also reproductive cells, i.e., ovum and sperm. Since embryonic stem cells can differentiate into such various types of cells, they are expected to play an important role as a tool for treating incurable diseases.

Embryonic stem cells can be established by using cells from frozen human embryos or by a somatic cell nuclear transfer technique in which the nucleus of a somatic cell is transferred to a denucleated egg cell. However, these two methods have ethical issues in that cells derived from human embryos are used and a large number of human eggs are required. In addition, low efficiency of the nuclear transfer and potential risk of fatal genetic defects caused by cloning are also problematic.

Another method of establishing embryonic stem cells that avoids the use of human embryos and eggs is to reprogram human somatic cells with reprogramming transcription factors. In this regard, it was reported that 24 candidate genes which were involved in the maintenance of embryonic stem cells were selected, and among them, four genes (Oct4, Sox2, c-Myc, and Klf4) were found to induce reprogramming of cells when transferred into the cell via viral vectors (Yamanaka, Cell 126: 633-678, 2006). It was shown that these four retrovirally-delivered factors were expressed in mouse fibroblast cells and induced reprogramming of the somatic cells to generate induced pluripotent stem cells. In addition, another study showed that human somatic cells were successfully reprogrammed by delivering Oct4 and Sox2 from the four reprogramming transcription factors in combination with new factors, i.e., Lin28 and Nanog (Yu J, Science 318(5858): 1917-20, 2007). Thus, it can be appreciated that among the six transcription factors experimented above (Oct4, Sox2, c-Myc, Klf4, Nanog, and Lin28), Oct4 and Sox2 are indispensible for induction of reprogramming, while c-Myc, Klf4, Nanog, and Lin28 can be optionally used with the two primary transcription factors in various combinations and play an important role in the establishment of pluripotent stem cells and the reprogramming process. In addition, the aforementioned genes are essential for pluripotency and self-renewal, and the maintenance of undifferentiation in embryonic stem cells. Transcription factors expressed from these genes function to activate one another and induce differentiation while suppressing inhibitory genes for self-renewal.

Oct-4, also known as POU domain class V, is an octamer transcription factor belonging to the POU family. This protein is known to be expressed specifically in undifferentiated cells such as embryonic stem cells and embryonic tumor cells. Further, it plays a critical role in the maintenance of pluripotency of the stem cells. If the level of Oct-4 expression is within a normal range, the self-renewal and pluripotency of stem cells are maintained. However, an expression level of Oct-4 higher than the normal range causes the stem cells to differentiate into primitive endoderm and primitive mesoderm. On the contrary, a low expression level of Oct-4 leads the stem cells to develop into trophectoderm. Thus, Oct-4 is vital for regulating pluripotency of embryonic stem cells and cell differentiation. Oct-4 was also reported to interact with Sox2 to synergically increase expression of genes in the lower stream, i.e., Fgf4, Utf-1, Nanog, and Sox2.

The genes of the Sox family relate to unipotent stem cells as well as multipotent stem cells. Among them, Sox2 (SRY-type high mobility group box 2) transcription factor is a member of the Sox family comprising an HMG box DNA binding motif. Sox 2 is the only one, among 20 proteins of the Sox family, that plays an important role in maintaining the pluripotency of embryonic stem cells. Like Oct-4, if the expression of Sox2 is inhibited, differentiation is induced in mouse embryonic stem cells. Further, the Sox2-binding site, which is found in promoter regions of several lower-stream genes of Sox2, is often present adjacent to the Oct-4- or Nanog-binding site. Thus, the interaction between Sox2 and Oct-4 transcription factors enables the reprogrammed stem cells to maintain an undifferentiated state and to show characteristics of embryonic stem cells.

c-Myc, a helix-loop-helix/leucine zipper transcription factor, is an oncogene involved in various intracellular functions such as cell growth, differentiation, proliferation, apoptosis, and transformation into cancerous cells. c-Myc is a lower stream gene in a signal delivery mechanism by LIF(leukaemia inhibitory factor)/STAT3 and Wnt, which is a main mechanism for pluripotency maintenance. c-Myc transcription factor prevents the p21 gene from inhibiting cell proliferation by being activated by the Klf4 gene in the reprogrammed stem cells. In addition, c-Myc changes the structure of chromatin by binding to the Myc recognition site present on the genome and attracting the histone acetylase complex so that Oct-4 and Sox2 can successfully bind to the target genes.

Klf4 is a transcription factor belonging to the Kruppel-like factor (Klf) family. It was used in the production of murine and human reprogrammed stem cells by the Yamanaka group and the Jaenisch group. However, given that the Thomson group reported the establishment of human reprogrammed stem cells without Klf4, this transcription factor may not be essential for the production of human reprogrammed stem cells. Klf4 is characterized by its Kruppel-type zinc-finger and participates in inhibition of growth, thereby regulating a cell cycle. It was reported that Klf4, like c-Myc, is a lower-stream gene of STAT3 in embryonic stem cells, and its overexpression maintains Oct-4 expression, thereby inhibiting differentiation of mouse embryonic stem cells. In addition, Klf4, together with Sox2, is responsible for the regulation of expression of Lefty1, which is known as a lower-stream gene of Oct-4 in embryonic stem cells and inhibits the function of the p53 tumor suppressor gene. Thus, it is thought that Klf4 indirectly enhances the activity of Nanog, which is known as an embryo-specific gene and suppresses apoptosis induced by c-Myc to induce production of reprogrammed stem cells. It has been reported that Klf2, as well as Klf4, can be employed in the production of human reprogrammed stem cells. Further, other relevant genes, i.e., Klf1 and Klf5, can also be used in the production of human reprogrammed stem cells, although they may show lower efficiency than Klf4 or Klf2.

Nanog is an embryo-specific gene which does not act through the LIF/STAT3 mechanism. Like Oct-4 or Sox2, this independent transcription factor is involved in the maintenance of pluripotency of embryonic stem cells and suppresses GATA4 and GATA6 transcription factors responsible for differentiation into endoderm.

Lastly, LIN28, which is an mRNA-binding protein, is expressed in embryonic stem cells and embryonic tumor cells and is known to be involved in differentiation and proliferation. Although Nanog and LIN28 are not indispensible in the production of reprogrammed stem cells, the Thomson group demonstrated the establishment of reprogrammed stem cells using these two genes.

In many studies currently conducted to establish reprogrammed stem cells using the six reprogramming transcription factors, the key issue is in vivo transduction of the transcription factors. Thus, relevant techniques are being intensively studied. Among them, transduction with a virus vector is commonly used to deliver the reprogramming transcription factors. However, the viral vector transduction technique accompanies a side effect of tumor generation. It has been reported that when a retrovirus or lentivirus encoding any of the six reprogramming transcription factors (Oct4, Sox2, c-Myc, Klf4, Nanog, and Lin28) is inserted into a somatic gene, the gene is mutated due to integration of the external genes and the inserted c-Myc oncogene generates teratoma in 25% of the chimera generated from the reprogrammed cells.

Such a method of transducing a reprogramming transcription factor using a virus or plasmid is limited in cellular therapy due to possibilities of mutagenesis and oncogenesis. However, the method of transducing a cell-permeable reprogramming transcription factor using a protein transduction method is a safe method free of insertion of external genes. Accordingly, the inventors of the present invention aimed at producing a cell permeable reprogramming transcription factor (Cell Permeable CP-TFs) by fusing a “MTD” to a reprogramming transcription factor recombinant protein.

Meanwhile, small molecules of synthetic compounds or compounds existing in nature can be transported into cells, whereas macromolecules, such as proteins, peptides, and nucleic acids, cannot due to the large molecular weight. It is widely understood that macromolecules larger than 500 kDa are incapable of penetrating a plasma membrane, i.e., the lipid bilayer structure, of living cells. In order to overcome this problem, “macromolecule intracellular transduction technology (MITT)” was developed, which allows the delivery of therapeutically effective macromolecules into cells, thereby facilitating the development of new biomolecular drugs using peptides, proteins, and genetic materials per se. According to this technology, a target macromolecule is fused to a “hydrophobic MTD” and other various transporters to cells, and is synthesized or expressed and purified in the form of a recombinant protein. Then, it is administered to a subject and can be delivered to a target site rapidly and accurately where its therapeutic effect is effectively shown. See also Korean Patent Application No. 10-2009-7017564; U.S. patent application Ser. No. 12/524,935; Canada Patent Application No. 2,676,797; Chinese Patent Application No. 200880003468.9; Australia Patent Application No. 2008211854; India Patent Application No. 5079/CHENP/2009; European Patent Application No. 08712219.8; and Japanese Patent Application No. 2009-547177 (which claim priority based on U.S. Provisional Patent Application No. 60/887,060). As such, MTDs allow the transport of many impermeable materials into the cells by being fused to peptides, proteins, DNA, RNA, synthetic compounds, and the like.

Accordingly, the inventors of the present invention have developed reprogramming transcription factor recombinant proteins imparted with cell permeability by fusing six types of transcription factors (Nanog, Oct4, Sox2, Klf4, c-Myc, and Lin28) to MTDs. The inventors found that these recombinant proteins can maintain pluripotency, self-renewal, and the undifferentiated state of human somatic cells, thereby inducing reprogramming safely without insertion of exogenous genes, and contemplated the present invention.

### SUMMARY OF THE INVENTION

The objective of the present invention is to provide a cell permeable reprogramming transcription factor recombinant protein which can be introduced into a cell with high efficiency and can induce pluripotency of somatic cells, whereby induced pluripotent stem cells are established.

In order to achieve the objective above, the present invention provides a cell permeable reprogramming transcription factor recombinant protein by fusing a MTD to a reprogramming transcription factor. The recombinant protein can then introduce the reprogramming transcription factor into a cell with high efficiency.

The present invention also provides a polynucleotide encoding the cell permeable reprogramming transcription factor recombinant protein above.

The present invention further provides an expression vector comprising the polynucleotide above and a transformant prepared with such expression vector. In addition, the present invention provides a method of producing a cell permeable reprogramming transcription factor recombinant protein comprising culturing the transformant above.

Lastly, the present invention provides a cell permeable reprogramming transcription factor containing the cell permeable reprogramming transcription factor recombinant protein above as an active ingredient, which can establish induced pluripotent stem cells safely in a high yield.

### BRIEF DESCRIPTION OF THE DRAWINGS

FIG. 1 illustrates the structure of the reprogramming transcription factor recombinant proteins according to the present invention in which one of JO-84 and JO-86 MTDs is fused to each of the reprogramming transcription factors (Nanog, Oct4, Sox2, Klf4, cMyc, and Lin28).

FIG. 2 shows the results of examining the expression of the cell permeable reprogramming transcription factor recombinant proteins according to the present invention in the presence (+) and absence (−) of IPTG, a protein expression inducer.

FIG. 3 is a schematic diagram illustrating the process of making pluripotent stem cells and shows the result of alkaline phosphatase staining of cell colonies, which was obtained by treating human dermal fibroblast (HDF) cells with the recombinant protein according to the present invention, as illustrated in FIG. 3.

FIG. 4 shows the result of staining of the genes expressed specifically in the nucleus of stem cells (Oct4 and Nanog) in the colonies obtained by treating human dermal fibroblast (HDF) cells with the recombinant protein according to the present invention, as illustrated in FIG. 3.

FIG. 5 shows the result of flow cytometry analysis of the expression of a specific marker (stage-specific embryonic antigen-3: SSEA-3) on the surface of induced pluripotent stem cells obtained by treating human dermal fibroblast (HDF) cells with the recombinant protein according to the present invention, as illustrated in FIG. 3.

FIG. 6 is the result of RT-PCR analysis showing the RNA expression of a stem cell specific marker in the induced pluripotent stem cells obtained by treating human dermal fibroblast (HDF) cells with the recombinant protein according to the present invention, as illustrated in FIG. 3.

FIG. 7 is a schematic diagram illustrating the process of making the induced pluripotent stem cells according to the present invention and a microscopic photograph visualizing the colony of the induced pluripotent stem cells obtained by treating human dermal fibroblast (HDF) cells with the recombinant protein according to the present invention, as illustrated in FIG. 7.

FIG. 8 shows the result of staining of the genes expressed specifically in the nucleus of stem cells (Oct4 and Nanog) in the cell colonies obtained by treating human dermal fibroblast (HDF) cells with the recombinant protein according to the present invention, as illustrated in FIG. 7.

FIG. 9 shows the result of flow cytometry analysis of the expression of two stem cell specific markers, i.e., SSEA-3 (Stage-Specific Embryonic Antigen-3) and TRA-1-60 (Tumor Rejection Antigen-1-60), on the surface of the pluripotent stem cells obtained by treating human dermal fibroblast (HDF) cells with the recombinant protein according to the present invention, as illustrated in FIG. 7.

FIG. 10 illustrates the structure of the reprogramming transcription factor recombinant proteins in which one of JO-10, JO-52, JO-132, JO-145, JO-173, and JO-181 MTDs is fused to each of the reprogramming transcription factors (Nanog, Oct4, Sox2, Klf4, cMyc, and Lin28) according to the present invention.

FIG. 11 is the result of examining the expression of the reprogramming transcription factor recombinant proteins according to the present invention which had been subjected to a codon optimization to optimize the expression in the presence (+) and absence (−) of IPTG, a protein expression inducer.

### DETAILED DESCRIPTION OF THE INVENTION

The present invention provides cell permeable Nanog, Oct4, Sox2, Klf4, cMyc, and Lin28 recombinant proteins (CP-Nanog, CP-Oct4, CP-Sox2, CP-Klf4, CP-cMyc, and CP-Lin28) in which a MTD is fused to the reprogramming transcription factor, whereby the reprogramming transcription factor becomes cell permeable and can be introduced into a cell with high efficiency, and polynucleotides encoding the same.

The present invention uses proteins that acquired cell permeability by fusion of MTDs to Nanog, Oct4, Sox2, Klf4, cMyc, and Lin28 reprogramming transcription factors.

Among those, a recombinant protein in which a full length Nanog is fused to a nuclear localization sequence (NLS), i.e., NLS-Nanog (NNanog), has an amino acid sequence represented by SEQ ID NO: 20. NLS-Nanog can be encoded by, for example, a polynucleotide having a nucleotide sequence represented by SEQ ID NO: 19.

The full length Nanog recombinant proteins prepared by using a JO-84 MTD (MTD₈₄) may include the following:

NLS-MTD₈₄-Nanog (NM₈₄Nanog) has an amino acid sequence represented by SEQ ID NO: 22, and can be encoded by, for example, a polynucleotide having a nucleotide sequence represented by SEQ ID NO: 21;

NLS-Nanog-MTD₈₄ (NNanogM₈₄) has an amino acid sequence represented by SEQ ID NO: 24, and can be encoded by, for example, a polynucleotide having a nucleotide sequence represented by SEQ ID NO: 23; and

NLS-MTD₈₄-Nanog-MTD₈₄ (NM₈₄NanogM₈₄) has an amino acid sequence represented by SEQ ID NO: 26, and can be encoded by, for example, a polynucleotide having a nucleotide sequence represented by SEQ ID NO: 25.

The full-length Nanog recombinant proteins prepared by using a JO-86 MTD (MTD₈₆) may include the following:

NLS-MTD₈₆-Nanog (NM₈₆Nanog) has an amino acid sequence represented by SEQ ID NO: 28, and can be encoded by, for example, a polynucleotide having a nucleotide sequence represented by SEQ ID NO: 27;

NLS-Nanog-MTD₈₆ (NNanogM₈₆) has an amino acid sequence represented by SEQ ID NO: 30, and can be encoded by, for example, a polynucleotide having a nucleotide sequence represented by SEQ ID NO: 29; and

NLS-MTD₈₆-Nanog-MTD₈₆ (NM₈₆NanogM₈₆) has an amino acid sequence represented by SEQ ID NO: 32, and can be encoded by, for example, a polynucleotide having a nucleotide sequence represented by SEQ ID NO: 31.

In addition, the Nanog recombinant protein described above may further comprise a His-tag for easy isolation and purification.

His-NLS-Nanog (HNNanog) has an amino acid sequence represented by SEQ ID NO: 34, and can be encoded by, for example, a polynucleotide having a nucleotide sequence represented by SEQ ID NO: 33.

The full-length Nanog recombinant proteins prepared by using a JO-84 MTD (MTD₈₄) may include the following:

His-NLS-MTD₈₄-Nanog (HNM₈₄Nanog) has an amino acid sequence represented by SEQ ID NO: 36, and can be encoded by, for example, a polynucleotide having a nucleotide sequence represented by SEQ ID NO: 35;

His-NLS-Nanog-MTD₈₄ (HNNanogM₈₄) has an amino acid sequence represented by SEQ ID NO: 38, and can be encoded by, for example, a polynucleotide having a nucleotide sequence represented by SEQ ID NO: 37; and

His-NLS-MTD₈₄-Nanog-MTD₈₄ (HNM₈₄NanogM₈₄) has an amino acid sequence represented by SEQ ID NO: 40, and can be encoded by, for example, a polynucleotide having a nucleotide sequence represented by SEQ ID NO: 39.

The full-length Nanog recombinant proteins prepared by using a JO-86 MTD (MTD₈₆) may include the following:

His-NLS-MTD₈₆-Nanog (HNM₈₆Nanog) has an amino acid sequence represented by SEQ ID NO: 42, and can be encoded by, for example, a polynucleotide having a nucleotide sequence represented by SEQ ID NO: 41;

His-NLS-Nanog-MTD₈₆ (HNNanogM₈₆) has an amino acid sequence represented by SEQ ID NO: 44, and can be encoded by, for example, a polynucleotide having a nucleotide sequence represented by SEQ ID NO: 43; and

His-NLS-MTD₈₆-Nanog-MTD₈₆ (HNM₈₆NanogM₈₆) has an amino acid sequence represented by SEQ ID NO: 46, and can be encoded by, for example, a polynucleotide having a nucleotide sequence represented by SEQ ID NO: 45.

Further, His-NLS-MTD₁₀-Nanog (HNM₁₀Nanog) has an amino acid sequence represented by SEQ ID NO: 237, and can be encoded by, for example, a polynucleotide having a nucleotide sequence represented by SEQ ID NO: 236.

The full-length Oct4 recombinant proteins fused to an NLS include NLS-Oct4 (NOct4), which has an amino acid sequence represented by SEQ ID NO: 48, and can be encoded by, for example, a polynucleotide having a nucleotide sequence represented by SEQ ID NO: 47.

The full-length Oct4 recombinant proteins prepared by using a JO-84 MTD (MTD₈₄) may include the following:

NLS-MTD₈₄-Oct4 (NM₈₄Oct4) has an amino acid sequence represented by SEQ ID NO: 50, and can be encoded by, for example, a polynucleotide having a nucleotide sequence represented by SEQ ID NO: 49;

NLS-Oct4-MTD₈₄ (NOct4M₈₄) has an amino acid sequence represented by SEQ ID NO: 52, and can be encoded by, for example, a polynucleotide having a nucleotide sequence represented by SEQ ID NO: 51; and

NLS-MTD₈₄-Oct4-MTD₈₄ (NM₈₄Oct4M₈₄) has an amino acid sequence represented by SEQ ID NO: 54, and can be encoded by, for example, a polynucleotide having a nucleotide sequence represented by SEQ ID NO: 53.

The full-length Oct4 recombinant proteins prepared by using a JO-86 MTD (MTD₈₆) may include the following:

NLS-MTD₈₆-Oct4 (NM₈₆Oct4) has an amino acid sequence represented by SEQ ID NO: 56, and can be encoded by, for example, a polynucleotide having a nucleotide sequence represented by SEQ ID NO: 55;

NLS-Oct4MTD₈₆ (NOct4M₈₆) has an amino acid sequence represented by SEQ ID NO: 58, and can be encoded by, for example, a polynucleotide having a nucleotide sequence represented by SEQ ID NO: 57; and

NLS-MTD₈₆-Oct4-MTD₈₆ (NM₈₆Oct4M₈₆) has an amino acid sequence represented by SEQ ID NO: 60, and can be encoded by, for example, a polynucleotide having a nucleotide sequence represented by SEQ ID NO: 59.

In addition, the Oct4 recombinant protein described above may further comprise a His-tag for easy isolation and purification.

His-NLS-Oct4 (HNOct4) has an amino acid sequence represented by SEQ ID NO: 62, and can be encoded by, for example, a polynucleotide having a nucleotide sequence represented by SEQ ID NO: 61.

The full-length Oct4 recombinant proteins prepared by using a JO-84 MTD (MTD₈₄) may include the following:

His-NLS-MTD₈₄-Oct4 (HNM₈₄Oct4) has an amino acid sequence represented by SEQ ID NO: 64, and can be encoded by, for example, a polynucleotide having a nucleotide sequence represented by SEQ ID NO: 63;

His-NLS-Oct4-MTD₈₄ (HNOct4M₈₄) has an amino acid sequence represented by SEQ ID NO: 66, and can be encoded by, for example, a polynucleotide having a nucleotide sequence represented by SEQ ID NO: 65; and

His-NLS-MTD₈₄-Oct4-MTD₈₄ (HNM₈₄Oct4M₈₄) has an amino acid sequence represented by SEQ ID NO: 68, and can be encoded by, for example, a polynucleotide having a nucleotide sequence represented by SEQ ID NO: 67.

The full-length Oct4 recombinant proteins prepared by using a JO-86 MTD (MTD₈₆) may include the following:

His-NLS-MTD₈₆-Oct4 (HNM₈₆Oct4) has an amino acid sequence represented by SEQ ID NO: 70, and can be encoded by, for example, a polynucleotide having a nucleotide sequence represented by SEQ ID NO: 69;

His-NLS-Oct4-MTD₈₆ (HNOct4M₈₆) has an amino acid sequence represented by SEQ ID NO: 72, and can be encoded by, for example, a polynucleotide having a nucleotide sequence represented by SEQ ID NO: 71; and

His-NLS-MTD₈₆-Oct4-MTD₈₆ (HNM₈₆Oct4M₈₆) has an amino acid sequence represented by SEQ ID NO: 74, and can be encoded by, for example, a polynucleotide having a nucleotide sequence represented by SEQ ID NO: 73.

Further, His-NLS-MTD₅₂-Oct4 (HNM₅₂Oct4) has an amino acid sequence represented by SEQ ID NO: 240, and can be encoded by, for example, a polynucleotide having a nucleotide sequence represented by SEQ ID NO: 239.

The full-length Sox2 recombinant proteins fused to an NLS include NLS-Sox2 (NSox2), which has an amino acid sequence represented by SEQ ID NO: 76, and can be encoded by, for example, a polynucleotide having a nucleotide sequence represented by SEQ ID NO: 75.

The full-length Sox2 recombinant proteins prepared by using a JO-84 MTD (MTD₈₄) may include the following:

NLS-MTD₈₄-Sox2 (NM₈₄Sox2) has an amino acid sequence represented by SEQ ID NO: 78, and can be encoded by, for example, a polynucleotide having a nucleotide sequence represented by SEQ ID NO: 77;

NLS-Sox2-MTD₈₄ (NSox2M₈₄) has an amino acid sequence represented by SEQ ID NO: 80, and can be encoded by, for example, a polynucleotide having a nucleotide sequence represented by SEQ ID NO: 79; and

NLS-MTD₈₄-Sox2-MTD₈₄ (NM₈₄Sox2M₈₄) has an amino acid sequence represented by SEQ ID NO: 82, and can be encoded by, for example, a polynucleotide having a nucleotide sequence represented by SEQ ID NO: 81.

The full-length Sox2 recombinant proteins prepared by using a JO-86 MTD (MTD₈₆) may include the following:

NLS-MTD₈₆-Sox2 (NM₈₆Sox2) has an amino acid sequence represented by SEQ ID NO: 84, and can be encoded by, for example, a polynucleotide having a nucleotide sequence represented by SEQ ID NO: 83;

NLS-Sox2-MTD₈₆ (NSox2M₈₆) has an amino acid sequence represented by SEQ ID NO: 86, and can be encoded by, for example, a polynucleotide having a nucleotide sequence represented by SEQ ID NO: 85; and

NLS-MTD₈₆-Sox2-MTD₈₆ (NM₈₆Sox2M₈₆) has an amino acid sequence represented by SEQ ID NO: 88, and can be encoded by, for example, a polynucleotide having a nucleotide sequence represented by SEQ ID NO: 87.

In addition, the Sox2 recombinant proteins described above may further comprise a His-tag for easy isolation and purification.

His-NLS-Sox2 (HNSox2) has an amino acid sequence represented by SEQ ID NO: 90, and can be encoded by, for example, a polynucleotide having a nucleotide sequence represented by SEQ ID NO: 89.

The full-length Sox2 recombinant proteins prepared by using a JO-84 MTD (MTD₈₄) may include the following:

His-NLS-MTD₈₄-Sox2 (HNM₈₄Sox2) has an amino acid sequence represented by SEQ ID NO: 92, and can be encoded by, for example, a polynucleotide having a nucleotide sequence represented by SEQ ID NO: 91;

His-NLS-Sox2-MTD₈₄ (HNSox2M₈₄) has an amino acid sequence represented by SEQ ID NO: 94, and can be encoded by, for example, a polynucleotide having a nucleotide sequence represented by SEQ ID NO: 93; and

His-NLS-MTD₈₄-Sox2-MTD₈₄ (HNM₈₄Sox2M₈₄) has an amino acid sequence represented by SEQ ID NO: 96, and can be encoded by, for example, a polynucleotide having a nucleotide sequence represented by SEQ ID NO: 95.

The full-length Sox2 recombinant proteins prepared by using a JO-86 MTD (MTD₈₆) may include the following:

His-NLS-MTD₈₆-Sox2 (HNM₈₆Sox2) has an amino acid sequence represented by SEQ ID NO: 98, and can be encoded by, for example, a polynucleotide having a nucleotide sequence represented by SEQ ID NO: 97;

His-NLS-Sox2-MTD₈₆ (HNSox2M₈₆) has an amino acid sequence represented by SEQ ID NO: 100, and can be encoded by, for example, a polynucleotide having a nucleotide sequence represented by SEQ ID NO: 99; and

His-NLS-MTD₈₆-Sox2-MTD₈₆ (HNM₈₆Sox2M₈₆) has an amino acid sequence represented by SEQ ID NO: 102, and can be encoded by, for example, a polynucleotide having a nucleotide sequence represented by SEQ ID NO: 101.

Further, His-NLS-MTD₁₈₁-Sox2 (HNM₁₈₁Sox2) has an amino acid sequence represented by SEQ ID NO: 243, and can be encoded by, for example, a polynucleotide having a nucleotide sequence represented by SEQ ID NO: 242.

The full length Klf4 recombinant proteins fused to an NLS include NLS-Klf4 (NKlf4), which has an amino acid sequence represented by SEQ ID NO: 104, and can be encoded by, for example, a polynucleotide having a nucleotide sequence represented by SEQ ID NO: 103.

The full length Klf4 recombinant proteins prepared by using a JO-84 MTD (MTD₈₄) may include the following:

NLS-MTD₈₄-Klf4 (NM₈₄Klf4) has an amino acid sequence represented by SEQ ID NO: 106, and can be encoded by, for example, a polynucleotide having a nucleotide sequence represented by SEQ ID NO: 105;

NLS-Klf4-MTD₈₄ (NKlf4M₈₄) has an amino acid sequence represented by SEQ ID NO: 108, and can be encoded by, for example, a polynucleotide having a nucleotide sequence represented by SEQ ID NO: 107; and

NLS-MTD₈₄-Klf4-MTD₈₄ (NM₈₄Klf4M₈₄) has an amino acid sequence represented by SEQ ID NO: 110, and can be encoded by, for example, a polynucleotide having a nucleotide sequence represented by SEQ ID NO: 109.

The full length Klf4 recombinant proteins prepared by using a JO-86 MTD (MTD₈₆) may include the following:

NLS-MTD₈₆-Klf4 (NM₈₆Klf4) has an amino acid sequence represented by SEQ ID NO: 112, and can be encoded by, for example, a polynucleotide having a nucleotide sequence represented by SEQ ID NO: 111;

NLS-Klf4-MTD₈₆ (NKlf4M₈₆) has an amino acid sequence represented by SEQ ID NO: 114, and can be encoded by, for example, a polynucleotide having a nucleotide sequence represented by SEQ ID NO: 113; and

NLS-MTD₈₆-Klf4-MTD₈₆ (NM₈₆Klf4M₈₆) has an amino acid sequence represented by SEQ ID NO: 116, and can be encoded by, for example, a polynucleotide having a nucleotide sequence represented by SEQ ID NO: 115.

In addition, the Klf4 recombinant proteins described above may further comprise a His-tag for easy isolation and purification.

His-NLS-Klf4 (HNKlf4) has an amino acid sequence represented by SEQ ID NO: 118, and can be encoded by, for example, a polynucleotide having a nucleotide sequence represented by SEQ ID NO: 117.

The full length Klf4 recombinant proteins prepared by using a JO-84 MTD (MTD₈₄) may include the following:

His-NLS-MTD₈₄-Klf4 (HNM₈₄Klf4) has an amino acid sequence represented by SEQ ID NO: 120, and can be encoded by, for example, a polynucleotide having a nucleotide sequence represented by SEQ ID NO: 119;

His-NLS-Klf4-MTD₈₄ (HNKlf4M₈₄) has an amino acid sequence represented by SEQ ID NO: 122, and can be encoded by, for example, a polynucleotide having a nucleotide sequence represented by SEQ ID NO: 121; and

His-NLS-MTD₈₄-Klf4-MTD₈₄ (HNM₈₄Klf4M₈₄) has an amino acid sequence represented by SEQ ID NO: 124, and can be encoded by, for example, a polynucleotide having a nucleotide sequence represented by SEQ ID NO: 123.

The full length Klf4 recombinant proteins prepared by using a JO-86 MTD (MTD₈₆) may include the following:

His-NLS-MTD₈₆-Klf4 (HNM₈₆Klf4) has an amino acid sequence represented by SEQ ID NO: 126, and can be encoded by, for example, a polynucleotide having a nucleotide sequence represented by SEQ ID NO: 125;

His-NLS-Klf4-MTD₈₆ (HNKlf4M₈₆) has an amino acid sequence represented by SEQ ID NO: 128, and can be encoded by, for example, a polynucleotide having a nucleotide sequence represented by SEQ ID NO: 127; and

His-NLS-MTD₈₆-Klf4-MTD₈₆ (HNM₈₆Klf4M₈₆) has an amino acid sequence represented by SEQ ID NO: 130, and can be encoded by, for example, a polynucleotide having a nucleotide sequence represented by SEQ ID NO: 129.

Further, His-NLS-MTD₁₇₃-Klf4 (HNM₁₇₃Klf4) has an amino acid sequence represented by SEQ ID NO: 246, and can be encoded by, for example, a polynucleotide having a nucleotide sequence represented by SEQ ID NO: 245.

The full length cMyc recombinant proteins fused to a NLS include NLS-cMyc (NcMyc), which has an amino acid sequence represented by SEQ ID NO: 132, and can be encoded by, for example, a polynucleotide having a nucleotide sequence represented by SEQ ID NO: 131.

The full length cMyc recombinant proteins prepared by using a JO-84 MTD (MTD₈₄) may include the following:

NLS-MTD₈₄-cMyc (NM₈₄cMyc) has an amino acid sequence represented by SEQ ID NO: 134, and can be encoded by, for example, a polynucleotide having a nucleotide sequence represented by SEQ ID NO: 133;

NLS-cMyc-MTD₈₄ (NcMycM₈₄) has an amino acid sequence represented by SEQ ID NO: 136, and can be encoded by, for example, a polynucleotide having a nucleotide sequence represented by SEQ ID NO: 135; and

NLS-MTD₈₄-cMyc-MTD₈₄ (NM₈₄cMycM₈₄) has an amino acid sequence represented by SEQ ID NO: 138, and can be encoded by, for example, a polynucleotide having a nucleotide sequence represented by SEQ ID NO: 137.

The full length cMyc recombinant proteins prepared by using a JO-86 MTD (MTD₈₆) may include the following:

NLS-MTD₈₆-cMyc (NM₈₆cMyc) has an amino acid sequence represented by SEQ ID NO: 140, and can be encoded by, for example, a polynucleotide having a nucleotide sequence represented by SEQ ID NO: 139;

NLS-cMyc-MTD₈₆ (NcMycM₈₆) has an amino acid sequence represented by SEQ ID NO: 142, and can be encoded by, for example, a polynucleotide having a nucleotide sequence represented by SEQ ID NO: 141; and

NLS-MTD₈₆-cMyc-MTD₈₆ (NM₈₆cMycM₈₆) has an amino acid sequence represented by SEQ ID NO: 144, and can be encoded by, for example, a polynucleotide having a nucleotide sequence represented by SEQ ID NO: 143.

In addition, the cMyc recombinant proteins described above may further comprise a His-tag for easy isolation and purification.

His-NLS-cMyc (HNcMyc) has an amino acid sequence represented by SEQ ID NO: 146, and can be encoded by, for example, a polynucleotide having a nucleotide sequence represented by SEQ ID NO: 145.

The full length the cMyc recombinant proteins prepared by using a JO-84 MTD (MTD₈₄) may include the following:

His-NLS-MTD₈₄-cMyc (HNM₈₄cMyc) has an amino acid sequence represented by SEQ ID NO: 148, and can be encoded by, for example, a polynucleotide having a nucleotide sequence represented by SEQ ID NO: 147;

His-NLS-cMyc-MTD₈₄ (HNcMycM₈₄) has an amino acid sequence represented by SEQ ID NO: 150, and can be encoded by, for example, a polynucleotide having a nucleotide sequence represented by SEQ ID NO: 149; and

His-NLS-MTD₈₄-cMyc-MTD₈₄ (HNM₈₄cMycM₈₄) has an amino acid sequence represented by SEQ ID NO: 152, and can be encoded by, for example, a polynucleotide having a nucleotide sequence represented by SEQ ID NO: 151.

The full length cMyc recombinant proteins prepared by using a JO-86 MTD (MTD₈₆) may include the following:

His-NLS-MTD₈₆-cMyc (HNM₈₆cMyc) has an amino acid sequence represented by SEQ ID NO: 154, and can be encoded by, for example, a polynucleotide having a nucleotide sequence represented by SEQ ID NO: 153;

His-NLS-cMyc-MTD₈₆ (HNcMycM₈₆) has an amino acid sequence represented by SEQ ID NO: 156, and can be encoded by, for example, a polynucleotide having a nucleotide sequence represented by SEQ ID NO: 155; and

His-NLS-MTD₈₆-cMyc-MTD₈₆ (HNM₈₆cMycM₈₆) has an amino acid sequence represented by SEQ ID NO: 158, and can be encoded by, for example, a polynucleotide having a nucleotide sequence represented by SEQ ID NO: 157.

Further, His-NLS-MTD₁₄₅-cMyc (HNM₁₄₅cMyc) has an amino acid sequence represented by SEQ ID NO: 249, and can be encoded by, for example, a polynucleotide having a nucleotide sequence represented by SEQ ID NO: 248.

The full length Lin28 recombinant proteins fused to an NLS may include NLS-Lin28 (NLin28), which has an amino acid sequence represented by SEQ ID NO: 160, and can be encoded by, for example, a polynucleotide having a nucleotide sequence represented by SEQ ID NO: 159.

The full length Lin28 recombinant proteins prepared by using a JO-84 MTD (MTD₈₄) may include the following:

NLS-MTD₈₄-Lin28 (NM₈₄Lin28) has an amino acid sequence represented by SEQ ID NO: 162, and can be encoded by, for example, a polynucleotide having a nucleotide sequence represented by SEQ ID NO: 161;

NLS-Lin28-MTD₈₄ (NLin28M₈₄) has an amino acid sequence represented by SEQ ID NO: 164, and can be encoded by, for example, a polynucleotide having a nucleotide sequence represented by SEQ ID NO: 163; and

NLS-MTD₈₄-Lin28-MTD₈₄ (NM₈₄Lin28M₈₄) has an amino acid sequence represented by SEQ ID NO: 166, and can be encoded by, for example, a polynucleotide having a nucleotide sequence represented by SEQ ID NO: 165.

The full length Lin28 recombinant proteins prepared by using a JO-86 MTD (MTD₈₆) may include the following:

NLS-MTD₈₆-Lin28 (NM₈₆Lin28) has an amino acid sequence represented by SEQ ID NO: 168, and can be encoded by, for example, a polynucleotide having a nucleotide sequence represented by SEQ ID NO: 167;

NLS-Lin28-MTD₈₆ (NLin28M₈₆) has an amino acid sequence represented by SEQ ID NO: 170, and can be encoded by, for example, a polynucleotide having a nucleotide sequence represented by SEQ ID NO: 169; and

NLS-MTD₈₆-Lin28-MTD₈₆ (NM₈₆Lin28M₈₆) has an amino acid sequence represented by SEQ ID NO: 172, and can be encoded by, for example, a polynucleotide having a nucleotide sequence represented by SEQ ID NO: 171.

In addition, the Lin28 recombinant proteins described above may further comprise a His-tag for easy isolation and purification.

His-NLS-Lin28 (HNLin28) has an amino acid sequence represented by SEQ ID NO: 174, and can be encoded by, for example, a polynucleotide having a nucleotide sequence represented by SEQ ID NO: 173.

The full length Lin28 recombinant proteins prepared by using a JO-84 MTD (MTD₈₄) may include the following:

His-NLS-MTD₈₄-Lin28 (HNM₈₄Lin28) has an amino acid sequence represented by SEQ ID NO: 176, and can be encoded by, for example, a polynucleotide having a nucleotide sequence represented by SEQ ID NO: 175;

His-NLS-Lin28-MTD₈₄ (HNLin28M₈₄) has an amino acid sequence represented by SEQ ID NO: 178, and can be encoded by, for example, a polynucleotide having a nucleotide sequence represented by SEQ ID NO: 177; and

His-NLS-MTD₈₄-Lin28-MTD₈₄ (HNM₈₄Lin28M₈₄) has an amino acid sequence represented by SEQ ID NO: 180, and can be encoded by, for example, a polynucleotide having a nucleotide sequence represented by SEQ ID NO: 179.

The full length the Lin28 recombinant proteins prepared by using a JO-86 MTD (MTD₈₆) may include the following:

His-NLS-MTD₈₆-Lin28 (HNM₈₆Lin28) has an amino acid sequence represented by SEQ ID NO: 182, and can be encoded by, for example, a polynucleotide having a nucleotide sequence represented by SEQ ID NO: 181;

His-NLS-Lin28-MTD₈₆ (HNLin28M₈₆) has an amino acid sequence represented by SEQ ID NO: 184, and can be encoded by, for example, a polynucleotide having a nucleotide sequence represented by SEQ ID NO: 183; and

His-NLS-MTD₈₆-Lin28-MTD₈₆ (HNM₈₆Lin28M₈₆) has an amino acid sequence represented by SEQ ID NO: 186, and can be encoded by, for example, a polynucleotide having a nucleotide sequence represented by SEQ ID NO: 185.

Further, His-NLS-MTD₁₃₂-Lin28 (HNM₁₃₂Lin28) has an amino acid sequence represented by SEQ ID NO: 252, and can be encoded by, for example, a polynucleotide having a nucleotide sequence represented by SEQ ID NO: 251.

The present invention is characterized by providing a reprogramming transcription factor, which is a macromolecule incapable of being introduced into a cell, with cell permeability by fusing it to a specific macromolecule transduction domain (hereinafter, “MTD”) by MITT. Thus, the reprogramming transcription factor can be transported into a cell with high efficiency. The MTD may be fused to either one or both termini of the reprogramming transcription factor. MITT utilizing a hydrophobic polypeptide MTD derived from a secreted protein enables a cargo protein to penetrate through cell membranes by treatment of the protein only. Thus, the protein can be delivered into the nucleus.

Using this effect, reprogramming transcription factors that are not expressed in somatic cells can be delivered into cells. Thus, induced pluripotent stem cells can be safely established without insertion of exogenous genetic materials, such as viruses and plasmids, as in prior art.

The present invention has developed cell permeable reprogramming transcription factor recombinant proteins that are constructed by fusing each of the reprogramming transcription factors to a peptide domain capable of transporting a macromolecule into a cell, i.e., MTD.

The term “cell permeable reprogramming transcription factor recombinant protein” used herein refers to a covalently linked complex comprising an MTD and the reprogramming transcription factor, where they are linked by genetic fusion or chemical coupling. The term “genetic fusion” used herein refers to a linear, covalent linkage of proteins generated through genetic expression of a polynucleotide (DNA sequence) molecule encoding the proteins.

Nanog, a transcription factor capable of reprogramming somatic cells having a nucleotide sequence represented by SEQ ID NO: 1 and an amino acid sequence represented by SEQ ID NO: 2, maintains pluripotency of embryonic cells and inhibits differentiation of cells into an endoderm. This reprogramming transcription factor is composed of a domain consisting of amino acid residues 1 to 306 of the amino acid sequence of SEQ ID NO: 2.

Oct4, a transcription factor capable of reprogramming somatic cells having a nucleotide sequence represented by SEQ ID NO: 3 and an amino acid sequence represented by SEQ ID NO: 4, maintains pluripotency and regulates self-renewal of stem cells. This reprogramming transcription factor is composed of a domain consisting of amino acid residues 1 to 361 of the amino acid sequence of SEQ ID NO: 4.

Sox2, a transcription factor capable of reprogramming somatic cells having a nucleotide sequence represented by SEQ ID NO: 5 and an amino acid sequence represented by SEQ ID NO: 6, maintains pluripotency and regulates the undifferentiated state of stem cells. This reprogramming transcription factor is composed of a domain consisting of amino acid residues 1 to 318 of the amino acid sequence of SEQ ID NO: 6.

Klf4 is a transcription factor capable of reprogramming somatic cells having a nucleotide sequence represented by SEQ ID NO: 7 and an amino acid sequence represented by SEQ ID NO: 8. This reprogramming transcription factor induces reprogramming by inhibiting proliferation and regulating cell cycle. Klf4 is composed of a domain consisting of amino acid residues 1 to 471 of the amino acid sequence of SEQ ID NO: 8.

cMyc is a transcription factor capable of reprogramming somatic cells having a nucleotide sequence represented by SEQ ID NO: 9 and an amino acid sequence represented by SEQ ID NO: 10. This reprogramming transcription factor induces reprogramming by regulating cell growth, proliferation, apoptosis, and cell cycle. cMyc is composed of a domain consisting of amino acid residues 1 to 456 of the amino acid sequence of SEQ ID NO: 10.

Lin28 is a transcription factor capable of reprogramming somatic cells having a nucleotide sequence represented by SEQ ID NO: 11 and an amino acid sequence represented by SEQ ID NO: 12. This reprogramming transcription factor functions to regulate differentiation and proliferation of stem cells. Lin28 is composed of a domain consisting of amino acid residues 1 to 210 of the amino acid sequence of SEQ ID NO: 12.

As the MTD capable of being fused to the six types of reprogramming transcription factors, a cell permeable peptide having a nucleotide sequence represented by SEQ ID NO: 15 and an amino acid sequence represented by SEQ ID NO: 16 may be used.

As the MTD capable of being fused to the six types of reprogramming transcription factors, a cell permeable peptide having a nucleotide sequence represented by SEQ ID NO: 17 and an amino acid sequence represented by SEQ ID NO: 18 may be used.

As the MTD capable of being fused to the Nanog reprogramming transcription factor, a cell permeable peptide having a nucleotide sequence represented by SEQ ID NO: 223 and an amino acid sequence represented by SEQ ID NO: 224 may be used.

As the MTD capable of being fused to the Oct4 reprogramming transcription factor, a cell permeable peptide having a nucleotide sequence represented by SEQ ID NO: 225 and an amino acid sequence represented by SEQ ID NO: 226 may be used.

As the MTD capable of being fused to the Sox2 reprogramming transcription factor, a cell permeable peptide having a nucleotide sequence represented by SEQ ID NO: 233 and an amino acid sequence represented by SEQ ID NO: 234 may be used.

As the MTD capable of being fused to the Klf4 reprogramming transcription factor, a cell permeable peptide having a nucleotide sequence represented by SEQ ID NO: 231 and an amino acid sequence represented by SEQ ID NO: 232 may be used.

As the MTD capable of being fused to the cMyc reprogramming transcription factor, a cell permeable peptide having a nucleotide sequence represented by SEQ ID NO: 229 and an amino acid sequence represented by SEQ ID NO: 230 may be used.

As the MTD capable of being fused to the Lin28 reprogramming transcription factor, a cell permeable peptide having a nucleotide sequence represented by SEQ ID NO: 227 and an amino acid sequence represented by SEQ ID NO: 228 may be used.

MTDs having any one of the amino acid sequences represented by SEQ ID NOS: 16, 18, 224, 226, 228, 230, 232, or 234 described above are cell permeable polypeptides capable of mediating the transport of a biologically active molecule, such as a polypeptide, a protein domain, or a full-length protein, across the cell membrane.

The MTDs according to the present invention are designed to include a hydrophobic region having cell membrane-targeting activity by forming a helix which is derived from a signal peptide comprising three regions, i.e., an N-terminal region, a hydrophobic region, and a C-terminal region containing a secreted protein cleavage site. These MTDs can directly penetrate the cell membrane, while avoiding any cell damage, and deliver a target protein into a cell for it to exhibit its desired function.

The MTDs having any one of the amino acid sequences represented by SEQ ID NOS: 16, 18, 224, 226, 228, 230, 232, or 234 to be fused to the reprogramming transcription factors according to the present invention are summarized in Table 1 below.

TABLE 1

| | | | | | | Cell |
|---|---|---|---|---|---|---|
| ID | Origin | Sequence | Length | Induction | Purification | Permeability |
| JO-84 | Streptomyces | LVAALLAVL | 9 | ++++ | ++++ | 1.9 |
| | coelicolor | | | | | |
| JO-86 | Mycobacterium | LAVLAAAP | 8 | ++++ | ++++ | 3.7 |
| | bovis | | | | | |
| JO-10 | Streptomyces | LGGAVVAAPVAAAVAP | 16 | ++++ | ++++ | 0.8 |
| | coelicolor | | | | | |
| JO-52 | Homo sapiens | PLLLLLPAL | 9 | ++++ | ++++ | 1.6 |
| JO-132 | Streptomyces | AVVVPAIVLAAP | 12 | ++ | ++ | 1.3 |
| | coelicolor | | | | | |
| JO-145 | Supravalvular | AAAPVLLLLL | 10 | ++++ | ++++ | 1.3 |
| | aortic stenosis | | | | | |
| JO-173 | Streptomyces | AVIPILAVP | 9 | ++++ | ++++ | 3.7 |
| | coelicolor | | | | | |
| JO-181 | Neisseria | AVLLLPAAA | 9 | ++++ | ++++ | 3.5 |
| | meningitidis | | | | | |

The cell permeable reprogramming transcription factor recombinant proteins according to the present invention may have a structure where one of the eight MTDs above is fused to either one or both termini of the reprogramming transcription factor, and optionally, a nuclear localization sequence (NLS) derived from SV40 large T antigen or a histidine-tag (His-Tag) affinity domain can be fused to one terminus of this fused construct for easy purification.

The NLS that can be fused to the reprogramming transcription factors may include, but is not limited to, a polypeptide having an amino acid sequence represented by SEQ ID NO: 14 or encoded by a nucleotide represented by SEQ ID NO: 13. Any known NLS may be used.

In one embodiment of the present invention, one of the following MTDs is used for fusion with a reprogramming transcription factor: a JO-84 MTD having the amino acid sequence represented by SEQ ID NO: 16, which is derived from Streptomyces coelicolor (hereinafter, “MTD₈₄”); a JO-86 MTD having the amino acid sequence represented by SEQ ID NO: 18, which is a secreted protein derived from Mycobacterium bovis (hereinafter, “MTD₈₆”); a JO-10 MTD having the amino acid sequence represented by SEQ ID NO: 224, which is derived from Streptomyces coelicolor (hereinafter, “MTD₁₀”); a JO-52 MTD having the amino acid sequence represented by SEQ ID NO: 226, which is derived from Homo Sapiens (hereinafter, “MTD₅₂”); a JO-132 MTD having the amino acid sequence represented by SEQ ID NO: 228, which is derived from Streptomyces coelicolor (hereinafter, “MTD₁₃₂”); a JO-145 MTD having the amino acid sequence represented by SEQ ID NO: 230, which is derived from Homo Sapiens Elastin (hereinafter, “MTD₁₄₅”); a JO-173 MTD having the amino acid sequence represented by SEQ ID NO: 232, which is derived from Streptomyces coelicolor (hereinafter, “MTD₁₇₃”); and a JO-181 MTD having the amino acid sequence represented by SEQ ID NO: 234, which is derived from Neisseria meningitidis (hereinafter, “MTD₁₈₁”).

In a preferred embodiment of the present invention, two full-length forms of reprogramming transcription factor recombinant proteins using a JO-84 MTD or a JO-86 MTD are designed for each MTD.

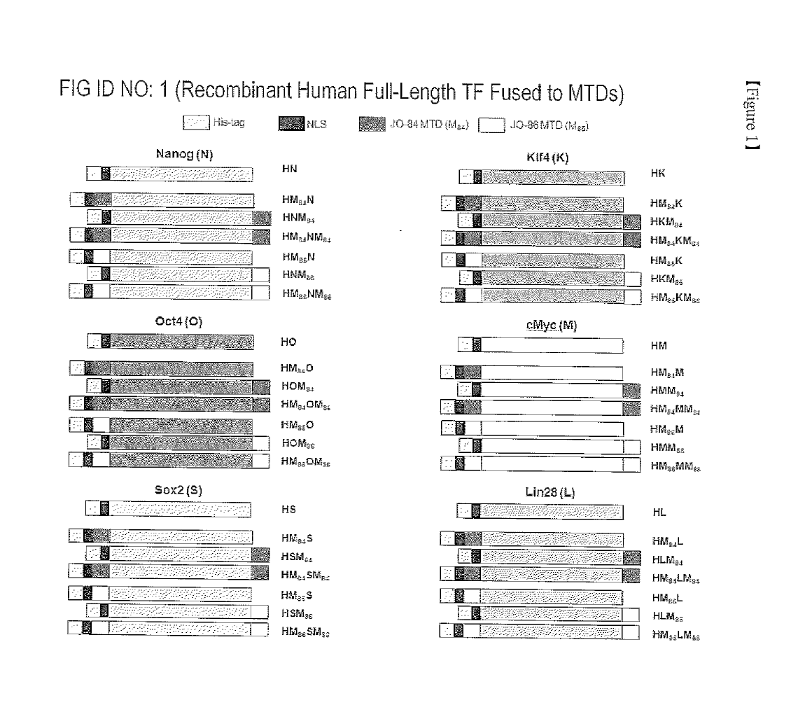

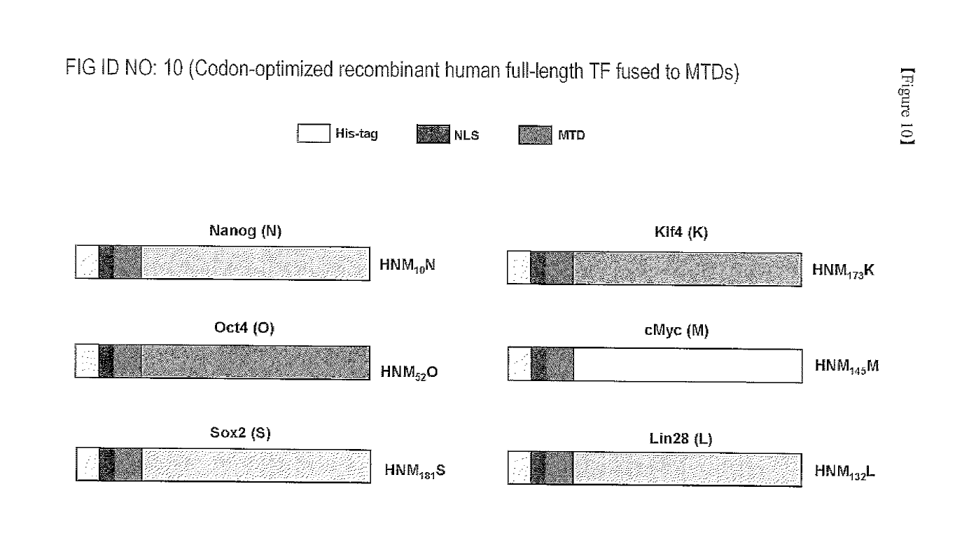

Referring to FIGS. 1 and 10, the Nanog recombinant protein according to the present invention may be:

1) His-NLS-MTD₈₄-Nanog (HNM₈₄Nanog), wherein His-NLS and a JO-84 MTD are fused to the N-terminus of Nanog;

2) His-NLS-Nanog-MTD₈₄ (HNNanogM₈₄), wherein His-NLS is fused to the N-terminus and a JO-84 MTD is fused to the C-terminus of Nanog;

3) His-NLS-MTD₈₄-Nanog-MTD₈₄ (HNM₈₄NanogM₈₄), wherein His-NLS-JO-84 MTD is fused to the N-terminus and a JO-84 MTD is fused to the C-terminus of Nanog;

4) His-NLS-MTD₈₆-Nanog (HNM₈₆Nanog), wherein His-NLS and a JO-86 MTD are fused to the N-terminus of Nanog;

5) His-NLS-Nanog-MTD₈₆ (HNNanogM₈₆), wherein His-NLS is fused to the N-terminus and a JO-86 MTD is fused to the C-terminus of Nanog;

6) His-NLS-MTD₈₆-Nanog-MTD₈₆ (HNM₈₆NanogM₈₆), wherein His-NLS-JO-86 MTD is fused to the N-terminus and a JO-86 MTD is fused to the C-terminus of Nanog; or

7) His-NLS-MTD₁₀-Nanog (HNM₁₀Nanog), wherein His-NLS-JO-10 MTD is fused to the N-terminus of Nanog.

Optionally, the recombinant proteins 1) to 7) above may not include His-Tag.

Referring to FIGS. 1 and 10, the Oct4 recombinant proteins of the present invention may be:

1) His-NLS-MTD₈₄-Oct4 (HNM₈₄Oct4), wherein His-NLS and JO-84 MTD are fused to the N-terminus of Oct4;

2) His-NLS-Oct4-MTD₈₄ (HNOct4M₈₄), wherein His-NLS is fused to the N-terminus and a JO-84 MTD is fused to the C-terminus of Oct4;

3) His-NLS-MTD₈₄-Oct4-MTD₈₄ (HNM₈₄Oct4M₈₄), wherein His-NLS-JO-84 MTD is fused to the N-terminus and a JO-84 MTD is fused to the C-terminus of Oct4;

4) His-NLS-MTD₈₆-Oct4 (HNM₈₆Oct4), wherein His-NLS and a JO-86 MTD are fused to the N-terminus of Oct4;

5) His-NLS-Oct4-MTD₈₆ (HNOct4M₈₆), wherein His-NLS is fused to the N-terminus and a JO-86 MTD is fused to the C-terminus of Oct4;

6) His-NLS-MTD₈₆-Oct4-MTD₈₆ (HNM₈₆Oct4M₈₆), wherein His-NLS-JO-86 MTD is fused to the N-terminus and a JO-86 MTD is fused to the C-terminus of Oct4; or

7) His-NLS-MTD₅₂-Oct4 (HNM₅₂Oct4), wherein His-NLS and a JO-52 MTD are fused to the N-terminus of Oct4.

Optionally, the recombinant proteins 1) to 7) above may not include His-Tag.

Referring to FIGS. 1 and 10, the Sox2 recombinant proteins of the present invention may be:

1) His-NLS-MTD₈₄-Sox2 (HNM₈₄Sox2), wherein a His-NLS and a JO-84 MTD are fused to the N-terminus of Sox2;

2) His-NLS-Sox2-MTD₈₄ (HNSox2M₈₄), wherein a His-NLS is fused to the N-terminus and a JO-84 MTD is fused to the C-terminus of Sox2;

3) His-NLS-MTD₈₄-Sox2-MTD₈₄ (HNM₈₄Sox2M₈₄), wherein a His-NLS-JO-84 MTD is fused to the N-terminus and a JO-84 MTD is fused to the C-terminus of Sox2;

4) His-NLS-MTD₈₆-Sox2 (HNM₈₆Sox2), wherein a His-NLS and a JO-86 MTD are fused to the N-terminus of Sox2;

5) His-NLS-Sox2-MTD₈₆ (HNSox2M₈₆), wherein a His-NLS is fused to the N-terminus and a JO-86 MTD is fused to the C-terminus of Sox2;

6) His-NLS-MTD₈₆-Sox2-MTD₈₆ (HNM₈₆Sox2M₈₆), wherein a His-NLS-JO-86 MTD is fused to the N-terminus and a JO-86 MTD is fused to the C-terminus of Sox2; or

7) His-NLS-MTD₁₈₁-Sox2 (HNM₁₈₁Sox2), wherein a His-NLS is fused to the N-terminus and a JO-181 MTD is fused to the C-terminus of Sox2.

Optionally, the recombinant proteins 1) to 7) above may not include His-Tag.

Referring to FIGS. 1 and 10, the Klf4 recombinant proteins of the present invention may be:

1) His-NLS-MTD₈₄-Klf4 (HNM₈₄Klf4), wherein a His-NLS and a JO-84 MTD are fused to the N-terminus of Klf4;

2) His-NLS-Klf4-MTD₈₄ (HNKlf4M₈₄), wherein a His-NLS is fused to the N-terminus and a JO-84 MTD is fused to the C-terminus of Klf4;

3) His-NLS-MTD₈₄-Klf4-MTD₈₄ (HNM₈₄Klf4M₈₄), wherein a His-NLS-JO-84 MTD is fused to the N-terminus and a JO-84 MTD is fused to the C-terminus of Klf4;

4) His-NLS-MTD₈₆-Klf4 (HNM₈₆Klf4), wherein a His-NLS and a JO-86 MTD are fused to the N-terminus of Klf4;

5) His-NLS-Klf4-MTD₈₆ (HNKlf4M₈₆), wherein a His-NLS is fused to the N-terminus and a JO-86 MTD is fused to the C-terminus of Klf4;

6) His-NLS-MTD₈₆-Klf4-MTD₈₆ (HNM₈₆Klf4M₈₆), wherein a His-NLS-JO-86 MTD is fused to the N-terminus and a JO-86 MTD is fused to the C-terminus of Klf4; or

7) His-NLS-MTD₁₇₃-Klf4 (HNM₁₇₃Klf4), wherein a His-NLS and a JO-173 MTD are fused to the N-terminus of Klf4.

Optionally, the recombinant proteins 1) to 7) above may not include His-Tag.

Referring to FIGS. 1 and 10, the cMyc recombinant proteins of the present invention may be:

1) His-NLS-MTD₈₄-cMyc (HNM₈₄cMyc), wherein a His-NLS and a JO-84 MTD are fused to the N-terminus of cMyc;

2) His-NLS-cMyc-MTD₈₄ (HNcMycM₈₄), wherein a His-NLS is fused to the N-terminus and a JO-84 MTD is fused to the C-terminus of cMyc;

3) His-NLS-MTD₈₄-cMyc-MTD₈₄ (HNM₈₄cMycM₈₄), wherein a His-NLS-JO-84 MTD is fused to the N-terminus and a JO-84 MTD is fused to the C-terminus of cMyc;

4) His-NLS-MTD₈₆-cMyc (HNM₈₆cMyc), wherein a His-NLS and a JO-86 MTD are fused to the N-terminus of cMyc,

5) His-NLS-cMyc-MTD₈₆ (HNcMycM₈₆), wherein a His-NLS is fused to the N-terminus and a JO-86 MTD is fused to the C-terminus of cMyc,

6) His-NLS-MTD₈₆-cMyc-MTD₈₆ (HNM₈₆cMycM₈₆), wherein a His-NLS-JO-86 MTD is fused to the N-terminus and a JO-86 MTD is fused to the C-terminus of cMyc, or

7) His-NLS-MTD₁₄₅-cMyc (HNM₁₄₅cMyc), wherein a His-NLS and a JO-145 MTD are fused to the N-terminus of cMyc.

Optionally, the recombinant proteins 1) to 7) above may not include His-Tag.

Referring to FIGS. 1 and 10, the Lin28 recombinant proteins of the present invention may be:

1) His-NLS-MTD₈₄-Lin28 (HNM₈₄Lin28), wherein His-NLS and a JO-84 MTD are fused to the N-terminus of Lin28;

2) His-NLS-Lin28-MTD₈₄ (HNLin28M₈₄), wherein a His-NLS is fused to the N-terminus and a JO-84 MTD is fused to the C-terminus of Lin28;

3) His-NLS-MTD₈₄-Lin28-MTD₈₄ (HNM₈₄Lin28M₈₄), wherein a His-NLS-JO-84 MTD is fused to the N-terminus and a JO-84 MTD is fused to the C-terminus of Lin28;

4) His-NLS-MTD₈₆-Lin28 (HNM₈₆Lin28), wherein a His-NLS and a JO-86 MTD are fused to the N-terminus of Lin28;

5) His-NLS-Lin28-MTD₈₆ (HNLin28M₈₆), wherein a His-NLS is fused to the N-terminus and a JO-86 MTD is fused to the C-terminus of Lin28;

6) His-NLS-MTD₈₆-Lin28-MTD₈₆ (HNM₈₆Lin28M₈₆), wherein a His-NLS-JO-86 MTD is fused to the N-terminus and a JO-86 MTD is fused to the C-terminus of Lin28; or

7) His-NLS-MTD₁₃₂-Lin28 (HNM₁₃₂Lin28), wherein a His-NLS and a JO-132 MTD are fused to the N-terminus of Lin28.

Optionally, the recombinant proteins 1) to 7) above may not include His-Tag.

As a control for the cell permeable Nanog recombinant proteins, His-NLS-Nanog (HNNanog), wherein only NLS and a histidine-tag are fused to Nanog without any MTDs, is prepared. This control protein has an amino acid sequence represented by SEQ ID NO: 34 and can be encoded by a polynucleotide having a nucleotide sequence represented by SEQ ID NO: 33.

As a control for the cell permeable Oct4 recombinant proteins, His-NLS-Oct4 (HNOct4), wherein only NLS and a histidine-tag are fused to Oct4 without any MTDs, is prepared. This control protein has an amino acid sequence represented by SEQ ID NO: 62 and can be encoded by a polynucleotide having a nucleotide sequence represented by SEQ ID NO: 61.

As a control for the cell permeable Sox2 recombinant proteins, His-NLS-Sox2 (HNSox2), wherein only NLS and a histidine-tag are fused to Sox2 without any MTDs, is prepared. This control protein has an amino acid sequence represented by SEQ ID NO: 90 and can be encoded by a polynucleotide having a nucleotide sequence represented by SEQ ID NO: 89.

As a control for the cell permeable Klf4 recombinant proteins, His-NLS-Klf4 (HNKlf4), wherein only NLS and a histidine-tag are fused to Klf4 without any MTDs, is prepared. This control protein has an amino acid sequence represented by SEQ ID NO: 118 and can be encoded by a polynucleotide having a nucleotide sequence represented by SEQ ID NO: 117.

As a control for the cell permeable cMyc recombinant proteins, His-NLS-cMyc (HNcMyc), wherein only NLS and a histidine-tag are fused to cMyc without any MTDs, is prepared. This control protein has an amino acid sequence represented by SEQ ID NO: 146 and can be encoded by a polynucleotide having a nucleotide sequence represented by SEQ ID NO: 145.

As a control for the cell permeable Lin28 recombinant proteins, His-NLS-Lin28 (HNLin28), wherein only NLS and a histidine-tag are fused to Lin28 without any MTDs, is prepared. This control protein has an amino acid sequence represented by SEQ ID NO: 174 and can be encoded by a polynucleotide having a nucleotide sequence represented by SEQ ID NO: 173.

Further, the present invention provides a recombinant expression vector comprising the polynucleotide encoding the cell permeable reprogramming transcription factor recombinant proteins described above, and a transformant prepared with this expression vector.

The term “recombinant expression vector” as used herein is a vector capable of expressing a target protein or a target RNA in a suitable host cell, and refers to a genetic construct comprising necessary regulatory elements operably linked to an insert gene such that the insert can be properly expressed.

As used herein, the term “operably linked” means that a nucleotide sequence encoding a target protein or a target RNA is functionally linked to regulatory elements in a manner that allows for the expression of the nucleotide sequence. For example, a promoter can be operably linked to a nucleotide sequence encoding a target protein or RNA to influence the expression of the nucleotide sequence. An operable linkage with an recombinant expression vector can be achieved by conventional gene recombinant techniques known in the art, while site-specific DNA cleavage and ligation are carried out by using conventional enzymes.

The expression vectors that can be used in the present invention may include, but are not limited to, plasmid vectors, cosmid vectors, bacteriophage vectors, viral vectors, etc. Suitable expression vectors may include a signal sequence or a leader sequence for membrane targeting or secretion, as well as regulatory sequences such as a promoter, an operator, an initiation codon, a termination codon, a polyadenylation signal, an enhancer, and the like. It can be prepared in various ways depending on the desired purpose. The promoter may be constitutive or inducible. Further, the expression vector may include one or more selection markers for screening a host cell containing the expression vector. In the case of a replicable expression vector, it may include a nucleotide sequence of the origin of replication.

The recombinant expression vector according to the present invention as constructed above may be, for example, pET28a(+)-HNM₈₆Nanog, pET28a(+)-HNM₈₆Oct4, pET28a(+)-HNM₈₆Sox2M₈₆, pET28a(+)-HNM₈₆Klf4M₈₆, pET28a(+)-HNM₈₆cMyc. In the recombinant expression vector encoding the cell permeable reprogramming transcription factor recombinant proteins according to the present invention, a polynucleotide encoding HM₈₆Nanog where a JO-86 MTD is fused to the N-terminus of Nanog; HM₈₆Oct4 where a JO-86 MTD is fused to the N-terminus of Oct4; HM₈₆Sox2M₈₆ where a JO-86 MTD is fused to the N-terminus and the C-terminus of Sox2; HM₈₆Klf4M₈₆ where a JO-86 MTD is fused to the N-terminus and the C-terminus of Klf4; or HM₈₆cMyc where a JO-86 MTD is fused to the N-terminus of cMyc is inserted in the NdeI restriction site within the multiple cloning sites (MCS) of a pET-28a(+) vector (Novagen, Germany).

In one embodiment of the present invention, the nucleotide sequence of the present invention is cloned into a pET-28a(+) vector (Novagen, Germany) having a His-Tag sequence so that the six cell permeable reprogramming transcription factor recombinant proteins have 6 histidine tags, which facilitate purification, expressed at the N-terminus.

The six cell permeable reprogramming transcription factor recombinant proteins expressed in the recombinant expression vector above have a structure where any one of JO-10 MTD, JO-52 MTD, JO-84 MTD, JO-86 MTD, JO-132 MTD, JO-145 MTD, JO-173 MTD, or JO-181 MTD is fused to either one or both termini of the recombinant proteins, and a His-Tag and/or NLS are linked to the N-terminus thereof.

The present invention further provides a transformant that is obtained by transforming a host cell with the recombinant expression vector above. Host cells suitable for the present invention may preferably be E. coli. E. coli may be transformed with the recombinant expression vector of the present invention, for example, pET28a(+)-HNM₈₆Nanog comprising a polynucleotide encoding HM₈₆Nanog in which a JO-86 MTD is fused to the N-terminus of the Nanog. The transformant thus obtained can be used to produce the cell permeable reprogramming transcription factor recombinant protein in large amounts. Any method of introducing a nucleic acid into a host cell may be used for the transformation. Any transformation techniques well known in the art may be used. Preferably, the methods may include, but are not limited to, microprojectile bombardment, electroporation, calcium phosphate (CaHPO₄) precipitation, calcium chloride (CaCl₂) precipitation, PEG-mediated fusion, microinjection, and liposome-mediated method.

In a preferred embodiment of the present invention, E. coli DH5α was transformed with the recombinant protein expression vectors prepared as described above, where NLS and JO-86 MTD are fused to the six reprogramming transcription factors. The transformant containing pET28a(+)-HNM₈₆Oct4 was deposited with the Korean Collection for Type Cultures (KCTC), Korea Research Institute of Bioscience and Biotechnology (KRIBB), on Feb. 17, 2010 as Deposit No. KCTC11640BP. E. coli DH5α was transformed with the recombinant expression vectors comprising pET28a(+)-HNM₁₀Nanog including JO-10 MTD, pET28a(+)-HNM₁₈₁Sox2 including JO-181 MTD, pET28a(+)-HNM₈₆cMyc including JO-86 MTD, pET28a(+)-HNM₁₇₃Klf4 including JO-173 MTD, and pET28a(+)-HNM₁₃₂Lin28 including JO-132 MTD to obtain transformants, respectively. These transformants were deposited with the Korean Collection for Type Cultures (KCTC), Korea Research Institute of Bioscience and Biotechnology (KRIBB), on Mar. 10, 2010 as Deposit Nos. KCTC11660BP, KCTC11659BP, KCTC11661BP, KCTC11662BP, and KCTC11663BP, respectively.

The present invention also provides a method of producing six kinds of cell permeable reprogramming transcription factor recombinant proteins comprising culturing the transformant.

The production method above is carried out by culturing the transformant in a suitable medium under suitable conditions so that a polynucleotide encoding the cell permeable reprogramming transcription factor recombinant protein of the present invention can be expressed. The method above is well known in the art and, for example, may be carried out by inoculating a transformant in a suitable medium for growing the transformant, performing a subculture, transferring the same to a main culture medium, culturing it under suitable conditions, for example, in the presence of isopropyl-β-D-thiogalactoside (IPTG), a gene expression inducer, thereby inducing the expression of the recombinant protein. After the culture is completed, it is possible to recover a substantially pure recombinant protein from the culture solution above. The term “substantially pure” means that the recombinant protein of the present invention and the polynucleotide encoding the same are essentially free of other proteins derived from the host cell.

The recombinant protein obtained above may be recovered by various isolation and purification methods known in the art. Conventionally, cell lysates are centrifuged to remove cell debris and impurities, and then subject to precipitation by, for example, salting out (ammonium sulfate precipitation or sodium phosphate precipitation) or solvent precipitation (protein-containing fragment precipitation using acetone, ethanol, etc.). Further, dialysis, electrophoresis, and various column chromatographies may be performed. With respect to chromatography, ion exchange chromatography, gel permeation chromatography, HPLC, reverse phase HPLC, affinity column chromatography, and ultrafiltration may be used alone or in combination (Maniatis et al., Molecular Cloning: A Laboratory Manual, Cold Spring Harbor Laboratory, Cold Spring Harbor, N.Y., 1982; Sambrook et al., Molecular Cloning: A Laboratory Manual, 2d Ed., Cold Spring Harbor Laboratory Press, 1989; Deutscher, M., Guide to Protein Purification Methods Enzymology vol. 182. Academic Press. Inc., San Diego, Calif., 1990).

Meanwhile, the recombinant protein expressed in the transformant transformed with the recombinant expression vector can be separated into a soluble fraction and an insoluble fraction according to the characteristics of the proteins during protein purification. If the majority of the expressed recombinant proteins are present in the soluble fraction, the recombinant protein can easily be isolated and purified according to the method described above. However, when the majority of the expressed recombinant proteins are present in the insoluble fraction, i.e., as inclusion bodies, the recombinant proteins can be solubilized using polypeptide denaturing agents, e.g., urea or surfactants, and then, centrifuged, followed by various treatments, such as dialysis, electrophoresis, and column chromatography for purification. Since there is a risk of losing the recombinant protein's activity due to structural modifications caused by solutions containing polypeptide denaturing agents, the process of purifying the recombinant protein from the insoluble fraction requires desalting and refolding steps. Namely, the desalting and refolding steps can be performed by dialysis and dilution with a solution that does not include a polypeptide denaturing agent or by centrifugation with a filter. Further, if the salt concentration of a solution used for the purification of a recombinant protein from a soluble fraction is high, such desalting and refolding steps may also be performed.

In one embodiment of the present invention, if it is confirmed that the cell permeable reprogramming transcription factor recombinant proteins of the present invention are present in the insoluble fraction as inclusion bodies, in order to purify the recombinant protein from the insoluble fraction, the insoluble fraction may be dissolved in a lysis buffer containing a non-ionic surfactant such as Triton X-100, subjected to ultrasonification, and then centrifuged to separate the precipitate. The resulting precipitate may be dissolved in a buffer containing a denaturing agent, such as urea, and centrifuged to separate the supernatant. The recombinant protein of the present invention, which is obtained by dissolving the insoluble fraction to the maximum extent with urea, was purified by means of a histidine-binding protein purification kit and subjected to dialysis, for example, by using an amicon filter for salt removal and protein refolding, thereby obtaining the purified recombinant protein of the present invention.

Further, the present invention provides a composition comprising the six cell permeable reprogramming transcription factor proteins described above. The composition according to the present invention is capable of establishing induced pluripotent stem cells when treated on somatic cells free of any reprogramming transcription factor.

This composition is capable of inducing pluripotency and self-renewal in a cell while maintaining the undifferentiated state of the cell by introducing a cell permeable reprogramming transcription factor into the nucleus of the cell with high efficiency. In addition, the composition can be usefully employed to induce reprogramming of somatic cells by activating genes specifically expressed in stem cells, thereby enabling establishment of induced pluripotent stem cells which are similar to embryonic stem cells.

Hereinafter, the embodiments of the present invention will be described in more detail with reference to the following examples. However, the examples are only provided for purposes of illustration and are not to be construed as limiting the scope of the invention.

### EXAMPLES

### Example 1

### Expression of Recombinant Proteins

In order to prepare gene constructs of the above-described recombinant proteins, polymerase chain reactions (PCRs) were carried out using a primer pair specifically designed for each construct and human Nanog, Oct4, Sox2, Klf4, c-Myc, and Lin28 cDNAs as a template.

The forward and reverse primers for amplifying HNNanog have nucleotide sequences represented by SEQ ID NOS: 187 and 188, respectively;

Those for amplifying HNM₈₄Nanog have nucleotide sequences represented by SEQ ID NOS: 189 and 188, respectively;

Those for amplifying HNNanogM₈₄ have nucleotide sequences represented by SEQ ID NOS: 187 and 190, respectively;

Those for amplifying HNM₈₄NanogM₈₄ have nucleotide sequences represented by SEQ ID NOS: 189 and 190, respectively;

Those for amplifying HNM₈₆Nanog have nucleotide sequences represented by SEQ ID NOS: 191 and 188, respectively;

Those for amplifying HNNanogM₈₆ have nucleotide sequences represented by SEQ ID NOS: 187 and 192, respectively;

Those for amplifying HNM₈₆NanogM₈₆ have nucleotide sequences represented by SEQ ID NOS: 191 and 192, respectively;

Those for amplifying HNOct4 have nucleotide sequences represented by SEQ ID NOS: 193 and 194, respectively;

Those for amplifying HN M₈₄Oct4 have nucleotide sequences represented by SEQ ID NOS: 195 and 194, respectively;

Those for amplifying HNOct4M₈₄ have nucleotide sequences represented by SEQ ID NOS: 193 and 190, respectively;

Those for amplifying HNM₈₄Oct4M₈₄ have nucleotide sequences represented by SEQ ID NOS: 195 and 196, respectively;

Those for amplifying HNM₈₆Oct4 have nucleotide sequences represented by SEQ ID NOS: 197 and 194, respectively;

Those for amplifying HNOct4M₈₆ have nucleotide sequences represented by SEQ ID NOS: 193 and 198, respectively;

Those for amplifying HNM₈₆Oct4M₈₆ have nucleotide sequences represented by SEQ ID NOS: 197 and 198, respectively;

Those for amplifying HNSox2 have nucleotide sequences represented by SEQ ID NOS: 199 and 200, respectively;

Those for amplifying HNM₈₄Sox2 have nucleotide sequences represented by SEQ ID NOS: 201 and 200, respectively;

Those for amplifying HNSox2M₈₄ have nucleotide sequences represented by SEQ ID NOS: 199 and 202, respectively;

Those for amplifying HNM₈₄Sox2M₈₄ have nucleotide sequences represented by SEQ ID NOS: 201 and 202, respectively;

Those for amplifying HNM₈₆Sox2 have nucleotide sequences represented by SEQ ID NOS: 203 and 200, respectively;

Those for amplifying HNSox2M₈₆ have nucleotide sequences represented by SEQ ID NOS: 199 and 204, respectively;

Those for amplifying HNM₈₆Sox2M₈₆ have nucleotide sequences represented by SEQ ID NOS: 203 and 204, respectively;

Those for amplifying HNKlf4 have nucleotide sequences represented by SEQ ID NOS: 205 and 206, respectively;

Those for amplifying HNM₈₄Klf4 have nucleotide sequences represented by SEQ ID NOS: 207 and 206, respectively;

Those for amplifying HNKlf4M₈₄ have nucleotide sequences represented by SEQ ID NOS: 205 and 208, respectively;

Those for amplifying HNM₈₄Klf4M₈₄ have nucleotide sequences represented by SEQ ID NOS: 207 and 208, respectively;

Those for amplifying HNM₈₆Klf4 have nucleotide sequences represented by SEQ ID NOS: 209 and 206, respectively;

Those for amplifying HNKlf4M₈₆ have nucleotide sequences represented by SEQ ID NOS: 205 and 210, respectively;

Those for amplifying HNM₈₆Klf4M₈₆ have nucleotide sequences represented by SEQ ID NOS: 209 and 210, respectively;

Those for amplifying HNcMyc have nucleotide sequences represented by SEQ ID NOS: 211 and 212, respectively;

Those for amplifying HNM₈₄cMyc have nucleotide sequences represented by SEQ ID NOS: 213 and 212, respectively;

Those for amplifying HNcMycM₈₄ have nucleotide sequences represented by SEQ ID NOS: 211 and 214, respectively;

Those for amplifying HNM₈₄cMycM₈₄ have nucleotide sequences represented by SEQ ID NOS: 213 and 214, respectively;

Those for amplifying HNM₈₆cMyc have nucleotide sequences represented by SEQ ID NOS: 215 and 212, respectively;

Those for amplifying HNcMycM₈₆ have nucleotide sequences represented by SEQ ID NOS: 211 and 216, respectively;

Those for amplifying HNM₈₆cMycM₈₆ have nucleotide sequences represented by SEQ ID NOS: 215 and 216, respectively;

Those for amplifying HNLin28 have nucleotide sequences represented by SEQ ID NOS: 217 and 218, respectively;

Those for amplifying HNM₈₄Lin28 have nucleotide sequences represented by SEQ ID NOS: 219 and 218, respectively;

Those for amplifying HNLin28M₈₄ have nucleotide sequences represented by SEQ ID NOS: 217 and 220, respectively;

Those for amplifying HNM₈₄Lin28M₈₄ have nucleotide sequences represented by SEQ ID NOS: 219 and 220, respectively;

Those for amplifying HNM₈₆Lin28 have nucleotide sequences represented by SEQ ID NOS: 221 and 218, respectively;

Those for amplifying HNLin28M₈₆ have nucleotide sequences represented by SEQ ID NOS: 217 and 222, respectively; and

Those for amplifying HNM₈₆Lin28M₈₆ have nucleotide sequences represented by SEQ ID NOS: 221 and 222, respectively.

The PCRs above were carried out in a 100 μl final volume reaction mixture containing 100 ng of human Nanog, Oct4, Sox2, Klf4, c-Myc, or Lin28 cDNAs as a template, 0.2 mM (final concentration) of a dNTP mixture (dGTP, dATP, dTTP, and dCTP, each at 2 mM), 0.5 μM of primers, 10 μl of a 10× Taq buffer, and 0.5 μl of Taq polymerase (Takara, Japan). The PCR reactions were performed 30 cycles at 95° C. for 45 seconds, 67° C. for 45 seconds, and 72° C. for 45 seconds after the initial denaturation at 95° C. for 2 minutes, followed by the final amplification at 72° C. for 5 minutes. After the reaction was completed, the amplified products were confirmed by electrophoresis on a 0.8% agarose gel.

After recovering the amplified recombinant fragments from the agarose gel, each recombinant fragment was extracted and purified using a commonly used kit (QIAquick Gel extraction kit, Qiagen, USA). The extracted fragments were inserted into a pGEM-T Easy vector (Promega, USA) and E. coli DH5α competent cells were transformed with the pGEM-T Easy vector in which an MTD-fused Nanog, Oct4, Sox2, Klf4,c-Myc, or Lin28 recombinant protein gene fragment was subcloned. The cells were plated onto an LB medium supplemented with 50 μg/ml ampicillin and cultured at 37° C. overnight. The transformed E. coli was screened and inoculated in the LB medium again to obtain pGEM-T Easy vectors in which a Nanog, Oct4, Sox2, Klf4,c-Myc, or Lin28 recombinant protein gene was inserted. As a result, a large amount of vectors were obtained.

The recombinant fragment-inserted pGEM-T Easy vectors were treated by an NdeI restriction enzyme (Enzynomics, Korea) to isolate the recombinant fragments. The isolated recombinant fragments were subjected to 0.8% agarose gel electrophoresis. As a result, the successful subcloning of each recombinant fragment into the vector was confirmed.

The pGEM-T Easy vectors in which a Nanog, Oct4, Sox2, Klf4, c-Myc, or Lin28 recombinant protein gene was inserted were digested with NdeI at 37° C. for 2 hours to obtain the recombinant fragment for each vector. After recovering the recombinant fragments from the agarose gel, each recombinant fragment was extracted and purified using a commonly used kit (QIAquick Gel extraction kit, Qiagen, USA) Meanwhile, an expression vector comprising a His-Tag and a T7 promoter, pET-28a(+) (Novagen, USA) was digested with NdeI under the same conditions above. Each of the recombinant fragments obtained above was admixed with the digested pET-28a(+) vector. With the addition of a T4 DNA ligase (Takara, Japan), the mixture was subjected to ligation at 16° C. for 12 hours. E. coli DH5α supercompetent cells were transformed to obtain the recombinant protein expression vectors.

The recombinant fragment-inserted pET-28a(+) vectors were treated by an NdeI restriction enzyme to isolate the recombinant fragments and the isolated recombinant fragments were subjected to 0.8% agarose gel electrophoresis. As a result, the successful subcloning of each recombinant fragment into the vector was confirmed.

The recombinant protein expression vectors thus obtained were designated as pET28a(+)-HNNanog, pET28a(+)-HNM₈₄Nanog, pET28a(+)-HNNanogM₈₄, pET28a(+)-HNM₈₄NanogM₈₄, pET28a(+)-HNM₈₆Nanog, pET28a(+)-HNNanogM₈₆, pET28a(+)-HNM₈₆NanogM₈₆, pET28a(+)-HNOct4, pET28a(+)-HNM₈₄Oct4, pET28a(+)-HNOct4M₈₄, pET28a(+)-HNM₈₄Oct4M₈₄, pET28a(+)-HNM₈₆Oct4, pET28a(+)-HNOct4M₈₆, pET28a(+)-HNM₈₆Oct4M₈₆, pET28a(+)-HNSox2, pET28a(+)-HNM₈₄Sox2, pET28a(+)-HNSox2M₈₄, pET28a(+)-HNM₈₄Sox2M₈₄, pET28a(+)-HNM₈₆Sox2, pET28a(+)-HNSox2M₈₆, pET28a(+)-HNM₈₆Sox2M₈₆, pET28a(+)-HNKlf4, pET28a(+)-HNM₈₄Klf4, pET28a(+)-HNKlf4M₈₄, pET28a(+)-HNM₈₄Klf4M₈₄, pET28a(+)-HNM₈₆Klf4, pET28a(+)-HNKlf4M₈₆, pET28a(+)-HNM₈₆Klf4M₈₆, pET28a(+)-HNcMyc, pET28a(+)-HNM₈₄cMyc, pET28a(+)-HNcMycM₈₄, pET28a(+)-HNM₈₄cMycM₈₄, pET28a(+)-HNM₈₆cMyc, pET28a(+)-HNcMycM₈₆, pET28a(+)-HNM₈₆cMycM₈₆, pET28a(+)-HNLin28, pET28a(+)-HNM₈₄Lin28, pET28a(+)-HNLin28M₈₄, pET28a(+)-HNM₈₄Lin28M₈₄, pET28a(+)-HNM₈₆Lin28, pET28a(+)-HN Lin28M₈₆, and pET28a(+)-HNM₈₆Lin28M₈₆, respectively.

E. coli BL21 CodonPlus (DE3) was transformed with each of the following recombinant expression vectors, i.e., pET28a(+)-HNNanog, pET28a(+)-HNM₈₄Nanog, pET28a(+)-HNNanogM₈₄, pET28a(+)-HNM₈₄NanogM₈₄, pET28a(+)-HNM₈₆Nanog, pET28a(+)-HNNanogM₈₆, pET28a(+)-HNM₈₆NanogM₈₆, pET28a(+)-HNM₁₀Nanog, pET28a(+)-HNOct4, pET28a(+)-HNM₈₄Oct4, pET28a(+)-HNOct4M₈₄, pET28a(+)-HNM₈₄Oct4M₈₄, pET28a(+)-HNM₈₆Oct4, pET28a(+)-HNOct4M₈₆, pET28a(+)-HNM₈₆Oct4M₈₆, pET28a(+)-HNM₅₂Oct4, pET28a(+)-HNSox2, pET28a(+)-HNM₈₄Sox2, pET28a(+)-HNSox2M₈₄, pET28a(+)-HNM₈₄Sox2M₈₄, pET28a(+)-HNM₈₆Sox2, pET28a(+)-HNSox2M₈₆, pET28a(+)-HNM₈₆Sox2M₈₆, pET28a(+)-HNM₁₈₁Sox2, pET28a(+)-HNKlf4, pET28a(+)-HNM₈₄Klf4, pET28a(+)-HNKlf4M₈₄, pET28a(+)-HNM₈₄Klf4M₈₄, pET28a(+)-HNM₈₆Klf4, pET28a(+)-HNKlf4M₈₆, pET28a(+)-HNM₈₆Klf4M₈₆, pET28a(+)-HNM₁₇₃Klf4, pET28a(+)-HNcMyc, pET28a(+)-HNM₈₄cMyc, pET28a(+)-HNcMycM₈₄, pET28a(+)-HNM₈₄cMycM₈₄, pET28a(+)-HNM₈₆cMyc, pET28a(+)-HNcMycM₈₆, pET28a(+)-HNM₈₆cMycM₈₆, pET28a(+)-HNM₁₄₅cMyc, pET28a(+)-HNLin28, pET28a(+)-HNM₈₄Lin28, pET28a(+)-HNLin28M₈₄, pET28a(+)-HNM₈₄Lin28M₈₄, pET28a(+)-HNM₈₆Lin28, pET28a(+)-HN Lin28M₈₆, pET28a(+)-HNM₈₆Lin28M₈₆, and pET28a(+)-HNM₁₃₂Lin28, by a heat shock method. The transformants were cultured in an LB medium containing 50 μg/ml of kanamycin. Thereafter, E. coli transformed with DNA encoding the recombinant protein was inoculated in 25 ml of an LB medium and cultured at 37° C. overnight, and then inoculated again in 1 L of an LB medium and cultured at 37° C. until the optical density OD₆₀₀ reached 0.6 to 0.7. To the culture was added 0.65 mM isopropyl-β-D-thiogalactoside (IPTG) as a protein expression inducer, followed by incubation at 37° C. for an additional 3 hours. This culture was centrifuged at 4° C. at a speed of 4,000×g for 20 minutes to remove the supernatant and bacterial cells were harvested. The harvested bacterial cells were suspended in a lysis buffer (100 mM NaH₂PO₄, 10 mM Tris-HCl, 8 M urea, pH 8.0) and the suspension was subject to sonication to disrupt the cells. The cell lysates were centrifuged at a speed of 14,000×g for 15 minutes to separate the insoluble fraction from the soluble fraction. The soluble and insoluble fractions thus obtained were loaded on an SDS-PAGE gel separately to analyze the protein expression profile and the degree of expression.

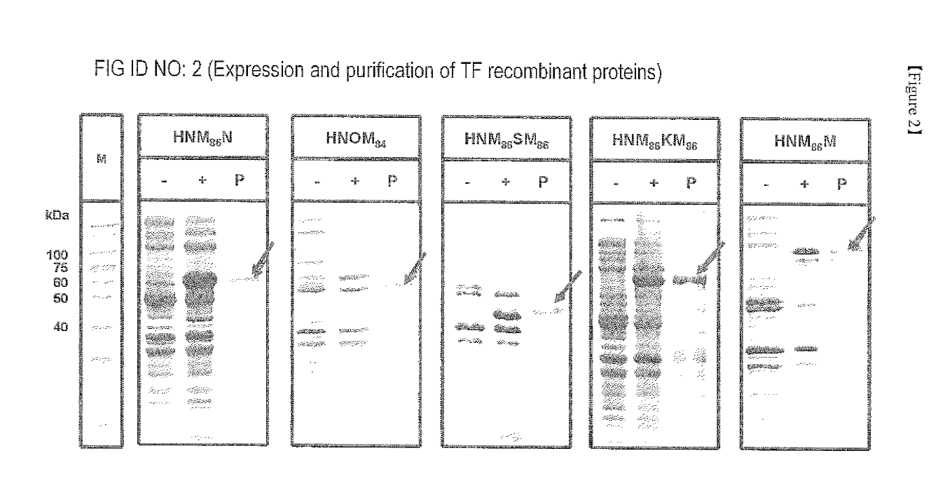

As shown in FIG. 2, the cell permeable reprogramming transcription factor recombinant proteins according to the present invention (about 34 kDa of Nanog, about 40 kDa of Oct4, about 35 kDa of Sox2, about 52 kDa of Klf4, and about 50 kDa of cMyc) are mostly present in the insoluble fraction as inclusion bodies. Further, it was found that expression of the protein significantly increased in the culture solution with IPTG (+) compared to that without IPTG (−).

### Example 2

### Purification of the Recombinant Proteins

Since the cell permeable reprogramming transcription factor recombinant proteins according to the present invention are present in the insoluble fraction as inclusion bodies, 8 M urea was used as a strong denaturing agent to separate these proteins from the insoluble fraction.

First, the BL21 CodonPlus (DE3) strains transformed with each of the expression vectors of the present invention were cultured in 1 L of an LB medium as described in Example 1 above. Each culture solution was centrifuged to harvest the bacterial cells. The obtained bacterial cells were gently suspended in 20 ml of a lysis buffer (100 mM NaH₂PO₄, 10 mM Tris-HCl, 8 M urea, pH 8.0) carefully so as to avoid forming bubbles, and homogenized at a low temperature using an ultrasonic homogenizer equipped with a microtip to destruct the cells. Here, the power was set at 25% the maximum power, while a 45 second sonication followed by a 10 second pause was repeated for 7 minutes. The sufficiently lysed inclusion bodies were centrifuged at 4° C. at a speed of 4,000×g for 20 minutes to remove the cell precipitate and recover the supernatant. The recovered supernatant was loaded onto an Ni-NTA agarose resin where nickel (Ni) was added on nitrilotriacetic acid agarose. The Ni-NTA agarose was used after equilibration by washing with a lysis buffer prior to use. The supernatant was allowed to absorb onto the resin while slowly stirring using a rotary shaker for at least 8 hours at 4° C. The resin absorbed with the inclusion bodies containing the recombinant protein was centrifuged at 4° C. at a speed of 1,000 rpm for 5 minutes to remove the reaction solution and then washed with a washing buffer (100 mM NaH₂PO₄, 10 mM Tris-HCl, 8 M urea, pH 6.3) five times to remove the non-specifically absorbed materials. Onto the washed resin was loaded an elution buffer (100 mM HaH₂PO₄, 10 mM Tris-HCl, 8 M urea, pH 4.5) in a volume that is twice the resin volume under acidic conditions of pH 4.0, followed by stirring in a shaker for 2 hours or at least 8 hours to elute the protein. In order to analyze the purity of the eluted protein, electrophoresis was carried out on a 12% SDS-PAGE gel, and subsequently, the gel was stained with Coomassie Brilliant Blue R with gentle shaking, and de-stained with a de-staining solution until the band of the target protein could be seen clearly.

As a result, as shown in FIG. 2, all of the cell permeable reprogramming transcription factor recombinant proteins fused to a JO-84 MTD or a JO-86 MTD were detected as separate bands in comparison with the band of the marker protein. It was confirmed from the results above that the cell permeable reprogramming transcription factor recombinant proteins of the present invention were purified from the insoluble fraction.

Since the recombinant proteins of the present invention purified from the insoluble fraction above were denatured with 8 M urea, a strong denaturing agent, a refolding process was carried out to convert them to an active form as described below:

First, the purified recombinant proteins were subjected to dialysis using a refolding buffer (0.55 M Guanidine HCl, 0.88 M L-arginine, 50 mM Tris-HCl, 150 mM NaCl, 1 mM EDTA, 100 mM NDSB, 2 mM glutathione oxidized, and 1 mM glutathione reduced) at 4° C. for at least 72 hours to remove the denaturing agent. By doing so, the recombinant proteins were reactivated, namely, refolded. The refolding buffer in the container was changed every 24 hours. Thereafter, the activated recombinant proteins were dialyzed in a dialysis tubing (Snakeskin pleated, PIERCE) using a DMEM (Dulbecco's Modified Eagle Medium) containing 1% penicillin/streptomycin at 4° C. for 9 hours while stirring. The medium in the tubing was replaced every 3 hours. The cell permeable reprogramming transcription factor recombinant proteins thus refolded were used in the following experiments.

### Example 3

### Induction of the Reprogrammed Stem Cells and Alkaline Phosphatase Staining (AP Staining)

Human dermal fibroblast (HDF) cells were cultured to carry out induction of the reprogrammed stem cells. For an initial 7 days, the cells were cultured at 37° C. in a humidified atmosphere of 5% CO₂ in an HDF medium (M106 (Cascade Biologic™)+LSGS (Cascade Biologic™)), and for a subsequent 14 days, the HDF medium was exchanged with a human embryonic stem cell medium (DMEM/F12 (HyClone®) supplemented with 20% KSR (GIBCO®), 2 mM L-glutamin (GIBCO®), 2 mM MEM Non Essential Amino Acid (GIBCO®), 0.1 mM β-mercaptoethanol (GIBCO®), and 0.1% penicillin/streptomycin (GIBCO®)). The cells were treated with the five cell permeable reprogramming transcription factor recombinant proteins at a concentration of 1 μM, and the cells treated with the MTD-free reprogramming transcription factor recombinant proteins at the same concentration were used as a control group. The treatment was conducted for 16 days, while the media and proteins were changed everyday. 16 days after the treatment, the total number of colonies formed was 23 and these colonies were used in examining the formation of reprogrammed stem cells.

In order to confirm that the colonies were reprogrammed stem cells, alkaline phosphatase (AP) staining was carried out (Sigma). Undifferentiated embryonic stem cells are characterized by a high expression level of alkaline phosphatase. Alkaline phosphatase (AP) is not only found in many organs in the body but also in the cytoplasm of an embryonic stem cell. The presence of AP can be detected by being stained in red. A capsule of Fast Blue RR salt in the alkaline phosphatase staining kit was dissolved in water to prepare a diazonium salt solution.

Naphthol AS-MX Phosphate Alkaline Solution was added to the diazonium salt solution to prepare an alkaline staining mixture. The cells in the 6-well plate were fixed with a fixative solution for about 30 seconds and rinsed gently with water for 45 seconds. With addition of the alkaline staining mixture, the cells were incubated at room temperature for 30 minutes while making sure the samples were not exposed to sunlight. Upon completion of the incubation, the cells were washed with water. Counterstaining was performed on these cells with a Mayer's Hematoxylin solution for 10 minutes and then rinsed with water for 2 minutes. Thereafter, staining was evaluated microscopically after covering the cells with a mounting solution.

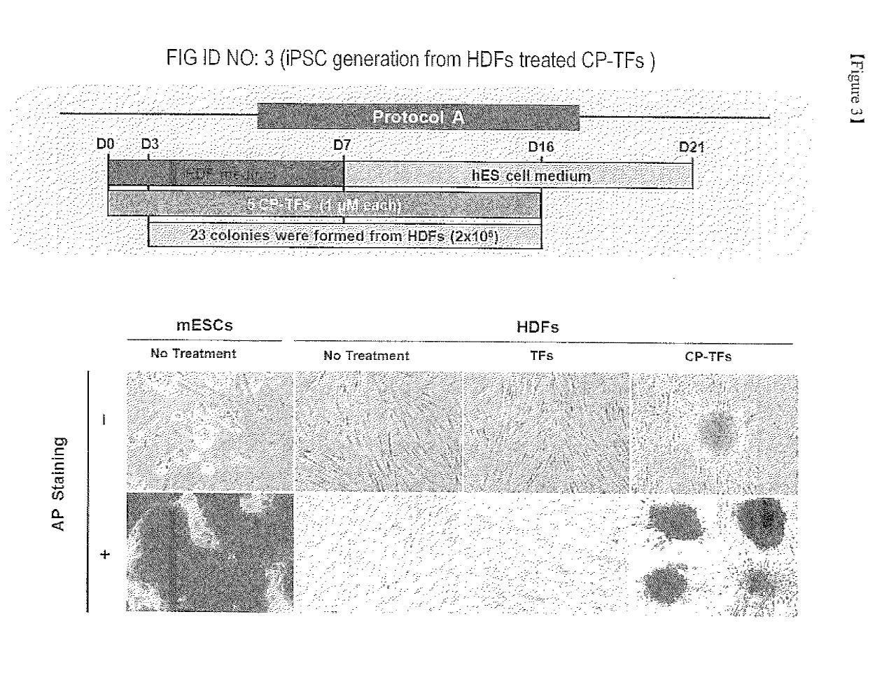

As a result, as shown in FIG. 3, the undifferentiated state of the mouse embryonic stem cells was confirmed by AP staining, which is a characteristic of embryonic stem cells. The colonies, which had been formed by treatment with the cell permeable reprogramming transcription factor recombinant proteins for 16 days, also were found to be stained in red by AP staining, which is a marker indicative of cell undifferentiation.

### Example 4

### Immunocytochemistry

Colonies of the induced pluripotent stem cells were obtained by treating human dermal fibroblast cells (HDF) with the cell permeable recombinant proteins described in Example 3. The colonies were scrapped by using a capillary glass tube which had been bent into a ring shape by heating with an alcohol lamp. The successful removal of the colonies was confirmed under a stereomicroscope. The colonies were loaded for adhesion onto the 8-well chamber slides (chamber slide, NUNC) on which human dermal fibroblast cells were plated. After 1 day of stabilization, the medium was removed from the chamber slide and rinsed twice with PBS (phosphate buffered saline; WelGENE). The colonies were fixed in 2% paraformaldehyde (JUNSEI) for 20 minutes, washed twice with PBS again, and treated with 0.1% Triton X-100 for 5 minutes to make the cell membranes permeable. After rinsing with PBS twice, the cells were treated with 2% BSA (bovine serum albumin; Sigma)/PBS at room temperature for 1 hour to block non-specific protein binding. Thereafter, the colonies were treated with primary antibodies (diluted 1:1000 in 2% BSA/PBS) overnight in a refrigerator at 4° C. The primary antibodies used against Nanog and Oct4 were anti-Nanog antibody (abcam) and anti-Oct4 antibody (abcam), respectively. The colonies were washed twice with PBS upon completion of the reaction and then treated with secondary antibodies (diluted 1:500 with 2% BSA/PBS) at room temperature for 1 hour. The secondary Nanog and Oct4 antibodies used were Alexa Fluor® 488 goat anti-rabbit IgG (Alexa) and Alexa Fluor® 546 rabbit anti-goat IgG (Alexa), respectively. After rinsing the colonies with PBS three times, the nuclei were stained with 300 nM DAPI (4,6-diamidino-2-phenylindole, Sigma) at room temperature for 5 minutes in darkness. The colonies were washed with PBS three times. A mounting medium (VECTOR) was mounted thereon, and after 15 minutes, a laser scanning confocal microscopy with a Nomarski filter was used for observation. The original shape of cells and fluorescence were examined for Nanog excited at 488 nm and for Oct4 excited at 546 nm.

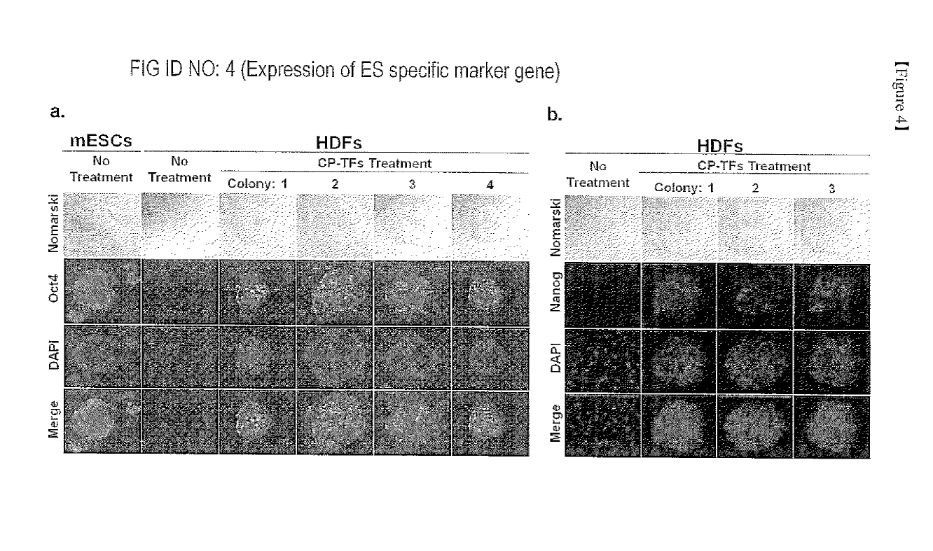

As a result, as shown in FIG. 4, it was found that the pluripotent stem cells induced by the cell permeable reprogramming transcription factor recombinant proteins according to the present invention expressed Nanog and Oct 4, which is regarded to be a characteristic of stem cells.

### Example 5

### Expression of a Stem Cell Specific Marker on the Surface of an Induced Pluripotent Stem Cell Established by a Reprogramming Transcription Factor

The colonies that were confirmed to have an undifferentiated state by AP staining in Example 4 were treated with trypsin/EDTA (T/E, Invitrogen) at 37° C. for 3 minutes to disaggregate into single cells. The cells were washed with PBS supplemented with 0.1% BSA twice and treated with Fc Blocker CD16/32 (Fc Blocker, BD Pharmingen) for 10 minutes to block non-specific binding of the Fc region to the FcR on the cell surface. The treated cells were washed with PBS supplemented with 0.1% BSA twice, and stained on ice with TRA-1-60 (Tumor Rejection Antigen, BD bioscience), at a concentration of 1 μg/1×10⁶ for 20 minutes, FTTC (fluorescein-5-isothiocyanate)-labeled SSEA-4 (Stage Specific Embryonic Antigen-4) antibody (BD bioscience), and PE (Phycoerythrin)-labeled SSEA-3 antibody (Stage Specific Embryonic Antigen-3) antibody (BD bioscience). Then, the cells were washed with PBS (Welgene) supplemented with 0.1% BSA twice. The prepared cells were analyzed with Calibur flow cytometry (Beckton-Dickinson, CA) using the CellQuest Pro cytometric analysis software (CellQuest Pro, BD).

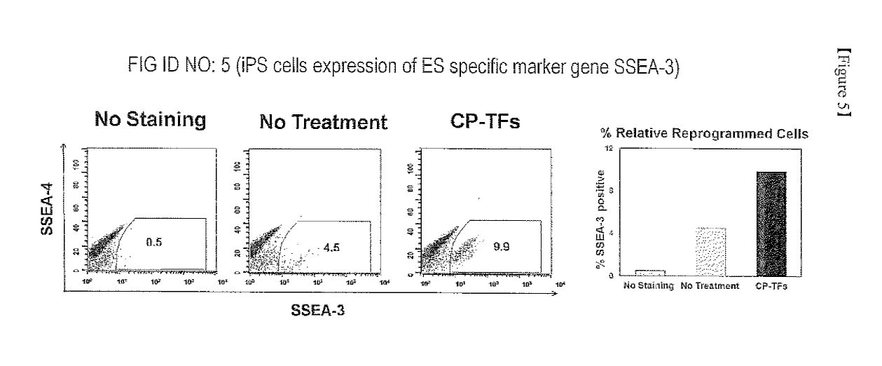

As a result, as shown in FIG. 5, it was found that the expression of the stem cell specific surface marker of the colonies induced by the cell permeable reprogramming transcription factor recombinant proteins according to the present invention showed higher fluorescent intensity when compared to the control group.

In FIG. 5, no staining indicated the group to which the reprogramming transcription factors were not treated, and only 0.5% of the cells expressed SSEA-3. 4.5% of the cells expressed SSEA-3 in the MTD-free control group, whereas in the colonies formed by treatment with the cell permeable reprogramming transcription factors, 9.9% of the cells expressed SSEA-3.

As a result, as shown in FIG. 5, it was confirmed that TRA-1-60, the stem cell specific surface marker of the colony induced by the cell permeable reprogramming transcription factor recombinant proteins according to the present invention, showed a higher level of expression when compared to the control group by at least 5%.

### Example 6

### Reverse Transcriptase Polymerase Chain Reaction (RT-PCR)

RT-PCR was performed to confirm whether the induced reprogrammed stem cells described above showed an increased expression level of stem cell marker genes. RNAs were extracted from cells using a Trizol (iNTRON), and 5 μg of RNA was subjected to reverse transcription (Roche). A PCR was performed to examine the difference in expression levels of stem cell marker genes between the cDNAs from the group treated with the cell permeable reprogramming transcription factor recombinant proteins to induce reprogramming and those from the control group not treated with the recombinant proteins. A total of 14 genes were used as marker genes: Oct4, Nanog, LEFTB, Klf4, cMyc, EBAF, UTF1, CD9, ESG1, IFITM1, GAL, BRIX, and REX1. Further, for the control group, GAPDH was used as it is universally expressed in all types of cells. The PCR reaction was repeated 30-35 cycles at 95° C. for 1 minute, 55° C. for 1 minute, and 72° C. for 1 minute. Electrophoresis was carried out with the PCR product on an agarose gel.

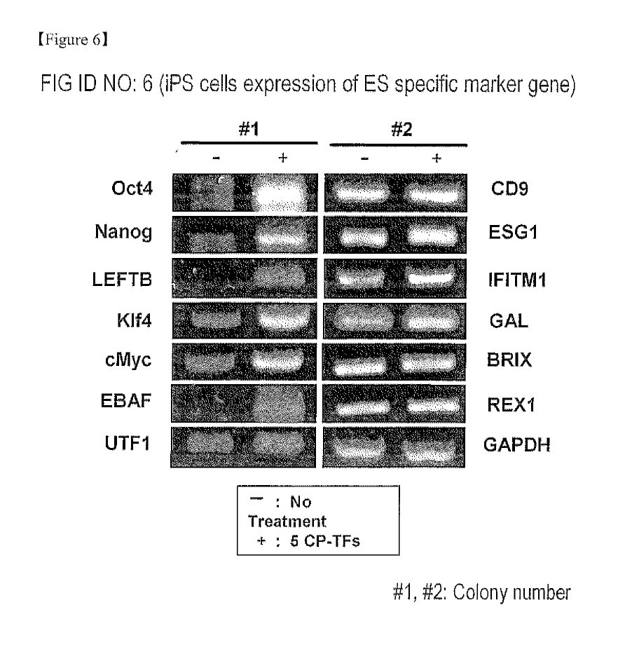

As shown in FIG. 6, it was found that the group treated with the cell permeable reprogramming transcription factor recombinant proteins exhibited a higher expression level of the genes specifically expressed in stem cells when compared to the control group.

### Example 7

### Induction of the Reprogrammed Stem Cells

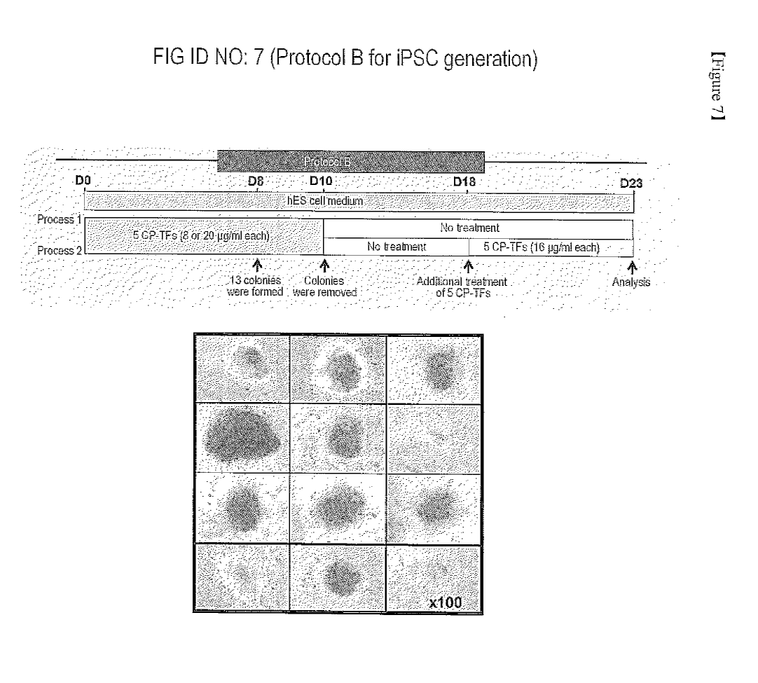

Human dermal fibroblast (HDF) cells were cultured to carry out induction of the reprogrammed stem cells. As shown in FIG. 7, the cells were cultured for a period of 23 days in a human stem cell medium (DMEM/F12 (HyClone®), 20% KSR (GIBCO®), 2 mM L-glutamin (GIBCO®), 2 mM MEM Non Essential Amino Acid (GIBCO®), 0.1 mM β-mercaptoethanol (GIBCO®), and 0.1% penicillin/streptomycin (GIBCO®)). The cells were treated with the five cell permeable reprogramming transcription factor recombinant proteins at a concentration of 8 μg/ml or 20 μg/ml. A control group was treated with MTD-free reprogramming transcription factor recombinant proteins at the same concentration. The treatment was carried out for 10 days while the medium and the proteins were replaced daily. On the 8^{th} day after the protein treatment, a total of 13 colonies were formed. The colonies were then scrapped 10 days after the protein treatment using a capillary glass tube which had been bent into a ring shape by heating with an alcohol lamp. The successful removal of the colonies was confirmed under a stereomicroscope. Thereafter, the colonies were divided into a group with no further treatment with protein and a group to which 16 μg/ml of protein was treated for another 5 days from 8 days after the removal. Morphology of the colonies is illustrated in FIG. 7. The collected colonies were used in examining the formation of reprogrammed stem cells below.

### Example 8

### Immunocytochemistry

Colonies of the induced pluripotent stem cell colony obtained by treating human dermal fibroblast cells (HDF) with the cell permeable recombinant proteins described in Example 7 were loaded for adhesion onto the 8-well chamber slides (NUNC) on which mouse dermal fibroblast cells were plated. After 1 day of stabilization, the medium was removed from the chamber slide and the slides were rinsed twice with PBS (WelGENE). The colonies were fixed in 2% paraformaldehyde (JUNSEI) for 20 minutes, washed twice with PBS again, and treated with 0.1% Triton X-100 for 5 minutes to make the cell membranes permeable. After rinsing with PBS twice, the colonies were treated with 2% BSA (Sigma)/PBS at room temperature for 1 hour to block non-specific protein binding.

Thereafter, the colonies were treated with primary antibodies (diluted 1:1,000 in 2% BSA/PBS) overnight in a refrigerator at 4° C. The primary antibodies used were anti-Nanog antibody (abcam) and anti-Oct4 antibody (abcam). The colony was washed twice with PBS upon completion of the reaction and then treated with secondary antibodies (diluted 1:500 with 2% BSA/PBS) at room temperature for 1 hour. The secondary antibodies used against Nanog and Oct4 were Alexa Fluor® 488 goat anti-rabbit IgG (Alexa) and Alexa Fluor® 546 rabbit anti-goat IgG (Alexa), respectively. After rinsing with PBS three times, the nuclei were stained with 300 nM DAPI (Sigma) at room temperature for 5 minutes in darkness. The colonies were washed with PBS three times. A mounting medium (VECTOR) was mounted on the slides, and after 15 minutes, a laser scanning confocal microscopy with a Nomarski filter was used for observation. The original shape of cells and fluorescence were examined for Nanog excited at 488 nm and for Oct4 excited at 546 nm.

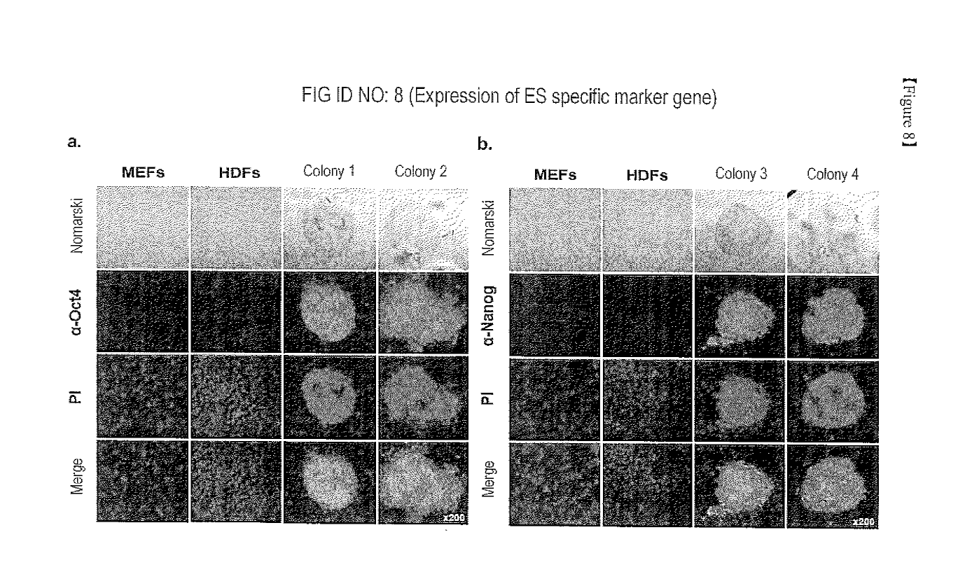

As a result, as shown in FIG. 8, it was found that the pluripotent stem cells induced by the cell permeable reprogramming transcription factor recombinant proteins according to the present invention expressed Nanog and Oct4, which is regarded to be a characteristic of stem cells.

### Example 9

### Expression of a Stem Cell Specific Marker on the Surface of an Induced Pluripotent Stem Cell Established by a Reprogramming Transcription Factor

The colonies that were confirmed to have an undifferentiated state by AP staining in the Example above were treated with trypsin/EDTA (T/E, Invitrogen) at 37° C. for 3 minutes to disaggregate into single cells. The cells were washed with PBS supplemented with 0.1% BSA twice and treated with Fc Blocker CD16/32 (Fc Blocker, BD Pharmingen) for 10 minutes to block non-specific binding of the Fc region to the FcR on the cell surface. The treated cells were washed with PBS (phosphate buffered saline) supplemented with 0.1% BSA twice, and stained on ice with TRA-1-60 (Tumor Rejection Antigen, BD bioscience), at a concentration of 1 μg/1×10⁶ for 20 minutes, FTTC (fluorescein-5-isothiocyanate)-labeled SSEA-4 (Stage Specific Embryonic Antigen-4) antibody (BD bioscience), and PE (Phycoerythrin)-labeled SSEA-3 antibody (Stage Specific Embryonic Antigen-3) antibody (BD bioscience). Then, the cells were washed with PBS (Welgene) supplemented with 0.1% BSA twice. The prepared cells were analyzed with Calibur flow cytometry (Beckton-Dickinson, CA) using the CellQuest Pro cytometric analysis software (CellQuest Pro, BD).

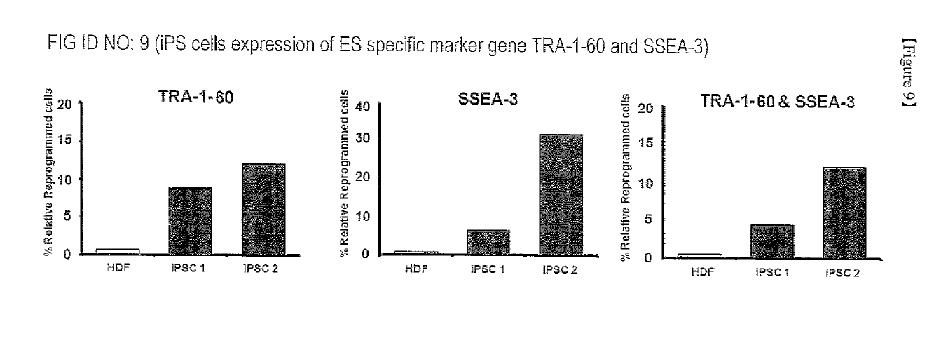

As a result, as shown in FIG. 9, it was found that the expression of the stem cell specific surface marker in the colonies induced by the cell permeable reprogramming transcription factor recombinant proteins according to the present invention showed higher fluorescent intensity when compared to the control group.

In FIG. 9, HDF represents the control group not treated with the reprogramming transcription factors, and less than about 1% of the cells expressed SSEA-3. In the case of the colonies formed by treatment with the cell-permeable reprogramming transcription factors, about 5% and about 30% of the cells expressed SSEA-3 for each group.

As a result, as shown in FIG. 9, it was found that the expression level of TRA-160, which is a stem cell specific surface marker, was higher in the colonies induced by the cell permeable reprogramming transcription factor recombinant proteins according to the present invention when compared to the control group by about at least 10-12%.

### Example 10

### Expression of Codon Optimized Recombinant Proteins

E. coli BL21 CodonPlus (DE3) was transformed with any of the following expression vectors, i.e., pET28a(+)-HNM₁₀Nanog, pET28a(+)-HNM₅₂Oct4, pET28a(+)-HNM₁₈₁Sox2, pET28a(+)-HNM₁₇₃Klf4, pET28a(+)-HNM₁₄₅cMyc, and pET28a(+)-HNM₁₃₂Lin28, in which a codon-optimized recombinant protein synthesized by Genscript was subcloned by a heat shock method, followed by incubation in an LB medium containing 50 μg/ml of kanamycin. Thereafter, the transformant was inoculated in 25 ml of an LB medium and cultured at 37° C. overnight. Then, the culture was inoculated again in 1 l of an LB medium and incubated at 37° C. until the optical density OD₆₀₀ reached 0.6 to 0.7. To the culture was added 0.65 mM IPTG as a protein expression inducer, and the culture was incubated at 37° C. for additional 3 hours. This culture was centrifuged at 4° C. at a speed of 4,000×g for 20 minutes to remove the supernatant and the bacterial cells were harvested. The harvested cells were suspended in a lysis buffer (100 mM NaH₂PO₄, 10 mM Tris-HCl, 8 M urea, pH 8.0) and the suspension was subjected to sonication to disrupt the cells. The cell lysates were centrifuged at a speed of 14,000×g for 15 minutes to separate the insoluble fraction from the soluble fraction. The soluble and insoluble fractions thus obtained were loaded on an SDS-PAGE gel separately to analyze the protein expression profile and the degree of expression.

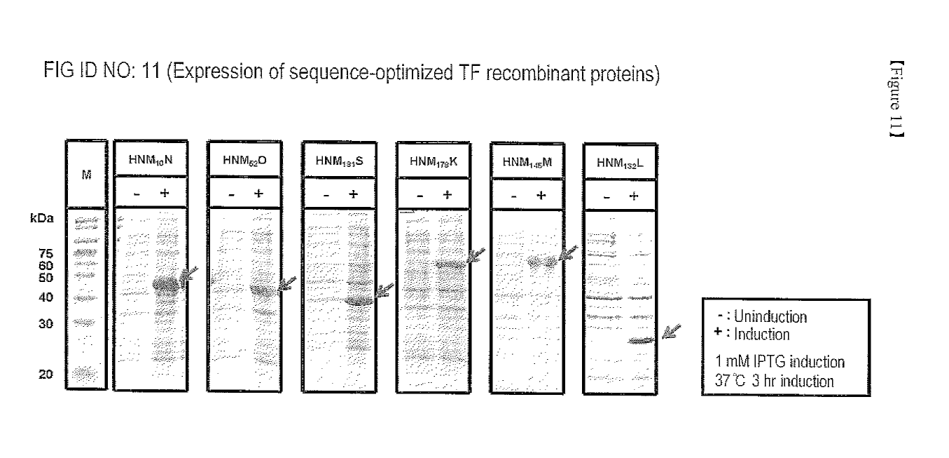

As a result, as shown in FIG. 11, the cell permeable reprogramming transcription factor recombinant proteins according to the present invention obtained from the expression vectors containing the recombinant protein gene constructs illustrated in FIG. 10 are mostly present in the insoluble fraction as inclusion bodies (about 34 kDa of Nanog, about 40 kDa of Oct4, about 35 kDa of Sox2, about 52 kDa of Klf4, and about 50 kDa of cMyc). Further, the expression of the target protein was found to be significantly increased in the culture solution with IPTG (+) compared to that without IPTG (−),when compared to the expression of the protein prior to the codon optimization.

### EFFECT OF THE INVENTION

The cell permeable reprogramming transcription factor recombinant proteins according to the present invention can introduce reprogramming transcription factors into a nucleus of a cell with high efficiency. The recombinant protein of the present invention can solve the problems of prior art caused by insertion of exogenous genes and is useful in the establishment of induced pluripotent stem cells with high efficiency.

1-25. (canceled)

26. A cell permeable reprogramming transcription factor recombinant protein comprising a macromolecule transduction domain (MTD) fused to one terminus or both termini of a reprogramming transcription factor,
wherein
the reprogramming transcription factor has an amino acid sequence selected from the group consisting of SEQ ID NOs: 2, 4, 6, 8, 10, and 12; and
the MTD has an amino acid sequence selected from the group consisting of SEQ ID NOs: 16 and 18.

27. The cell permeable reprogramming transcription factor recombinant protein according to claim 26, wherein the MTD is a JO-84 MTD having an amino acid sequence represented by SEQ ID NO: 16 or a JO-86 MTD having an amino acid sequence represented by SEQ ID NO: 18.

28. The cell permeable reprogramming transcription recombinant protein of claim 26, wherein the recombinant protein comprises at least one selected from a nuclear localization sequence (NLS) and a histidine-tag affinity domain fused to either terminus.

29. The cell permeable reprogramming transcription factor recombinant protein according to claim 26 selected from the group consisting of:
a recombinant protein wherein a JO-84 MTD having an amino acid sequence represented by SEQ ID NO: 16 is fused to the N-terminus of a reprogramming transcription factor having an amino acid sequence selected from the group consisting of SEQ ID NOs: 2, 4, 6, 8, 10, and 12;
a recombinant protein wherein a JO-84 MTD having an amino acid sequence represented by SEQ ID NO: 16 is fused to the C-terminus of a reprogramming transcription factor having an amino acid sequence selected from the group consisting of SEQ ID NOs: 2, 4, 6, 8, 10, and 12;
a recombinant protein wherein a JO-84 MTD having an amino acid sequence represented by SEQ ID NO: 16 is fused to both termini of a reprogramming transcription factor having an amino acid sequence selected from the group consisting of SEQ ID NOs: 2, 4, 6, 8, 10, and 12;
a recombinant protein wherein a JO-86 MTD having an amino acid sequence represented by SEQ ID NO: 18 is fused to the N-terminus of a reprogramming transcription factor having an amino acid sequence selected from the group consisting of SEQ ID NOs: 2, 4, 6, 8, 10, and 12;
a recombinant protein wherein a JO-86 MTD having an amino acid sequence represented by SEQ ID NO: 18 is fused to the C-terminus of a reprogramming transcription factor having an amino acid sequence selected from the group consisting of SEQ ID NOs: 2, 4, 6, 8, 10, and 12; and
a recombinant protein wherein a JO-86 MTD having an amino acid sequence represented by SEQ ID NO: 18 is fused to both termini of a reprogramming transcription factor having an amino acid sequence selected from the group consisting of SEQ ID NOs: 2, 4, 6, 8, 10, and 12.

30. The cell permeable reprogramming transcription factor recombinant protein according to claim 26, wherein the recombinant protein has an amino acid sequence selected from the group consisting of:
SEQ ID NOs: 22, 24, 26, 28, 30, or 32 for Nanog;
SEQ ID NOs: 50, 52, 54, 56, 58, or 60 for Oct4;
SEQ ID NOs: 78, 80, 82, 84, 86, or 88 for Sox2;
SEQ ID NOs: 106, 108, 110, 112, 114, or 116 for Klf4;
SEQ ID NOs: 134, 136, 138, 140, 142, or 144 for cMyc; and
SEQ ID NOs: 162, 164, 166, 168, 170, or 172 for Lin28.

31. The cell permeable reprogramming transcription factor recombinant protein according to claim 26, wherein the recombinant protein has an amino acid sequence selected from the group consisting of:
SEQ ID NOs: 36, 38, 40, 42, 44, or 46 for Nanog;
SEQ ID NOs: 64, 66, 68, 70, 72, or 74 for Oct4;
SEQ ID NOs: 92, 94, 96, 98, 100, or 102 for Sox2;
SEQ ID NOs: 120, 122, 124, 126, 128, or 130 for Klf4;
SEQ ID NOs: 148, 150, 152, 154, 156, or 158 for cMyc; and
SEQ ID NOs: 176, 178, 180, 182, 184, or 186 for Lin28.

32. A polynucleotide encoding the cell permeable reprogramming transcription factor recombinant protein according to claim 26.

33. The polynucleotide according to claim 32, wherein the polynucleotide has a nucleotide sequence selected from the group consisting of:
SEQ ID NOs: 21, 23, 25, 27, 29, or 31 for Nanog;
SEQ ID NOs: 49, 51, 53, 55, 57, or 59 for Oct4;
SEQ ID NOs: 77, 79, 81, 83, 85, or 87 for Sox2;
SEQ ID NOs: 105, 107, 109, 111, 113, or 115 for Klf4
SEQ ID NOs: 133, 135, 137, 139, 141, or 143 for cMyc; and
SEQ ID NOs: 161, 163, 165, 167, 169, or 171 for Lin28.

34. The polynucleotide according to claim 32, wherein the polynucleotide has a nucleotide sequence selected from the group consisting of:
SEQ ID NOs: 35, 37, 39, 41, 43, or 45 for Nanog;
SEQ ID NOs: 63, 65, 67, 69, 71, or 73 for Oct4;
SEQ ID NOs: 91, 93, 95, 97, 99, or 101 for Sox2;
SEQ ID NOs: 119, 121, 123, 125, 127, or 129 for Klf4;
SEQ ID NOs: 147, 149, 151, 153, 155, or 157 for cMyc; and
SEQ ID NOs: 175, 177, 179, 181, 183, or 185 for Lin28.

35. A recombinant expression vector comprising the polynucleotide according to claim 32.

36. The recombinant expression vector according to claim 35, wherein the expression vector is selected from the group consisting of pET28a(+)-HNM₈₄Nanog, pET28a(+)-HNNanogM₈₄, pET28a(+)-HNM₈₄NanogM₈₄, pET28a(+)-HNM₈₆Nanog, pET28a(+)-HNNanogM₈₆, pET28a(+)-HNM₈₆NanogM₈₆, pET28a(+)-HNM₈₄Oct4, pET28a(+)-HNOct4M₈₄, pET28a(+)-HNM₈₄Oct4M₈₄, pET28a(+)-HNM₈₆Oct4, pET28a(+)-HNOct4M₈₆, pET28a(+)-HNM₈₆Oct4M₈₆, pET28a(+)-HNM₈₄Sox2, pET28a(+)-HNSox2M₈₄, pET28a(+)-HNM₈₄Sox2M₈₄, pET28a(+)-HNM₈₆Sox2, pET28a(+)-HNSox2M₈₆, pET28a(+)-HNM₈₆Sox2M₈₆, pET28a(+)-HNM₈₄Klf4, pET28a(+)-HNKlf4M₈₄, pET28a(+)-HNM₈₄Klf4M₈₄, pET28a(+)-HNM₈₆Klf4, pET28a(+)-HNKlf4M₈₆, pET28a(+)-HNM₈₆Klf4M₈₆, pET28a(+)-HNM₈₄cMyc, pET28a(+)-HNcMycM₈₄, pET28a(+)-HNM₈₄cMycM₈₄, pET28a(+)-HNM₈₆cMyc, pET28a(+)-HNcMycM₈₆, pET28a(+)-HNM₈₆cMycM₈₆, pET28a(+)-HNM₈₄Lin28, pET28a(+)-HNLin28M₈₄, pET28a(+)-HNM₈₄Lin28M₈₄, pET28a(+)-HNM₈₆Lin28, pET28a(+)-HNLin28M₈₆, and pET28a(+)-HNM₈₆Lin28M₈₆.

37. A transformant which is obtained by transformation with the recombinant expression vector according to claim 35.

38. The transformant according to claim 37, wherein the transformant is E. coli DH5α/HNM₈₆Oct4 (KCTC 11640BP) or DH5α/HNM₈₆cMyc (KCTC 11661BP).

39. A method of producing the cell permeable reprogramming transcription factor recombinant protein comprising the steps of:
1) culturing the transformant according to claim 37 so that the cell permeable reprogramming transcription factor recombinant protein is expressed; and
2) recovering the expressed cell permeable reprogramming transcription factor recombinant protein from the culture,
wherein
the cell permeable reprogramming transcription factor recombinant protein comprising a macromolecule transduction domain (MTD) fused to one terminus or both termini of a reprogramming transcription factor,
wherein
the reprogramming transcription factor has an amino acid sequence selected from the group consisting of SEQ ID NOs: 2, 4, 6, 8, 10, and 12; and
the MTD has an amino acid sequence selected from the group consisting of SEQ ID NOs: 16 and 18.

40. A cell permeable reprogramming transcription factor recombinant protein comprising a macromolecule transduction domain (MTD) fused to the recombinant protein, wherein
the MTD having an amino acid sequence represented by SEQ ID NO: 224 is fused to the N-terminus of SEQ ID NO: 2;
the MTD having an amino acid sequence represented by SEQ ID NO: 226 is fused to the N-terminus of SEQ ID NO: 4;
the MTD having an amino acid sequence represented by SEQ ID NO: 234 is fused to the N-terminus of SEQ ID NO: 6;
the MTD having an amino acid sequence represented by SEQ ID NO: 232 is fused to the N-terminus of SEQ ID NO: 8;
the MTD having an amino acid sequence represented by SEQ ID NO: 230 is fused to the N-terminus of SEQ ID NO: 10; or
the MTD having an amino acid sequence represented by SEQ ID NO: 228 is fused to the N-terminus of SEQ ID NO: 12.

41. The cell permeable reprogramming transcription factor recombinant protein according to claim 40, wherein the MTD is selected from the group consisting of:
a JO-10 MTD having an amino acid sequence represented by SEQ ID NO: 224;
a JO-52 MTD having an amino acid sequence represented by SEQ ID NO: 226;
a JO-132 MTD having an amino acid sequence represented by SEQ ID NO: 228;
a JO-145 MTD having an amino acid sequence represented by SEQ ID NO: 230;
a JO-173 MTD having an amino acid sequence represented by SEQ ID NO: 232; and
a JO-181 MTD having an amino acid sequence represented by SEQ ID NO: 234.

42. The cell permeable reprogramming transcription factor recombinant protein according to claim 40, wherein the recombinant protein comprises at least one selected from a nuclear localization sequence (NLS) or a histidine-tag affinity domain fused at either terminus of the recombinant protein.

43. The cell permeable reprogramming transcription factor recombinant protein according to claim 40,
wherein the recombinant protein has an amino acid sequence selected from the group consisting of SEQ ID NOs: 237, 240, 243, 246, 249, and 252.

44. A polynucleotide encoding the cell permeable reprogramming transcription factor recombinant protein according to claim 40.

45. The polynucleotide according to claim 44, characterized by having a nucleotide sequence selected from the group consisting of SEQ ID NOs: 236, 239, 242, 245, 248, and 251.

46. A recombinant expression vector comprising the polynucleotide according to claim 44.

47. The recombinant expression vector according to claim 46, wherein the recombinant expression vector is selected from the group consisting of pET28a(+)-HNM₁₀Nanog, pET28a(+)-HNM₅₂Oct4, pET28a(+)-HNM₁₈₁Sox2, pET28a(+)-HNM₁₇₃Klf4, pET28a(+)-HNM₁₄₅cMyc, and pET28a(+)-HNM₁₃₂Lin28.

48. A transformant which is obtained by transformation with the recombinant expression vector according to claim 46.

49. The transformant according to claim 48, wherein the transformant is selected from the group consisting of E. coli DH5α/HNM₁₀Nanog (KCTC 11660BP), DH5α/HNM₁₈₁Sox2 (KCTC 11659BP), DH5α/HNM₁₇₃Klf4 (KCTC 11662BP), DH5α/HNM₁₃₂Lin28 (KCTC 11663BP), DH5α/HNM₅₂Oct4, BL21 (DE3)/HNM₁₀Nanog, BL21 (DE3)/HNM₁₈₁Sox2, BL21 (DE3)/HNM₁₇₃Klf4, BL21 (DE3)/HNM₁₃₂Lin28, and BL21 (DE3)/HNM₅₂Oct4.

50. A method of producing the cell permeable reprogramming transcription factor recombinant protein comprising the steps of:
1) culturing the transformant according to claim 48 so that the cell permeable reprogramming transcription factor recombinant protein is expressed; and
2) recovering the expressed cell permeable reprogramming transcription factor recombinant protein from the culture,
wherein the cell permeable reprogramming transcription factor recombinant protein comprising a macromolecule transduction domain (MTD) fused to the recombinant protein,
wherein
the MTD having an amino acid sequence represented by SEQ ID NO: 224 is fused to the N-terminus of SEQ ID NO: 2;
the MTD having an amino acid sequence represented by SEQ ID NO: 226 is fused to the N-terminus of SEQ ID NO: 4;
the MTD having an amino acid sequence represented by SEQ ID NO: 234 is fused to the N-terminus of SEQ ID NO: 6;
the MTD having an amino acid sequence represented by SEQ ID NO: 232 is fused to the N-terminus of SEQ ID NO: 8;
the MTD having an amino acid sequence represented by SEQ ID NO: 230 is fused to the N-terminus of SEQ ID NO: 10; or
the MTD having an amino acid sequence represented by SEQ ID NO: 228 is fused to the N-terminus of SEQ ID NO: 12.


SEQUENCE LISTING (252 sequences)

SEQ ID NO: 1 - DNA, 918 bp, Artificial Sequence
  Nanog cDNA Sequence
atgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtga

SEQ ID NO: 2 - protein, 305 aa, Artificial Sequence
  Nanog Amino Acid Sequence
MSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 3 - DNA, 1083 bp, Artificial Sequence
  Human Oct4 cDNA Sequence
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 4 - protein, 360 aa, Artificial Sequence
  Human Oct4 Amino Acid Sequence
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 5 - DNA, 954 bp, Artificial Sequence
  Sox2 cDNA Sequence
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 6 - protein, 317 aa, Artificial Sequence
  Sox2 Amino Acid Sequence
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 7 - DNA, 1413 bp, Artificial Sequence
  Klf4 cDNA Sequence
atggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 8 - protein, 470 aa, Artificial Sequence
  Klf4 Amino Acid Sequence
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 9 - DNA, 1368 bp, Artificial Sequence
  cMyc cDNA Sequence
atgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 10 - protein, 455 aa, Artificial Sequence
  cMyc Amino Acid Sequence
MLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 11 - DNA, 630 bp, Artificial Sequence
  Lin28 cDNA Sequence
atgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattga

SEQ ID NO: 12 - protein, 209 aa, Artificial Sequence
  Lin28 Amino Acid Sequence
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 13 - DNA, 15 bp, Artificial Sequence
  SV40 large T antigen-derived NLS cDNA Sequence
aagaagaagaggaag

SEQ ID NO: 14 - protein, 5 aa, Artificial Sequence
  SV40 large T antigen-derived NLS Amino Acid Sequence
KKKRK

SEQ ID NO: 15 - DNA, 27 bp, Artificial Sequence
  JO-84 MTD cDNA Sequence
ctggtggcggcgctgctggcggtgctg

SEQ ID NO: 16 - protein, 9 aa, Artificial Sequence
  JO-84 MTD Amino Acid Sequence
LVAALLAVL

SEQ ID NO: 17 - DNA, 24 bp, Artificial Sequence
  JO-86 MTD cDNA Sequence
ctggcggtgctggcggcggcgccg

SEQ ID NO: 18 - protein, 8 aa, Artificial Sequence
  JO-86 MTD Amino Acid Sequence
LAVLAAAP

SEQ ID NO: 19 - DNA, 933 bp, Artificial Sequence
  NLS-Nanog cDNA Sequence
atgaagaagaagaggaagagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgtga

SEQ ID NO: 20 - protein, 310 aa, Artificial Sequence
  NLS-Nanog Amino Acid Sequence
MKKKRKSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 21 - DNA, 960 bp, Artificial Sequence
  NLS-JO-84 MTD-Nanog cDNA Sequence
atgaagaagaagaggaagctggtggcggcgctgctggcggtgctgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgtga

SEQ ID NO: 22 - protein, 319 aa, Artificial Sequence
  NLS-JO-84 MTD-Nanog Amino Acid Sequence
MKKKRKLVAALLAVLSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 23 - DNA, 960 bp, Artificial Sequence
  NLS-Nanog-JO-84 MTD cDNA Sequence
atgaagaagaagaggaagagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgctggtggcggcgctgctggcggtgctgtga

SEQ ID NO: 24 - protein, 319 aa, Artificial Sequence
  NLS-Nanog-JO-84 MTD Amino Acid Sequence
MKKKRKSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVLVAALLAVL

SEQ ID NO: 25 - DNA, 987 bp, Artificial Sequence
  NLS-JO-84 MTD-Nanog-JO-84 MTD cDNA Sequence
atgaagaagaagaggaagctggtggcggcgctgctggcggtgctgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgctggtggcggcgctgctggcggtgctgtga

SEQ ID NO: 26 - protein, 328 aa, Artificial Sequence
  NLS-JO-84 MTD-Nanog-JO-84 MTD Amino Acid Sequence
MKKKRKLVAALLAVLSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVLVAALLAVL

SEQ ID NO: 27 - DNA, 957 bp, Artificial Sequence
  NLS-JO-86 MTD-Nanog cDNA Sequence
atgaagaagaagaggaagctggcggtgctggcggcggcgccgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgtga

SEQ ID NO: 28 - protein, 318 aa, Artificial Sequence
  NLS-JO-86 MTD-Nanog Amino Acid Sequence
MKKKRKLAVLAAAPSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 29 - DNA, 957 bp, Artificial Sequence
  NLS-Nanog-JO-86 MTD cDNA Sequence
atgaagaagaagaggaagagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgctggcggtgctggcggcggcgccgtga

SEQ ID NO: 30 - protein, 318 aa, Artificial Sequence
  NLS-Nanog-JO-86 MTD Amino Acid Sequence
MKKKRKSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVLAVLAAAP

SEQ ID NO: 31 - DNA, 981 bp, Artificial Sequence
  NLS-JO-86 MTD-Nanog-JO-86 MTD cDNA Sequence
atgaagaagaagaggaagctggcggtgctggcggcggcgccgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgctggcggtgctggcggcggcgccgtga

SEQ ID NO: 32 - protein, 326 aa, Artificial Sequence
  NLS-JO-86 MTD-Nanog-JO-86 MTD Amino Acid Sequence
MKKKRKLAVLAAAPSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVLAVLAAAP

SEQ ID NO: 33 - DNA, 993 bp, Artificial Sequence
  His-NLS-Nanog cDNA Sequence
atgggcagcagccatcatcatcatcatcacagcagcggcctggtgccgcgcggcagccatatgaagaagaagaggaagagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgtga

SEQ ID NO: 34 - protein, 330 aa, Artificial Sequence
  His-NLS-Nanog Amino Acid Sequence
MGSSHHHHHHSSGLVPRGSHMKKKRKSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 35 - DNA, 1020 bp, Artificial Sequence
  His-NLS-JO-84 MTD-Nanog cDNA Sequence
atgggcagcagccatcatcatcatcatcacagcagcggcctggtgccgcgcggcagccatatgaagaagaagaggaagctggtggcggcgctgctggcggtgctgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgtga

SEQ ID NO: 36 - protein, 339 aa, Artificial Sequence
  His-NLS-JO-84 MTD-Nanog Amino Acid Sequence
MGSSHHHHHHSSGLVPRGSHMKKKRKLVAALLAVLSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 37 - DNA, 1020 bp, Artificial Sequence
  His-NLS-Nanog-JO-84 MTD cDNA Sequence
atgggcagcagccatcatcatcatcatcacagcagcggcctggtgccgcgcggcagccatatgaagaagaagaggaagagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgctggtggcggcgctgctggcggtgctgtga

SEQ ID NO: 38 - protein, 339 aa, Artificial Sequence
  His-NLS-Nanog-JO-84 MTD Amino Acid Sequence
MGSSHHHHHHSSGLVPRGSHMKKKRKSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVLVAALLAVL

SEQ ID NO: 39 - DNA, 1047 bp, Artificial Sequence
  His-NLS-JO-84 MTD-Nanog-JO-84 MTD cDNA Sequence
atgggcagcagccatcatcatcatcatcacagcagcggcctggtgccgcgcggcagccatatgaagaagaagaggaagctggtggcggcgctgctggcggtgctgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgctggtggcggcgctgctggcggtgctgtga

SEQ ID NO: 40 - protein, 348 aa, Artificial Sequence
  His-NLS-JO-84 MTD-Nanog-JO-84 MTD Amino Acid Sequence
MGSSHHHHHHSSGLVPRGSHMKKKRKLVAALLAVLSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVLVAALLAVL

SEQ ID NO: 41 - DNA, 1017 bp, Artificial Sequence
  His-NLS- JO-86 MTD -Nanog cDNA Sequence
atgggcagcagccatcatcatcatcatcacagcagcggcctggtgccgcgcggcagccatatgaagaagaagaggaagctggcggtgctggcggcggcgccgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgtga

SEQ ID NO: 42 - protein, 338 aa, Artificial Sequence
  His-NLS-JO-86 MTD-Nanog Amino Acid Sequence
MGSSHHHHHHSSGLVPRGSHMKKKRKLAVLAAAPSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 43 - DNA, 1017 bp, Artificial Sequence
  His-NLS-Nanog-JO-86 MTD cDNA Sequence
atgggcagcagccatcatcatcatcatcacagcagcggcctggtgccgcgcggcagccatatgaagaagaagaggaagagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgctggcggtgctggcggcggcgccgtga

SEQ ID NO: 44 - protein, 338 aa, Artificial Sequence
  His-NLS-Nanog-JO-86 MTD Amino Acid Sequence
MGSSHHHHHHSSGLVPRGSHMKKKRKSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVLAVLAAAP

SEQ ID NO: 45 - DNA, 1041 bp, Artificial Sequence
  His-NLS-JO-86 MTD-Nanog-JO-86 MTD cDNA Sequence
atgggcagcagccatcatcatcatcatcacagcagcggcctggtgccgcgcggcagccatatgaagaagaagaggaagctggcggtgctggcggcggcgccgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgctggcggtgctggcggcggcgccgtga

SEQ ID NO: 46 - protein, 346 aa, Artificial Sequence
  His-NLS-JO-86 MTD-Nanog-JO-86 MTD Amino Acid Sequence
MGSSHHHHHHSSGLVPRGSHMKKKRKLAVLAAAPSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDVLAVLAAAP

SEQ ID NO: 47 - DNA, 1098 bp, Artificial Sequence
  NLS-Oct4 cDNA Sequence
atgaagaagaagaggaaggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 48 - protein, 365 aa, Artificial Sequence
  NLS-Oct4 Amino Acid Sequence
MKKKRKAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 49 - DNA, 1125 bp, Artificial Sequence
  NLS-JO-84 MTD-Oct4 cDNA Sequence
atgaagaagaagaggaagctggtggcggcgctgctggcggtgctggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 50 - protein, 374 aa, Artificial Sequence
  NLS-JO-84 MTD-Oct4 Amino Acid Sequence
MKKKRKLVAALLAVLAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 51 - DNA, 1125 bp, Artificial Sequence
  NLS-Oct4-JO-84 MTD cDNA Sequence
atgaagaagaagaggaaggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacctggtggcggcgctgctggcggtgctgtga

SEQ ID NO: 52 - protein, 374 aa, Artificial Sequence
  NLS-Oct4-JO-84 MTD Amino Acid Sequence
MKKKRKAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNLVAALLAVL

SEQ ID NO: 53 - DNA, 1152 bp, Artificial Sequence
  NLS-JO-84 MTD-Oct4-JO-84 MTD cDNA Sequence
atgaagaagaagaggaagctggtggcggcgctgctggcggtgctggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacctggtggcggcgctgctggcggtgctgtga

SEQ ID NO: 54 - protein, 383 aa, Artificial Sequence
  NLS-JO-84 MTD-Oct4-JO-84 MTD Amino Acid Sequence
MKKKRKLVAALLAVLAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNLVAALLAVL

SEQ ID NO: 55 - DNA, 1122 bp, Artificial Sequence
  NLS- JO-86 MTD-Oct4 cDNA Sequence
atgaagaagaagaggaagctggcggtgctggcggcggcgccggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 56 - protein, 373 aa, Artificial Sequence
  NLS-JO-86 MTD-Oct4 Amico Acid Sequence
MKKKRKLAVLAAAPAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 57 - DNA, 1122 bp, Artificial Sequence
  NLS-Oct4-JO-86 MTD cDNA Sequence
atgaagaagaagaggaaggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacctggcggtgctggcggcggcgccgtga

SEQ ID NO: 58 - protein, 373 aa, Artificial Sequence
  NLS-Oct4-JO-86 MTD Amino Acid Sequence
MKKKRKAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNLAVLAAAP

SEQ ID NO: 59 - DNA, 1146 bp, Artificial Sequence
  NLS-JO-86 MTD-Oct4-JO-86 MTD cDNA Sequence
atgaagaagaagaggaagctggcggtgctggcggcggcgccggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacctggcggtgctggcggcggcgccgtga

SEQ ID NO: 60 - protein, 381 aa, Artificial Sequence
  NLS-JO-86 MTD-Oct4-JO-86 MTD Amino Acid Sequence
MKKKRKLAVLAAAPAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNLAVLAAAP

SEQ ID NO: 61 - DNA, 1158 bp, Artificial Sequence
  His-NLS-Oct4 cDNA Sequence
atgggcagcagccatcatcatcatcatcacagcagcggcctggtgccgcgcggcagccatatgaagaagaagaggaaggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 62 - protein, 385 aa, Artificial Sequence
  His-NLS-Oct4 Amino Acid Sequence
MGSSHHHHHHSSGLVPRGSHMKKKRKAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 63 - DNA, 1185 bp, Artificial Sequence
  His-NLS-JO-84 MTD-Oct4 cDNA Sequence
atgggcagcagccatcatcatcatcatcacagcagcggcctggtgccgcgcggcagccatatgaagaagaagaggaagctggtggcggcgctgctggcggtgctggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 64 - protein, 394 aa, Artificial Sequence
  His-NLS-JO-84 MTD-Oct4 Amino Acid Sequence
MGSSHHHHHHSSGLVPRGSHMKKKRKLVAALLAVLAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 65 - DNA, 1185 bp, Artificial Sequence
  His-NLS-Oct4-JO-84 MTD cDNA Sequence
atgggcagcagccatcatcatcatcatcacagcagcggcctggtgccgcgcggcagccatatgaagaagaagaggaaggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacctggtggcggcgctgctggcggtgctgtga

SEQ ID NO: 66 - protein, 394 aa, Artificial Sequence
  His-NLS-Oct4-JO-84 MTD Amino Acid Sequence
MGSSHHHHHHSSGLVPRGSHMKKKRKAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNLVAALLAVL

SEQ ID NO: 67 - DNA, 1212 bp, Artificial Sequence
  His-NLS-JO-84 MTD-Oct4-JO-84 MTD cDNA Sequence
atgggcagcagccatcatcatcatcatcacagcagcggcctggtgccgcgcggcagccatatgaagaagaagaggaagctggtggcggcgctgctggcggtgctggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacctggtggcggcgctgctggcggtgctgtga

SEQ ID NO: 68 - protein, 403 aa, Artificial Sequence
  His-NLS-JO-84 MTD-Oct4-JO-84 MTD Amino Acid Sequence
MGSSHHHHHHSSGLVPRGSHMKKKRKLVAALLAVLAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNLVAALLAVL

SEQ ID NO: 69 - DNA, 1182 bp, Artificial Sequence
  His-NLS-JO-86 MTD-Oct4 cDNA Sequence
atgggcagcagccatcatcatcatcatcacagcagcggcctggtgccgcgcggcagccatatgaagaagaagaggaagctggcggtgctggcggcggcgccggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 70 - protein, 393 aa, Artificial Sequence
  His-NLS-JO-86 MTD-Oct4 Amico Acid Sequence
MGSSHHHHHHSSGLVPRGSHMKKKRKLAVLAAAPAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 71 - DNA, 1182 bp, Artificial Sequence
  His-NLS-Oct4-JO-86 MTD cDNA Sequence
atgggcagcagccatcatcatcatcatcacagcagcggcctggtgccgcgcggcagccatatgaagaagaagaggaaggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacctggcggtgctggcggcggcgccgtga

SEQ ID NO: 72 - protein, 393 aa, Artificial Sequence
  His-NLS-Oct4-JO-86 MTD Amino Acid Sequence
MGSSHHHHHHSSGLVPRGSHMKKKRKAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNLAVLAAAP

SEQ ID NO: 73 - DNA, 1206 bp, Artificial Sequence
  His-NLS-JO-86 MTD-Oct4-JO-86 MTD cDNA Sequence
atgggcagcagccatcatcatcatcatcacagcagcggcctggtgccgcgcggcagccatatgaagaagaagaggaagctggcggtgctggcggcggcgccggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacctggcggtgctggcggcggcgccgtga

SEQ ID NO: 74 - protein, 401 aa, Artificial Sequence
  His-NLS-JO-86 MTD-Oct4-JO-86 MTD Amino Acid Sequence
MGSSHHHHHHSSGLVPRGSHMKKKRKLAVLAAAPAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSNLAVLAAAP

SEQ ID NO: 75 - DNA, 969 bp, Artificial Sequence
  NLS-Sox2 cDNA Sequence
atgaagaagaagaggaagtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 76 - protein, 322 aa, Artificial Sequence
  NLS-Sox2 Amino Acid Sequence
MKKKRKYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 77 - DNA, 996 bp, Artificial Sequence
  NLS-JO-84 MTD-Sox2 cDNA Sequence
atgaagaagaagaggaagctggtggcggcgctgctggcggtgctgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 78 - protein, 331 aa, Artificial Sequence
  NLS-JO-84 MTD-Sox2 Amino Acid Sequence
MKKKRKLVAALLAVLYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 79 - DNA, 996 bp, Artificial Sequence
  NLS-Sox2-JO-84 MTD cDNA Sequence
atgaagaagaagaggaagtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgctggtggcggcgctgctggcggtgctgtga

SEQ ID NO: 80 - protein, 331 aa, Artificial Sequence
  NLS-Sox2-JO-84 MTD Amino Acid Sequence
MKKKRKYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMLVAALLAVL

SEQ ID NO: 81 - DNA, 1023 bp, Artificial Sequence
  NLS-JO-84 MTD-Sox2-JO-84 MTD cDNA Sequence
atgaagaagaagaggaagctggtggcggcgctgctggcggtgctgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgctggtggcggcgctgctggcggtgctgtga

SEQ ID NO: 82 - protein, 340 aa, Artificial Sequence
  NLS-JO-84 MTD-Sox2-JO-84 MTD Amino Acid Sequence
MKKKRKLVAALLAVLYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMLVAALLAVL

SEQ ID NO: 83 - DNA, 993 bp, Artificial Sequence
  NLS-JO-86 MTD-Sox2 cDNA Sequence
atgaagaagaagaggaagctggcggtgctggcggcggcgccgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 84 - protein, 330 aa, Artificial Sequence
  NLS-JO-86 MTD-Sox2 Amino Acid Sequence
MKKKRKLAVLAAAPYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 85 - DNA, 993 bp, Artificial Sequence
  NLS-Sox2-JO-86 MTD cDNA Sequence
atgaagaagaagaggaagtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgctggcggtgctggcggcggcgccgtga

SEQ ID NO: 86 - protein, 330 aa, Artificial Sequence
  NLS-Sox2-JO-86 MTD Amino Acid Sequence
MKKKRKYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMLAVLAAAP

SEQ ID NO: 87 - DNA, 1017 bp, Artificial Sequence
  NLS-JO-86 MTD-Sox2-JO-86 MTD cDNA Sequence
atgaagaagaagaggaagctggcggtgctggcggcggcgccgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgctggcggtgctggcggcggcgccgtga

SEQ ID NO: 88 - protein, 338 aa, Artificial Sequence
  NLS-JO-86 MTD-Sox2-JO-86 MTD Amino Acid Sequence
MKKKRKLAVLAAAPYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMLAVLAAAP

SEQ ID NO: 89 - DNA, 1029 bp, Artificial Sequence
  His-NLS-Sox2 cDNA Sequence
atgggcagcagccatcatcatcatcatcacagcagcggcctggtgccgcgcggcagccatatgaagaagaagaggaagtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 90 - protein, 342 aa, Artificial Sequence
  His-NLS-Sox2 Amino Acid Sequence
MGSSHHHHHHSSGLVPRGSHMKKKRKYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 91 - DNA, 1056 bp, Artificial Sequence
  His-NLS-JO-84 MTD-Sox2 cDNA Sequence
atgggcagcagccatcatcatcatcatcacagcagcggcctggtgccgcgcggcagccatatgaagaagaagaggaagctggtggcggcgctgctggcggtgctgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 92 - protein, 351 aa, Artificial Sequence
  His-NLS-JO-84 MTD-Sox2 Amino Acid Sequence
MGSSHHHHHHSSGLVPRGSHMKKKRKLVAALLAVLYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 93 - DNA, 1056 bp, Artificial Sequence
  His-NLS-Sox2-JO-84 MTD cDNA Sequence
atgggcagcagccatcatcatcatcatcacagcagcggcctggtgccgcgcggcagccatatgaagaagaagaggaagtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgctggtggcggcgctgctggcggtgctgtga

SEQ ID NO: 94 - protein, 351 aa, Artificial Sequence
  His-NLS-Sox2-JO-84 MTD Amino Acid Sequence
MGSSHHHHHHSSGLVPRGSHMKKKRKYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMLVAALLAVL

SEQ ID NO: 95 - DNA, 1083 bp, Artificial Sequence
  His-NLS-JO-84 MTD-Sox2-JO-84 MTD cDNA Sequence
atgggcagcagccatcatcatcatcatcacagcagcggcctggtgccgcgcggcagccatatgaagaagaagaggaagctggtggcggcgctgctggcggtgctgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgctggtggcggcgctgctggcggtgctgtga

SEQ ID NO: 96 - protein, 360 aa, Artificial Sequence
  His-NLS-JO-84 MTD-Sox2-JO-84 MTD Amino Acid Sequence
MGSSHHHHHHSSGLVPRGSHMKKKRKLVAALLAVLYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMLVAALLAVL

SEQ ID NO: 97 - DNA, 1053 bp, Artificial Sequence
  His-NLS-JO-86 MTD-Sox2 cDNA Sequence
atgggcagcagccatcatcatcatcatcacagcagcggcctggtgccgcgcggcagccatatgaagaagaagaggaagctggcggtgctggcggcggcgccgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 98 - protein, 350 aa, Artificial Sequence
  His-NLS-JO-86 MTD-Sox2 Amino Acid Sequence
MGSSHHHHHHSSGLVPRGSHMKKKRKLAVLAAAPYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 99 - DNA, 1053 bp, Artificial Sequence
  His-NLS-Sox2-JO-86 MTD cDNA Sequence
atgggcagcagccatcatcatcatcatcacagcagcggcctggtgccgcgcggcagccatatgaagaagaagaggaagtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgctggcggtgctggcggcggcgccgtga

SEQ ID NO: 100 - protein, 350 aa, Artificial Sequence
  His-NLS-Sox2-JO-86 MTD Amino Acid Sequence
MGSSHHHHHHSSGLVPRGSHMKKKRKYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMLAVLAAAP

SEQ ID NO: 101 - DNA, 1077 bp, Artificial Sequence
  His-NLS-JO-86 MTD-Sox2-JO-86 MTD cDNA Sequence
atgggcagcagccatcatcatcatcatcacagcagcggcctggtgccgcgcggcagccatatgaagaagaagaggaagctggcggtgctggcggcggcgccgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgctggcggtgctggcggcggcgccgtga

SEQ ID NO: 102 - protein, 358 aa, Artificial Sequence
  His-NLS-JO-86 MTD-Sox2-JO-86 MTD Amino Acid Sequence
MGSSHHHHHHSSGLVPRGSHMKKKRKLAVLAAAPYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHMLAVLAAAP

SEQ ID NO: 103 - DNA, 1428 bp, Artificial Sequence
  NLS-Klf4 cDNA Sequence
atgaagaagaagaggaaggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 104 - protein, 475 aa, Artificial Sequence
  NLS-Klf4 Amino Acid Sequence
MKKKRKAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 105 - DNA, 1455 bp, Artificial Sequence
  NLS-JO-84 MTD-Klf4 cDNA Sequence
atgaagaagaagaggaagctggtggcggcgctgctggcggtgctggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 106 - protein, 484 aa, Artificial Sequence
  NLS-JO-84 MTD-Klf4 Amino Acid Sequence
MKKKRKLVAALLAVLAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 107 - DNA, 1455 bp, Artificial Sequence
  NLS-Klf4-JO-84 MTD cDNA Sequence
atgaagaagaagaggaaggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcattttctggtggcggcgctgctggcggtgctgtaa

SEQ ID NO: 108 - protein, 484 aa, Artificial Sequence
  NLS-Klf4-JO-84 MTD Amino Acid Sequence
MKKKRKAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFLVAALLAVL

SEQ ID NO: 109 - DNA, 1482 bp, Artificial Sequence
  NLS-JO-84 MTD-Klf4-JO-84 MTD cDNA Sequence
atgaagaagaagaggaagctggtggcggcgctgctggcggtgctggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcattttctggtggcggcgctgctggcggtgctgtaa

SEQ ID NO: 110 - protein, 493 aa, Artificial Sequence
  NLS-JO-84 MTD-Klf4-JO-84 MTD Amino Acid Sequence
MKKKRKLVAALLAVLAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFLVAALLAVL

SEQ ID NO: 111 - DNA, 1452 bp, Artificial Sequence
  NLS-JO-86 MTD-Klf4 cDNA Sequence
atgaagaagaagaggaagctggcggtgctggcggcggcgccggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 112 - protein, 483 aa, Artificial Sequence
  NLS-JO-86 MTD-Klf4 Amino Acid Sequence
MKKKRKLAVLAAAPAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 113 - DNA, 1452 bp, Artificial Sequence
  NLS-Klf4-JO-86 MTD cDNA Sequence
atgaagaagaagaggaaggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcattttctggcggtgctggcggcggcgccgtaa

SEQ ID NO: 114 - protein, 483 aa, Artificial Sequence
  NLS-Klf4-JO-86 MTD Amino Acid Sequence
MKKKRKAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFLAVLAAAP

SEQ ID NO: 115 - DNA, 1476 bp, Artificial Sequence
  NLS- JO-86 MTD-Klf4-JO-86 MTD cDNA Sequence
atgaagaagaagaggaagctggcggtgctggcggcggcgccggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcattttctggcggtgctggcggcggcgccgtaa

SEQ ID NO: 116 - protein, 491 aa, Artificial Sequence
  NLS-JO-86 MTD -Klf4-JO-86 MTD Amino Acid Sequence
MKKKRKLAVLAAAPAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFLAVLAAAP

SEQ ID NO: 117 - DNA, 1488 bp, Artificial Sequence
  His-NLS-Klf4 cDNA Sequence
atgggcagcagccatcatcatcatcatcacagcagcggcctggtgccgcgcggcagccatatgaagaagaagaggaaggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 118 - protein, 495 aa, Artificial Sequence
  His-NLS-Klf4 Amino Acid Sequence
MGSSHHHHHHSSGLVPRGSHMKKKRKAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 119 - DNA, 1515 bp, Artificial Sequence
  His-NLS-JO-84 MTD-Klf4 cDNA Sequence
atgggcagcagccatcatcatcatcatcacagcagcggcctggtgccgcgcggcagccatatgaagaagaagaggaagctggtggcggcgctgctggcggtgctggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 120 - protein, 504 aa, Artificial Sequence
  His-NLS-JO-84 MTD-Klf4 Amino Acid Sequence
MGSSHHHHHHSSGLVPRGSHMKKKRKLVAALLAVLAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 121 - DNA, 1515 bp, Artificial Sequence
  His-NLS-Klf4-JO-84 MTD cDNA Sequence
atgggcagcagccatcatcatcatcatcacagcagcggcctggtgccgcgcggcagccatatgaagaagaagaggaaggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcattttctggtggcggcgctgctggcggtgctgtaa

SEQ ID NO: 122 - protein, 504 aa, Artificial Sequence
  His-NLS-Klf4-JO-84 MTD Amino Acid Sequence
MGSSHHHHHHSSGLVPRGSHMKKKRKAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFLVAALLAVL

SEQ ID NO: 123 - DNA, 1542 bp, Artificial Sequence
  His-NLS-JO-84 MTD-Klf4-JO-84 MTD cDNA Sequence
atgggcagcagccatcatcatcatcatcacagcagcggcctggtgccgcgcggcagccatatgaagaagaagaggaagctggtggcggcgctgctggcggtgctggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcattttctggtggcggcgctgctggcggtgctgtaa

SEQ ID NO: 124 - protein, 513 aa, Artificial Sequence
  His-NLS-JO-84 MTD-Klf4-JO-84 MTD Amino Acid Sequence
MGSSHHHHHHSSGLVPRGSHMKKKRKLVAALLAVLAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFLVAALLAVL

SEQ ID NO: 125 - DNA, 1512 bp, Artificial Sequence
  His-NLS-JO-86 MTD -Klf4 cDNA Sequence
atgggcagcagccatcatcatcatcatcacagcagcggcctggtgccgcgcggcagccatatgaagaagaagaggaagctggcggtgctggcggcggcgccggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 126 - protein, 503 aa, Artificial Sequence
  His-NLS-JO-86 MTD -Klf4 Amino Acid Sequence
MGSSHHHHHHSSGLVPRGSHMKKKRKLAVLAAAPAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 127 - DNA, 1512 bp, Artificial Sequence
  His-NLS-Klf4-JO-86 MTD cDNA Sequence
atgggcagcagccatcatcatcatcatcacagcagcggcctggtgccgcgcggcagccatatgaagaagaagaggaaggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcattttctggcggtgctggcggcggcgccgtaa

SEQ ID NO: 128 - protein, 503 aa, Artificial Sequence
  His-NLS-Klf4-JO-86 MTD Amino Acid Sequence
MGSSHHHHHHSSGLVPRGSHMKKKRKAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFLAVLAAAP

SEQ ID NO: 129 - DNA, 1536 bp, Artificial Sequence
  His-NLS-JO-86 MTD-Klf4-JO-86 MTD cDNA Sequence
atgggcagcagccatcatcatcatcatcacagcagcggcctggtgccgcgcggcagccatatgaagaagaagaggaagctggcggtgctggcggcggcgccggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcattttctggcggtgctggcggcggcgccgtaa

SEQ ID NO: 130 - protein, 511 aa, Artificial Sequence
  His-NLS-JO-86 MTD-Klf4-JO-86 MTD Amino Acid Sequence
MGSSHHHHHHSSGLVPRGSHMKKKRKLAVLAAAPAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHFLAVLAAAP

SEQ ID NO: 131 - DNA, 1383 bp, Artificial Sequence
  NLS-cMyc cDNA Sequence
atgaagaagaagaggaagctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 132 - protein, 460 aa, Artificial Sequence
  NLS-cMyc Amino Acid Sequence
MKKKRKLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 133 - DNA, 1410 bp, Artificial Sequence
  NLS-JO-84 MTD-cMyc cDNA Sequence
atgaagaagaagaggaagctggtggcggcgctgctggcggtgctgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 134 - protein, 469 aa, Artificial Sequence
  NLS-JO-84 MTD-cMyc Amino Acid Sequence
MKKKRKLVAALLAVLLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 135 - DNA, 1410 bp, Artificial Sequence
  NLS-cMyc-JO-84 MTD cDNA Sequence
atgaagaagaagaggaagctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgctggtggcggcgctgctggcggtgctgtaa

SEQ ID NO: 136 - protein, 469 aa, Artificial Sequence
  NLS-cMyc-JO-84 MTD Amino Acid Sequence
MKKKRKLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCALVAALLAVL

SEQ ID NO: 137 - DNA, 1437 bp, Artificial Sequence
  NLS-JO-84 MTD-cMyc-JO-84 MTD cDNA Sequence
atgaagaagaagaggaagctggtggcggcgctgctggcggtgctgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgctggtggcggcgctgctggcggtgctgtaa

SEQ ID NO: 138 - protein, 478 aa, Artificial Sequence
  NLS-JO-84 MTD-cMyc-JO-84 MTD Amino Acid Sequence
MKKKRKLVAALLAVLLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCALVAALLAVL

SEQ ID NO: 139 - DNA, 1407 bp, Artificial Sequence
  NLS-JO-86 MTD-cMyc cDNA Sequence
atgaagaagaagaggaagctggcggtgctggcggcggcgccgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 140 - protein, 468 aa, Artificial Sequence
  NLS-JO-86 MTD-cMyc Amino Acid Sequence
MKKKRKLAVLAAAPLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 141 - DNA, 1407 bp, Artificial Sequence
  NLS-cMyc-JO-86 MTD cDNA Sequence
atgaagaagaagaggaagctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgctggcggtgctggcggcggcgccgtaa

SEQ ID NO: 142 - protein, 468 aa, Artificial Sequence
  NLS-cMyc-JO-86 MTD Amino Acid Sequence
MKKKRKLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCALAVLAAAP

SEQ ID NO: 143 - DNA, 1431 bp, Artificial Sequence
  NLS-JO-86 MTD-cMyc-JO-86 MTD cDNA Sequence
atgaagaagaagaggaagctggcggtgctggcggcggcgccgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgctggcggtgctggcggcggcgccgtaa

SEQ ID NO: 144 - protein, 476 aa, Artificial Sequence
  NLS-JO-86 MTD-cMyc-JO-86 MTD Amino Acid Sequence
MKKKRKLAVLAAAPLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCALAVLAAAP

SEQ ID NO: 145 - DNA, 1443 bp, Artificial Sequence
  His-NLS-cMyc cDNA Sequence
atgggcagcagccatcatcatcatcatcacagcagcggcctggtgccgcgcggcagccatatgaagaagaagaggaagctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 146 - protein, 480 aa, Artificial Sequence
  His-NLS-cMyc Amino Acid Sequence
MGSSHHHHHHSSGLVPRGSHMKKKRKLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 147 - DNA, 1470 bp, Artificial Sequence
  His-NLS-JO-84 MTD-cMyc cDNA Sequence
atgggcagcagccatcatcatcatcatcacagcagcggcctggtgccgcgcggcagccatatgaagaagaagaggaagctggtggcggcgctgctggcggtgctgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 148 - protein, 489 aa, Artificial Sequence
  His-NLS-JO-84 MTD-cMyc Amino Acid Sequence
MGSSHHHHHHSSGLVPRGSHMKKKRKLVAALLAVLLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 149 - DNA, 1470 bp, Artificial Sequence
  His-NLS-cMyc-JO-84 MTD cDNA Sequence
atgggcagcagccatcatcatcatcatcacagcagcggcctggtgccgcgcggcagccatatgaagaagaagaggaagctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgctggtggcggcgctgctggcggtgctgtaa

SEQ ID NO: 150 - protein, 489 aa, Artificial Sequence
  His-NLS-cMyc-JO-84 MTD Amino Acid Sequence
MGSSHHHHHHSSGLVPRGSHMKKKRKLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCALVAALLAVL

SEQ ID NO: 151 - DNA, 1497 bp, Artificial Sequence
  His-NLS-JO-84 MTD-cMyc-JO-84 MTD cDNA Sequence
atgggcagcagccatcatcatcatcatcacagcagcggcctggtgccgcgcggcagccatatgaagaagaagaggaagctggtggcggcgctgctggcggtgctgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgctggtggcggcgctgctggcggtgctgtaa

SEQ ID NO: 152 - protein, 498 aa, Artificial Sequence
  His-NLS-JO-84 MTD-cMyc-JO-84 MTD Amino Acid Sequence
MGSSHHHHHHSSGLVPRGSHMKKKRKLVAALLAVLLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCALVAALLAVL

SEQ ID NO: 153 - DNA, 1467 bp, Artificial Sequence
  His-NLS-JO-86 MTD-cMyc cDNA Sequence
atgggcagcagccatcatcatcatcatcacagcagcggcctggtgccgcgcggcagccatatgaagaagaagaggaagctggcggtgctggcggcggcgccgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 154 - protein, 488 aa, Artificial Sequence
  His-NLS-JO-86 MTD-cMyc Amino Acid Sequence
MGSSHHHHHHSSGLVPRGSHMKKKRKLAVLAAAPLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 155 - DNA, 1467 bp, Artificial Sequence
  His-NLS-cMyc-JO-86 MTD cDNA Sequence
atgggcagcagccatcatcatcatcatcacagcagcggcctggtgccgcgcggcagccatatgaagaagaagaggaagctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgctggcggtgctggcggcggcgccgtaa

SEQ ID NO: 156 - protein, 488 aa, Artificial Sequence
  His-NLS-cMyc-JO-86 MTD Amino Acid Sequence
MGSSHHHHHHSSGLVPRGSHMKKKRKLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCALAVLAAAP

SEQ ID NO: 157 - DNA, 1491 bp, Artificial Sequence
  His-NLS-JO-86 MTD-cMyc-JO-86 MTD cDNA Sequence
atgggcagcagccatcatcatcatcatcacagcagcggcctggtgccgcgcggcagccatatgaagaagaagaggaagctggcggtgctggcggcggcgccgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgctggcggtgctggcggcggcgccgtaa

SEQ ID NO: 158 - protein, 496 aa, Artificial Sequence
  His-NLS-JO-86 MTD-cMyc-JO-86 MTD Amino Acid Sequence
MGSSHHHHHHSSGLVPRGSHMKKKRKLAVLAAAPLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCALAVLAAAP

SEQ ID NO: 159 - DNA, 645 bp, Artificial Sequence
  NLS-Lin28 cDNA Sequence
atgaagaagaagaggaagggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattga

SEQ ID NO: 160 - protein, 214 aa, Artificial Sequence
  NLS-Lin28 Amino Acid Sequence
MKKKRKGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 161 - DNA, 672 bp, Artificial Sequence
  NLS-JO-84 MTD-Lin28 cDNA Sequence
atgaagaagaagaggaagctggtggcggcgctgctggcggtgctgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattga

SEQ ID NO: 162 - protein, 223 aa, Artificial Sequence
  NLS-JO-84 MTD-Lin28 Amino Acid Sequence
MKKKRKLVAALLAVLGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 163 - DNA, 672 bp, Artificial Sequence
  NLS-Lin28-JO-84 MTD cDNA Sequence
atgaagaagaagaggaagggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaatctggtggcggcgctgctggcggtgctgtga

SEQ ID NO: 164 - protein, 223 aa, Artificial Sequence
  NLS-Lin28-JO-84 MTD Amino Acid Sequence
MKKKRKGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNLVAALLAVL

SEQ ID NO: 165 - DNA, 699 bp, Artificial Sequence
  NLS-JO-84 MTD-Lin28-JO-84 MTD cDNA Sequence
atgaagaagaagaggaagctggtggcggcgctgctggcggtgctgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaatctggtggcggcgctgctggcggtgctgtga

SEQ ID NO: 166 - protein, 232 aa, Artificial Sequence
  NLS-JO-84 MTD-Lin28-JO-84 MTD Amino Acid Sequence
MKKKRKLVAALLAVLGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNLVAALLAVL

SEQ ID NO: 167 - DNA, 669 bp, Artificial Sequence
  NLS-JO-86 MTD -Lin28 cDNA Sequence
atgaagaagaagaggaagctggcggtgctggcggcggcgccgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattga

SEQ ID NO: 168 - protein, 222 aa, Artificial Sequence
  NLS-JO-86 MTD -Lin28 Amino Acid Sequence
MKKKRKLAVLAAAPGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 169 - DNA, 669 bp, Artificial Sequence
  NLS-Lin28-JO-86 MTD cDNA Sequence
atgaagaagaagaggaagggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaatctggcggtgctggcggcggcgccgtga

SEQ ID NO: 170 - protein, 222 aa, Artificial Sequence
  NLS-Lin28-JO-86 MTD Amino Acid Sequence
MKKKRKGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNLAVLAAAP

SEQ ID NO: 171 - DNA, 693 bp, Artificial Sequence
  NLS-JO-86 MTD-Lin28-JO-86 MTD cDNA Sequence
atgaagaagaagaggaagctggcggtgctggcggcggcgccgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaatctggcggtgctggcggcggcgccgtga

SEQ ID NO: 172 - protein, 230 aa, Artificial Sequence
  NLS-JO-86 MTD-Lin28-JO-86 MTD Amino Acid Sequence
MKKKRKLAVLAAAPGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNLAVLAAAP

SEQ ID NO: 173 - DNA, 705 bp, Artificial Sequence
  His-NLS-Lin28 cDNA Sequence
atgggcagcagccatcatcatcatcatcacagcagcggcctggtgccgcgcggcagccatatgaagaagaagaggaagggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattga

SEQ ID NO: 174 - protein, 234 aa, Artificial Sequence
  His-NLS-Lin28 Amino Acid Sequence
MGSSHHHHHHSSGLVPRGSHMKKKRKGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 175 - DNA, 732 bp, Artificial Sequence
  His-NLS-JO-84 MTD-Lin28 cDNA Sequence
atgggcagcagccatcatcatcatcatcacagcagcggcctggtgccgcgcggcagccatatgaagaagaagaggaagctggtggcggcgctgctggcggtgctgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattga

SEQ ID NO: 176 - protein, 243 aa, Artificial Sequence
  His-NLS-JO-84 MTD-Lin28 Amino Acid Sequence
MGSSHHHHHHSSGLVPRGSHMKKKRKLVAALLAVLGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 177 - DNA, 732 bp, Artificial Sequence
  His-NLS-Lin28-JO-84 MTD cDNA Sequence
atgggcagcagccatcatcatcatcatcacagcagcggcctggtgccgcgcggcagccatatgaagaagaagaggaagggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaatctggtggcggcgctgctggcggtgctgtga

SEQ ID NO: 178 - protein, 243 aa, Artificial Sequence
  His-NLS-Lin28-JO-84 MTD Amino Acid Sequence
MGSSHHHHHHSSGLVPRGSHMKKKRKGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNLVAALLAVL

SEQ ID NO: 179 - DNA, 759 bp, Artificial Sequence
  His-NLS-JO-84 MTD-Lin28-JO-84 MTD cDNA Sequence
atgggcagcagccatcatcatcatcatcacagcagcggcctggtgccgcgcggcagccatatgaagaagaagaggaagctggtggcggcgctgctggcggtgctgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaatctggtggcggcgctgctggcggtgctgtga

SEQ ID NO: 180 - protein, 252 aa, Artificial Sequence
  His-NLS-JO-84 MTD-Lin28-JO-84 MTD Amino Acid Sequence
MGSSHHHHHHSSGLVPRGSHMKKKRKLVAALLAVLGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNLVAALLAVL

SEQ ID NO: 181 - DNA, 729 bp, Artificial Sequence
  His-NLS-JO-86 MTD-Lin28 cDNA Sequence
atgggcagcagccatcatcatcatcatcacagcagcggcctggtgccgcgcggcagccatatgaagaagaagaggaagctggcggtgctggcggcggcgccgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattga

SEQ ID NO: 182 - protein, 242 aa, Artificial Sequence
  His-NLS-JO-86 MTD-Lin28 Amino Acid Sequence
MGSSHHHHHHSSGLVPRGSHMKKKRKLAVLAAAPGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 183 - DNA, 729 bp, Artificial Sequence
  His-NLS-Lin28-JO-86 MTD cDNA Sequence
atgggcagcagccatcatcatcatcatcacagcagcggcctggtgccgcgcggcagccatatgaagaagaagaggaagggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaatctggcggtgctggcggcggcgccgtga

SEQ ID NO: 184 - protein, 242 aa, Artificial Sequence
  His-NLS-Lin28-JO-86 MTD Amino Acid Sequence
MGSSHHHHHHSSGLVPRGSHMKKKRKGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNLAVLAAAP

SEQ ID NO: 185 - DNA, 753 bp, Artificial Sequence
  His-NLS -JO-86 MTD-Lin28-JO-86 MTD cDNA Sequence
atgggcagcagccatcatcatcatcatcacagcagcggcctggtgccgcgcggcagccatatgaagaagaagaggaagctggcggtgctggcggcggcgccgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaatctggcggtgctggcggcggcgccgtga

SEQ ID NO: 186 - protein, 250 aa, Artificial Sequence
  His-NLS -JO-86 MTD -Lin28-JO-86 MTD Amino Acid Sequence)
MGSSHHHHHHSSGLVPRGSHMKKKRKLAVLAAAPGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQNLAVLAAAP

SEQ ID NO: 187 - DNA, 45 bp, Artificial Sequence
  HhN-5′
ccgcatatgaagaagaagaggaagagtgtggatccagcttgtccc

SEQ ID NO: 188 - DNA, 36 bp, Artificial Sequence
  HhN-3′
ccgcatatgtcacacgtcttcaggttgcatgttcat

SEQ ID NO: 189 - DNA, 75 bp, Artificial Sequence
  HM1hN-5′
ccgcatatgaagaagaagaggaagctggtggcggcgctgctggcggtgctgagtgtggatccagcttgtccccaa

SEQ ID NO: 190 - DNA, 63 bp, Artificial Sequence
  HhNM1-3′
ccgcatatgtcacagcaccgccagcagcgccgccaccagcacgtcttcaggttgcatgttcat

SEQ ID NO: 191 - DNA, 72 bp, Artificial Sequence
  HM2hN-5′
ccgcatatgaagaagaagaggaagctggcggtgctggcggcggcgccgagtgtggatccagcttgtccccaa

SEQ ID NO: 192 - DNA, 60 bp, Artificial Sequence
  HhNM2-3′
ccgcatatgtcacggcgccgccgccagcaccgccagcacgtcttcaggttgcatgttcat

SEQ ID NO: 193 - DNA, 45 bp, Artificial Sequence
  HhO-5′
ccgcatatgaagaagaagaggaaggcgggacacctggcttcggat

SEQ ID NO: 194 - DNA, 36 bp, Artificial Sequence
  HhO-3′
ccgcatatgtcagtttgaatgcatgggagagcccag

SEQ ID NO: 195 - DNA, 75 bp, Artificial Sequence
  HM1hO-5′
ccgcatatgaagaagaagaggaagctggtggcggcgctgctggcggtgctggcgggacacctggcttcggatttc

SEQ ID NO: 196 - DNA, 63 bp, Artificial Sequence
  HhOM1-3′
ccgcatatgtcacagcaccgccagcagcgccgccaccaggtttgaatgcatgggagagcccag

SEQ ID NO: 197 - DNA, 72 bp, Artificial Sequence
  HM2hO-5′
ccgcatatgaagaagaagaggaagctggcggtgctggcggcggcgccggcgggacacctggcttcggatttc

SEQ ID NO: 198 - DNA, 60 bp, Artificial Sequence
  HhOM2-3′
ccgcatatgtcacggcgccgccgccagcaccgccaggtttgaatgcatgggagagcccag

SEQ ID NO: 199 - DNA, 45 bp, Artificial Sequence
  HhS-5′
ccgcatatgaagaagaagaggaagtacaacatgatggagacggag

SEQ ID NO: 200 - DNA, 36 bp, Artificial Sequence
  HhS-3′
ccgcatatgtcacatgtgtgagaggggcagtgtgcc

SEQ ID NO: 201 - DNA, 75 bp, Artificial Sequence
  HM1hS-5′
ccgcatatgaagaagaagaggaagctggtggcggcgctgctggcggtgctgtacaacatgatggagacggagctg

SEQ ID NO: 202 - DNA, 63 bp, Artificial Sequence
  HhSM1-3′
ccgcatatgtcacagcaccgccagcagcgccgccaccagcatgtgtgagaggggcagtgtgcc

SEQ ID NO: 203 - DNA, 72 bp, Artificial Sequence
  HM2hS-5′
ccgcatatgaagaagaagaggaagctggcggtgctggcggcggcgccgtacaacatgatggagacggagctg

SEQ ID NO: 204 - DNA, 60 bp, Artificial Sequence
  HhSM2-3′
ccgcatatgtcacggcgccgccgccagcaccgccagcatgtgtgagaggggcagtgtgcc

SEQ ID NO: 205 - DNA, 45 bp, Artificial Sequence
  HhK-5′
ccgcatatgaagaagaagaggaaggctgtcagcgacgcgctgctc

SEQ ID NO: 206 - DNA, 36 bp, Artificial Sequence
  HhK-3′
ccgcatatgttaaaaatgcctcttcatgtgtaaggc

SEQ ID NO: 207 - DNA, 75 bp, Artificial Sequence
  HM1hK-5′
ccgcatatgaagaagaagaggaagctggtggcggcgctgctggcggtgctggctgtcagcgacgcgctgctccca

SEQ ID NO: 208 - DNA, 63 bp, Artificial Sequence
  HhKM1-3′
ccgcatatgttacagcaccgccagcagcgccgccaccagaaaatgcctcttcatgtgtaaggc

SEQ ID NO: 209 - DNA, 72 bp, Artificial Sequence
  HM2hK-5′
ccgcatatgaagaagaagaggaagctggcggtgctggcggcggcgccggctgtcagcgacgcgctgctccca

SEQ ID NO: 210 - DNA, 60 bp, Artificial Sequence
  HhKM2-3′
ccgcatatgttacggcgccgccgccagcaccgccagaaaatgcctcttcatgtgtaaggc

SEQ ID NO: 211 - DNA, 45 bp, Artificial Sequence
  HhM-5′
ccgcatatgaagaagaagaggaaggatttttttcgggtagtggaa

SEQ ID NO: 212 - DNA, 36 bp, Artificial Sequence
  HhM-3′
ccgcatatgttacgcacaagagttccgtagctgttc

SEQ ID NO: 213 - DNA, 75 bp, Artificial Sequence
  HM1hM-5′
ccgcatatgaagaagaagaggaagctggtggcggcgctgctggcggtgctggatttttttcgggtagtggaaaac

SEQ ID NO: 214 - DNA, 63 bp, Artificial Sequence
  HhMM1-3′
ccgcatatgttacagcaccgccagcagcgccgccaccagcgcacaagagttccgtagctgttc

SEQ ID NO: 215 - DNA, 72 bp, Artificial Sequence
  HM2hM-5′
ccgcatatgaagaagaagaggaagctggcggtgctggcggcggcgccggatttttttcgggtagtggaaaac

SEQ ID NO: 216 - DNA, 60 bp, Artificial Sequence
  HhMM2-3′
ccgcatatgttacggcgccgccgccagcaccgccagcgcacaagagttccgtagctgttc

SEQ ID NO: 217 - DNA, 45 bp, Artificial Sequence
  HhL-5′
ccgggatccaagaagaagaggaagggctccgtgtccaaccagcag

SEQ ID NO: 218 - DNA, 36 bp, Artificial Sequence
  HhL-3′
ccgggatcctcaattctgagcctccgggagcagggt

SEQ ID NO: 219 - DNA, 75 bp, Artificial Sequence
  HM1hL-5′
ccgggatccaagaagaagaggaagctggtggcggcgctgctggcggtgctgggctccgtgtccaaccagcagttt

SEQ ID NO: 220 - DNA, 63 bp, Artificial Sequence
  HhLM1-3′
ccgggatccttacagcaccgccagcagcgccgccaccagtcaattctgagcctccgggagcag

SEQ ID NO: 221 - DNA, 72 bp, Artificial Sequence
  HM2hL-5′
ccgggatccaagaagaagaggaagctggcggtgctggcggcggcgccgggctccgtgtccaaccagcagttt

SEQ ID NO: 222 - DNA, 60 bp, Artificial Sequence
  HhLM2-3′
ccgggatccttacggcgccgccgccagcaccgccagtcaattctgagcctccgggagcag

SEQ ID NO: 223 - DNA, 48 bp, Artificial Sequence
  JO-10 MTD cDNA Sequence
ctgggcggcgcggtggtggcggcgccggtggcggcggcggtggcgccg

SEQ ID NO: 224 - protein, 16 aa, Artificial Sequence
  JO-10 MTD Amino Acid Sequence Sequence
LGGAVVAAPVAAAVAP

SEQ ID NO: 225 - DNA, 27 bp, Artificial Sequence
  JO-52 MTD cDNA Sequence
ccgctgctgctgctgctgccggcgctg

SEQ ID NO: 226 - protein, 9 aa, Artificial Sequence
  JO-52 MTD Amino Acid Sequence Sequence
PLLLLLPAL

SEQ ID NO: 227 - DNA, 36 bp, Artificial Sequence
  JO-132 MTD cDNA Sequence
gcggtggtggtgccggcgattgtgctggcggcgccg

SEQ ID NO: 228 - protein, 12 aa, Artificial Sequence
  JO-132 MTD Amino Acid Sequence Sequence
AVVVPAIVLAAP

SEQ ID NO: 229 - DNA, 30 bp, Artificial Sequence
  JO-145 MTD cDNA Sequence
gcggcggcgccggtgctgctgctgctgctg

SEQ ID NO: 230 - protein, 10 aa, Artificial Sequence
  JO-145 MTD Amino Acid Sequence Sequence
AAAPVLLLLL

SEQ ID NO: 231 - DNA, 27 bp, Artificial Sequence
  JO-173 MTD cDNA Sequence
gcggtgattccgattctggcggtgccg

SEQ ID NO: 232 - protein, 9 aa, Artificial Sequence
  JO-173 MTD Amino Acid Sequence Sequence
AVIPILAVP

SEQ ID NO: 233 - DNA, 27 bp, Artificial Sequence
  JO-181 MTD cDNA Sequence
gcggtgctgctgctgccggcggcggcg

SEQ ID NO: 234 - protein, 9 aa, Artificial Sequence
  JO-181 MTD Amino Acid Sequence Sequence
AVLLLPAAA

SEQ ID NO: 235 - DNA, 960 bp, Artificial Sequence
  Codon optimized His-NLS-Nanog cDNA Sequence
atgggttcaagtcatcatcatcatcatcataaaaaaaaacgcaaaagtgtggatccggcatgcccgcagtcactgccgtgttttgaagcgtcggactgcaaagaaagctctccgatgccggttatttgtggcccggaagaaaactatccgtcactgcagatgagttccgccgaaatgccgcataccgaaacggtcagcccgctgccgtcatcgatggatctgctgatccaagattcaccggacagctctacgtcgccgaaaggtaaacagccgaccagtgcagaaaactccgttgctaaaaaagaagataaagtgccggttaaaaaacaaaaaacccgtacggtctttagttccacgcagctgtgcgtgctgaatgaccgtttccagcgccaaaaatatctgagcctgcagcaaatgcaagaactgagcaacattctgaatctgtcttacaaacaggtgaaaacctggttccagaaccaacgtatgaaaagtaaacgctggcagaaaaacaattggccgaaaaactccaatggcgttacgcagaaagcgagtgccccgacctacccgtccctgtattcatcgtaccatcagggctgtctggtcaacccgaccggtaatctgccgatgtggtctaatcagacctggaacaatagtacgtggtccaaccagacccaaaatattcagagctggtctaaccacagctggaatacccagacgtggtgcacccaaagctggaacaatcaggcatggaactctccgttttataattgtggtgaagaatcactgcagtcgtgcatgcagttccaaccgaacagcccggcttctgatctggaagcggccctggaagcagctggcgaaggtctgaatgttatccagcaaaccacgcgctactttagcaccccgcaaacgatggacctgtttctgaattactcgatgaatatgcaaccggaagatgtgtaa

SEQ ID NO: 236 - DNA, 1008 bp, Artificial Sequence
  Codon optimized His-NLS-JO-10 MTD Nanog cDNA Sequence
atgggttcaagtcatcatcatcatcatcataaaaaaaaacgcaaactgggcggcgcggtcgtggcggctccggtggcagcagcggtggcaccgagtgtggatccggcatgcccgcagtcactgccgtgttttgaagcgtcggactgcaaagaaagctctccgatgccggttatttgtggcccggaagaaaactatccgtcactgcagatgagttccgccgaaatgccgcataccgaaacggtcagcccgctgccgtcatcgatggatctgctgatccaagattcaccggacagctctacgtcgccgaaaggtaaacagccgaccagtgcagaaaactccgttgctaaaaaagaagataaagtgccggttaaaaaacaaaaaacccgtacggtctttagttccacgcagctgtgcgtgctgaatgaccgtttccagcgccaaaaatatctgagcctgcagcaaatgcaagaactgagcaacattctgaatctgtcttacaaacaggtgaaaacctggttccagaaccaacgtatgaaaagtaaacgctggcagaaaaacaattggccgaaaaactccaatggcgttacgcagaaagcgagtgccccgacctacccgtccctgtattcatcgtaccatcagggctgtctggtcaacccgaccggtaatctgccgatgtggtctaatcagacctggaacaatagtacgtggtccaaccagacccaaaatattcagagctggtctaaccacagctggaatacccagacgtggtgcacccaaagctggaacaatcaggcatggaactctccgttttataattgtggtgaagaatcactgcagtcgtgcatgcagttccaaccgaacagcccggcttctgatctggaagcggccctggaagcagctggcgaaggtctgaatgttatccagcaaaccacgcgctactttagcaccccgcaaacgatggacctgtttctgaattactcgatgaatatgcaaccggaagatgtgtaa

SEQ ID NO: 237 - protein, 335 aa, Artificial Sequence
  Codon optimized His-NLS-JO-10 MTD Nanog Amino Acid Sequence
MGSSHHHHHHKKKRKLGGAVVAAPVAAAVAPSVDPACPQSLPCFEASDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 238 - DNA, 1125 bp, Artificial Sequence
  Codon optimized His-NLS-Oct4 cDNA Sequence
atgggttcgtcgcatcatcatcatcatcataaaaaaaaacgcaaagctggtcacctggcttccgattttgctttctcaccgccgccgggcggtggcggtgatggtccgggcggtccggaaccgggttgggtggacccgcgtacctggctgtccttccagggtccgccgggcggtccgggtattggtccgggtgttggtccgggctcagaagtctggggtatcccgccgtgcccgccgccgtatgaattttgcggtggtatggcatactgtggtccgcaggtcggtgtgggtctggttccgcagggcggtctggaaacgtcgcaaccggaaggcgaagcaggtgttggcgtcgaatcgaacagcgatggcgctagcccggaaccgtgtaccgtgacgccgggtgcggttaaactggaaaaagaaaaactggaacagaatccggaagaaagccaagatattaaagcgctgcagaaagaactggaacaattcgccaaactgctgaaacagaaacgcatcaccctgggttatacgcaagcggacgttggtctgaccctgggcgtcctgtttggtaaagtgttctcgcagaccacgatttgccgctttgaagctctgcaactgagcttcaaaaacatgtgtaaactgcgtccgctgctgcagaaatgggtcgaagaagcggataacaatgaaaatctgcaggaaatttgcaaagcagaaaccctggtgcaagctcgtaaacgcaaacgtacgtctatcgaaaaccgcgttcgtggcaacctggaaaatctgttcctgcagtgcccgaaaccgaccctgcagcaaattagccatatcgcccagcaactgggcctggaaaaagacgtggttcgtgtgtggttttgtaatcgtcgccagaaaggtaaacgcagctctagtgattacgcacagcgtgaagactttgaagcagcaggttctccgttcagtggcggtccggtctcttttccgctggcaccgggtccgcattttggtaccccgggttatggcagtccgcacttcacggcactgtactcctcagtgccgtttccggaaggcgaagcgttcccgccggtctccgtcaccaccctgggtagtccgatgcacagtaattaa

SEQ ID NO: 239 - DNA, 1152 bp, Artificial Sequence
  Codon optimized His-NLS-JO-52 MTD Oct4 cDNA Sequence
atgggttcgtcgcatcatcatcatcatcataaaaaaaaacgcaaaccgctgctgctgctgctgccggccctggctggtcacctggcttccgattttgctttctcaccgccgccgggcggtggcggtgatggtccgggcggtccggaaccgggttgggtggacccgcgtacctggctgtccttccagggtccgccgggcggtccgggtattggtccgggtgttggtccgggctcagaagtctggggtatcccgccgtgcccgccgccgtatgaattttgcggtggtatggcatactgtggtccgcaggtcggtgtgggtctggttccgcagggcggtctggaaacgtcgcaaccggaaggcgaagcaggtgttggcgtcgaatcgaacagcgatggcgctagcccggaaccgtgtaccgtgacgccgggtgcggttaaactggaaaaagaaaaactggaacagaatccggaagaaagccaagatattaaagcgctgcagaaagaactggaacaattcgccaaactgctgaaacagaaacgcatcaccctgggttatacgcaagcggacgttggtctgaccctgggcgtcctgtttggtaaagtgttctcgcagaccacgatttgccgctttgaagctctgcaactgagcttcaaaaacatgtgtaaactgcgtccgctgctgcagaaatgggtcgaagaagcggataacaatgaaaatctgcaggaaatttgcaaagcagaaaccctggtgcaagctcgtaaacgcaaacgtacgtctatcgaaaaccgcgttcgtggcaacctggaaaatctgttcctgcagtgcccgaaaccgaccctgcagcaaattagccatatcgcccagcaactgggcctggaaaaagacgtggttcgtgtgtggttttgtaatcgtcgccagaaaggtaaacgcagctctagtgattacgcacagcgtgaagactttgaagcagcaggttctccgttcagtggcggtccggtctcttttccgctggcaccgggtccgcattttggtaccccgggttatggcagtccgcacttcacggcactgtactcctcagtgccgtttccggaaggcgaagcgttcccgccggtctccgtcaccaccctgggtagtccgatgcacagtaattaa

SEQ ID NO: 240 - protein, 383 aa, Artificial Sequence
  Codon optimized His-NLS-JO-52 MTD Oct4 Amino Acid Sequence
MGSSHHHHHHKKKRKPLLLLLPALAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 241 - DNA, 996 bp, Artificial Sequence
  Codon optimized His-NLS-Sox2 cDNA Sequence
atgggctcgtcgcaccaccaccaccaccacaaaaaaaaacgcaaatataacatgatggaaaccgaactgaaaccgccgggtccgcagcaaacctcgggcggtggcggtggcaacagcacggcggcggcggcgggtggcaaccagaaaaattcaccggatcgtgtgaaacgcccgatgaatgcatttatggtttggagtcgtggtcagcgtcgcaaaatggctcaagaaaacccgaaaatgcataactctgaaatcagtaaacgtctgggcgcggaatggaaactgctgagcgaaaccgaaaaacgcccgttcatcgatgaagcgaaacgtctgcgcgccctgcatatgaaagaacacccggactataaataccgcccgcgtcgcaaaacgaaaacgctgatgaaaaaagataaatatacgctgccgggtggcctgctggcaccgggtggcaacagcatggcgtcaggtgttggtgtcggtgcaggtctgggtgcaggtgtcaatcagcgtatggattcttacgcccatatgaacggttggagtaatggcagttattccatgatgcaggaccaactgggttacccgcagcatccgggtctgaacgcacacggcgcagcacagatgcaaccgatgcaccgctatgacgtttccgccctgcagtacaactcaatgaccagctctcaaacgtatatgaatggctctccgacctattcaatgtcgtacagccagcagggtacgccgggtatggcactgggttcgatgggcagcgtggttaaaagcgaagccagttcctcaccgccggtcgtgacctcgagctctcactcccgtgcaccgtgccaggctggtgatctgcgcgacatgattagcatgtatctgccgggtgcagaagtgccggaaccggcagctccgtctcgtctgcatatgagtcagcactatcaatcgggtccggttccgggtacggctatcaacggtacgctgccgctgtcgcacatgtaa

SEQ ID NO: 242 - DNA, 1023 bp, Artificial Sequence
  Codon optimized His-NLS-JO-181 MTD Sox2 cDNA Sequence
atgggctcgtcgcaccaccaccaccaccacaaaaaaaaacgcaaagcggtcctgctgctgccggctgctgcctataacatgatggaaaccgaactgaaaccgccgggtccgcagcaaacctcgggcggtggcggtggcaacagcacggcggcggcggcgggtggcaaccagaaaaattcaccggatcgtgtgaaacgcccgatgaatgcatttatggtttggagtcgtggtcagcgtcgcaaaatggctcaagaaaacccgaaaatgcataactctgaaatcagtaaacgtctgggcgcggaatggaaactgctgagcgaaaccgaaaaacgcccgttcatcgatgaagcgaaacgtctgcgcgccctgcatatgaaagaacacccggactataaataccgcccgcgtcgcaaaacgaaaacgctgatgaaaaaagataaatatacgctgccgggtggcctgctggcaccgggtggcaacagcatggcgtcaggtgttggtgtcggtgcaggtctgggtgcaggtgtcaatcagcgtatggattcttacgcccatatgaacggttggagtaatggcagttattccatgatgcaggaccaactgggttacccgcagcatccgggtctgaacgcacacggcgcagcacagatgcaaccgatgcaccgctatgacgtttccgccctgcagtacaactcaatgaccagctctcaaacgtatatgaatggctctccgacctattcaatgtcgtacagccagcagggtacgccgggtatggcactgggttcgatgggcagcgtggttaaaagcgaagccagttcctcaccgccggtcgtgacctcgagctctcactcccgtgcaccgtgccaggctggtgatctgcgcgacatgattagcatgtatctgccgggtgcagaagtgccggaaccggcagctccgtctcgtctgcatatgagtcagcactatcaatcgggtccggttccgggtacggctatcaacggtacgctgccgctgtcgcacatgtaa

SEQ ID NO: 243 - protein, 340 aa, Artificial Sequence
  Codon optimized His-NLS-JO-181 MTD Sox2 Amino Acid Sequence
MGSSHHHHHHKKKRKAVLLLPAAAYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 244 - DNA, 1455 bp, Artificial Sequence
  Codon optimized His-NLS-Klf4 cDNA Sequence
atgggttcgagtcatcatcatcatcatcataaaaaaaaacgtaaagcagtgagcgacgcactgctgccgtcgtttagcacgttcgctagtggtccggcaggtcgtgaaaaaaccctgcgtcaggcaggtgcaccgaacaatcgttggcgcgaagaactgtcccacatgaaacgtctgccgccggtgctgccgggtcgtccgtatgatctggcggcggcaacggttgcaaccgacctggaatcaggcggtgctggtgctgcatgcggtggttcgaacctggcaccgctgccgcgtcgcgaaaccgaagaatttaacgatctgctggatctggacttcattctgtctaatagtctgacgcatccgccggaaagcgtcgcagcaaccgtgagctctagtgcttctgcgtcctcatcgagctctccgagttcctcaggtccggcatcggcaccgagcacgtgttcttttacctatccgatccgtgcaggcaatgatccgggtgttgctccgggcggtaccggcggtggcctgctgtacggtcgtgaaagcgctccgccgccgaccgcaccgtttaacctggccgatattaatgacgtttccccgtcaggtggcttcgtcgcagaactgctgcgtccggaactggacccggtctatatcccgccgcagcaaccgcaaccgccgggtggcggtctgatgggtaaatttgtgctgaaagcctcgctgagcgcaccgggcagcgaatatggttctccgagtgtgatttccgtttcaaaaggcagtccggatggttcccacccggtggttgtcgcaccgtacaacggtggtccgccgcgtacgtgcccgaaaatcaaacaagaagccgtgtcgagctgtacccatctgggtgcaggtccgccgctgtcaaatggtcaccgtccggctgcacatgatttcccgctgggtcgtcagctgccgtctcgtaccacgccgaccctgggtctggaagaagttctgtctagtcgtgactgccatccggcactgccgctgccgccgggctttcatccgcacccgggtccgaactatccgtctttcctgccggatcagatgcaaccgcaggtcccgccgctgcactaccaggaactgatgccgccgggcagttgtatgccggaagaaccgaaaccgaaacgtggtcgtcgctcctggccgcgtaaacgtaccgcaacgcatacctgcgactatgccggctgtggtaaaacgtacaccaaatcctcacatctgaaagcgcacctgcgcacgcataccggcgaaaaaccgtatcactgcgattgggacggctgtggttggaaatttgcccgtagcgatgaactgacgcgtcattaccgcaaacataccggtcaccgcccgttccaatgccagaaatgcgaccgcgccttctcccgttccgaccacctggcactgcacatgaaacgtcacttctaa

SEQ ID NO: 245 - DNA, 1482 bp, Artificial Sequence
  Codon optimized His-NLS-JO-173 MTD Klf4 cDNA Sequence
atgggttcgagtcatcatcatcatcatcataaaaaaaaacgtaaagcagtcatcccgattctggcggtcccggcagtgagcgacgcactgctgccgtcgtttagcacgttcgctagtggtccggcaggtcgtgaaaaaaccctgcgtcaggcaggtgcaccgaacaatcgttggcgcgaagaactgtcccacatgaaacgtctgccgccggtgctgccgggtcgtccgtatgatctggcggcggcaacggttgcaaccgacctggaatcaggcggtgctggtgctgcatgcggtggttcgaacctggcaccgctgccgcgtcgcgaaaccgaagaatttaacgatctgctggatctggacttcattctgtctaatagtctgacgcatccgccggaaagcgtcgcagcaaccgtgagctctagtgcttctgcgtcctcatcgagctctccgagttcctcaggtccggcatcggcaccgagcacgtgttcttttacctatccgatccgtgcaggcaatgatccgggtgttgctccgggcggtaccggcggtggcctgctgtacggtcgtgaaagcgctccgccgccgaccgcaccgtttaacctggccgatattaatgacgtttccccgtcaggtggcttcgtcgcagaactgctgcgtccggaactggacccggtctatatcccgccgcagcaaccgcaaccgccgggtggcggtctgatgggtaaatttgtgctgaaagcctcgctgagcgcaccgggcagcgaatatggttctccgagtgtgatttccgtttcaaaaggcagtccggatggttcccacccggtggttgtcgcaccgtacaacggtggtccgccgcgtacgtgcccgaaaatcaaacaagaagccgtgtcgagctgtacccatctgggtgcaggtccgccgctgtcaaatggtcaccgtccggctgcacatgatttcccgctgggtcgtcagctgccgtctcgtaccacgccgaccctgggtctggaagaagttctgtctagtcgtgactgccatccggcactgccgctgccgccgggctttcatccgcacccgggtccgaactatccgtctttcctgccggatcagatgcaaccgcaggtcccgccgctgcactaccaggaactgatgccgccgggcagttgtatgccggaagaaccgaaaccgaaacgtggtcgtcgctcctggccgcgtaaacgtaccgcaacgcatacctgcgactatgccggctgtggtaaaacgtacaccaaatcctcacatctgaaagcgcacctgcgcacgcataccggcgaaaaaccgtatcactgcgattgggacggctgtggttggaaatttgcccgtagcgatgaactgacgcgtcattaccgcaaacataccggtcaccgcccgttccaatgccagaaatgcgaccgcgccttctcccgttccgaccacctggcactgcacatgaaacgtcacttctaa

SEQ ID NO: 246 - protein, 493 aa, Artificial Sequence
  Codon optimized His-NLS-JO-173 MTD Klf4 Amino Acid Sequence
MGSSHHHHHHKKKRKAVIPILAVPAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 247 - DNA, 1410 bp, Artificial Sequence
  Codon optimized His-NLS-cMyc cDNA Sequence
atgggttcgtcgcaccaccaccaccatcataaaaaaaaacgcaaactggacttcttccgtgtggttgaaaaccagcaaccgccggcgaccatgccgctgaatgtgtcctttacgaaccgcaattatgatctggactacgattcagttcaaccgtatttttactgcgacgaagaagaaaacttctatcagcaacagcaacagtccgaactgcagccgccggcaccgtcagaagatatctggaaaaaattcgaactgctgccgaccccgccgctgtcgccgagccgtcgctccggtctgtgttctccgagttacgtcgccgtgacgccgtttagtctgcgtggtgacaatgatggcggtggcggttccttctcaaccgcagatcaactggaaatggttacggaactgctgggcggtgacatggtcaaccagagctttatctgcgatccggatgacgaaaccttcatcaaaaacatcatcattcaggactgtatgtggtcgggctttagcgcggcggcaaaactggtctctgaaaaactggcaagttatcaggctgcgcgtaaagattcgggtagcccgaacccggcacgtggtcattctgtttgcagtaccagctctctgtatctgcaggacctgtcggccgcagctagcgaatgtatcgatccgagcgtcgtgtttccgtacccgctgaatgatagttcctcaccgaaatcctgcgcatcacaagactcgagcgctttctccccgtctagtgattcactgctgtcctcaaccgaatcgagcccgcagggttctccggaaccgctggtgctgcacgaagaaacgccgccgaccacgtctagtgattctgaagaagaacaagaagacgaagaagaaattgatgttgtcagcgttgaaaaacgtcaggcgccgggcaaacgctctgaaagtggttccccgtcagccggcggtcattcgaaaccgccgcacagcccgctggtgctgaaacgttgccatgtttctacccatcagcacaactatgcggccccgccgagtacgcgtaaagactacccggcagctaaacgcgtgaaactggatagcgttcgtgtcctgcgccagatttctaacaatcgtaaatgtaccagtccgcgctcctcagatacggaagaaaacgtcaaacgtcgcacccacaatgtgctggaacgccaacgtcgcaatgaactgaaacgtagctttttcgcactgcgcgatcagatcccggaactggaaaacaatgaaaaagctccgaaagtggttatcctgaaaaaagcgaccgcctatattctgtcggtgcaagcggaagaacagaaactgattagcgaagaagatctgctgcgtaaacgtcgtgaacaactgaaacacaaactggaacaactgcgtaattcttgcgcttaa

SEQ ID NO: 248 - DNA, 1440 bp, Artificial Sequence
  Codon optimized His-NLS-JO-145 MTD cMyc cDNA Sequence
atgggttcgtcgcaccaccaccaccatcataaaaaaaaacgcaaagcggctgccccggttctgctgctgctgctgctggacttcttccgtgtggttgaaaaccagcaaccgccggcgaccatgccgctgaatgtgtcctttacgaaccgcaattatgatctggactacgattcagttcaaccgtatttttactgcgacgaagaagaaaacttctatcagcaacagcaacagtccgaactgcagccgccggcaccgtcagaagatatctggaaaaaattcgaactgctgccgaccccgccgctgtcgccgagccgtcgctccggtctgtgttctccgagttacgtcgccgtgacgccgtttagtctgcgtggtgacaatgatggcggtggcggttccttctcaaccgcagatcaactggaaatggttacggaactgctgggcggtgacatggtcaaccagagctttatctgcgatccggatgacgaaaccttcatcaaaaacatcatcattcaggactgtatgtggtcgggctttagcgcggcggcaaaactggtctctgaaaaactggcaagttatcaggctgcgcgtaaagattcgggtagcccgaacccggcacgtggtcattctgtttgcagtaccagctctctgtatctgcaggacctgtcggccgcagctagcgaatgtatcgatccgagcgtcgtgtttccgtacccgctgaatgatagttcctcaccgaaatcctgcgcatcacaagactcgagcgctttctccccgtctagtgattcactgctgtcctcaaccgaatcgagcccgcagggttctccggaaccgctggtgctgcacgaagaaacgccgccgaccacgtctagtgattctgaagaagaacaagaagacgaagaagaaattgatgttgtcagcgttgaaaaacgtcaggcgccgggcaaacgctctgaaagtggttccccgtcagccggcggtcattcgaaaccgccgcacagcccgctggtgctgaaacgttgccatgtttctacccatcagcacaactatgcggccccgccgagtacgcgtaaagactacccggcagctaaacgcgtgaaactggatagcgttcgtgtcctgcgccagatttctaacaatcgtaaatgtaccagtccgcgctcctcagatacggaagaaaacgtcaaacgtcgcacccacaatgtgctggaacgccaacgtcgcaatgaactgaaacgtagctttttcgcactgcgcgatcagatcccggaactggaaaacaatgaaaaagctccgaaagtggttatcctgaaaaaagcgaccgcctatattctgtcggtgcaagcggaagaacagaaactgattagcgaagaagatctgctgcgtaaacgtcgtgaacaactgaaacacaaactggaacaactgcgtaattcttgcgcttaa

SEQ ID NO: 249 - protein, 479 aa, Artificial Sequence
  Codon optimized His-NLS-JO-145 MTD cMyc Amino Acid Sequence
MGSSHHHHHHKKKRKAAAPVLLLLLLDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 250 - DNA, 672 bp, Artificial Sequence
  Codon optimized His-NLS-Lin28 cDNA Sequence
atgggctcgtcgcatcatcatcatcatcataaaaaaaaacgcaaaggttcggttagcaaccagcaatttgcgggcggttgcgccaaagcggccgaagaagcaccggaagaagctccggaagatgcagctcgtgcagcagacgaaccgcagctgctgcatggcgcaggtatttgtaaatggttcaatgtccgtatgggctttggtttcctgtctatgaccgcacgtgctggtgtggcactggatccgccggtggacgtttttgtccatcaaagcaaactgcacatggaaggcttccgttctctgaaagaaggtgaagctgttgaatttaccttcaaaaaatctgccaaaggcctggaatccattcgcgtgacgggtccgggcggtgtgttttgcatcggcagcgaacgtcgcccgaagggtaaatcaatgcagaaacgtcgctcgaaaggtgatcgctgctataactgtggcggtctggaccatcacgccaaagaatgcaaactgccgccgcagccgaaaaaatgccatttctgtcaaagcatctctcacatggtcgcgagttgtccgctgaaagcccagcaaggcccgtccgcacagggtaaaccgacgtactttcgtgaagaagaagaagaaatccactccccgacgctgctgccggaagcccagaactaa

SEQ ID NO: 251 - DNA, 708 bp, Artificial Sequence
  Codon optimized His-NLS-JO-132 MTD Lin28 cDNA Sequence
atgggctcgtcgcatcatcatcatcatcataaaaaaaaacgcaaagcagtcgtcgtcccggcaatcgtcctggcggcaccgggttcggttagcaaccagcaatttgcgggcggttgcgccaaagcggccgaagaagcaccggaagaagctccggaagatgcagctcgtgcagcagacgaaccgcagctgctgcatggcgcaggtatttgtaaatggttcaatgtccgtatgggctttggtttcctgtctatgaccgcacgtgctggtgtggcactggatccgccggtggacgtttttgtccatcaaagcaaactgcacatggaaggcttccgttctctgaaagaaggtgaagctgttgaatttaccttcaaaaaatctgccaaaggcctggaatccattcgcgtgacgggtccgggcggtgtgttttgcatcggcagcgaacgtcgcccgaagggtaaatcaatgcagaaacgtcgctcgaaaggtgatcgctgctataactgtggcggtctggaccatcacgccaaagaatgcaaactgccgccgcagccgaaaaaatgccatttctgtcaaagcatctctcacatggtcgcgagttgtccgctgaaagcccagcaaggcccgtccgcacagggtaaaccgacgtactttcgtgaagaagaagaagaaatccactccccgacgctgctgccggaagcccagaactaa

SEQ ID NO: 252 - protein, 235 aa, Artificial Sequence
  Codon optimized His-NLS-JO-132 MTD Lin28 Amino Acid Sequence
MGSSHHHHHHKKKRKAVVVPAIVLAAPGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN